(19) United States
(12) Patent Application Publication
(10) Pub. No.: US 2024/0009203 A1
(43) Pub. Date: Jan. 11, 2024

(54) COMPOSITIONS AND METHODS FOR TREATING ALZHEIMER'S DISEASE

(71) Applicants: Cedars-Sinai Medical Center, Los Angeles, CA (US); Fortem Neurosciences, Inc., Sunny Isles Beach, FL (US)

(72) Inventors: Maya KORONYO, Los Angeles, CA (US); Jack KAVANAUGH, Los Angeles, CA (US); Keith L. BLACK, Los Angeles, CA (US)

(21) Appl. No.: 18/040,310

(22) PCT Filed: Aug. 2, 2021
(86) PCT No.: PCT/US2021/044195

§ 371 Date: Feb. 2, 2023

Related U.S. Application Data
(60) Provisional application No. 63/060,553, filed on Aug. 3, 2020.

(51) Int. Cl. A61K 31/548; A61P 25/28

(52) U.S. Cl. CPC A61K 31/548; A61P 25/28

(57) ABSTRACT

Provided herein are compositions and methods that improve cognition in individuals having or at risk of having Alzheimers disease, including familial Alzheimers disease, early-onset Alzheimers disease, and middle to late stage Alzheimers disease.

### CROSS-REFERENCE

This application claims the benefit of U.S. Provisional Application No. 63/060,553, filed Aug. 3, 2020, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 21, 2021, is named “50014_708_601_sequence_listing.txt” and is 14,845 bytes in size.

### BACKGROUND

There is an urgent unmet need to develop effective Alzheimer's disease treatments, and for preserving cognitive function in subjects with Alzheimer's disease or at risk of developing Alzheimer's disease.

### SUMMARY

Disclosed herein, in some embodiments, are methods of treating, preventing or delaying onset of an Alzheimer's disease in a subject in need thereof, the method comprising administering to the subject a composition comprising ETP69 wherein the subject has (i) at least one mutation associated with familial Alzheimer's disease; or (ii) at least one genetic risk factor associated with sporadic Alzheimer's disease. In some embodiments, the familial Alzheimer's disease comprises Alzheimer's disease type 1. In some embodiments, the familial Alzheimer's disease comprises Alzheimer's disease type 3. In some embodiments, the familial Alzheimer's disease comprises Alzheimer's disease type 4. In some embodiments, the at least one mutation associated with familial Alzheimer's disease comprises a mutation in an amyloid precursor protein (APP) gene, a presenilin-1 (PSEN1) gene, or a presenilin-2 (PSEN2) gene. In some embodiments, the mutation in the APP gene codes for a mutation in the amyloid precursor protein amino acid sequence that is expressed from the APP gene. In some embodiments, the mutation in the amyloid precursor protein amino acid sequence comprises a mutation at valine 717 according to SEQ ID NO: 1. In some embodiments, the valine 717 of the amyloid precursor protein is mutated to isoleucine (V717I), phenylalanine (V717F), glycine (V717G), or leucine (V717L). In some embodiments, the mutation in the amyloid precursor protein amino acid sequence comprises mutations at positions lysine 670 and methionine 671 according to SEQ ID NO: 1. In some embodiments, the lysine 670 and methionine 671 are mutated to asparagine and lysine, respectively (KM670/671NL). In some embodiments, the mutation in the amyloid precursor protein amino acid sequence comprises a mutation at glutamate 693 according to SEQ ID NO: 1. In some embodiments, the mutation in the APP gene codes for a deletion at position glutamate 693 of the amyloid precursor protein amino acid. In some embodiments, the glutamate 693 of the of the amyloid precursor protein is mutated to glutamine (E693Q), glycine (E693G), or lysine (E693K). In some embodiments, the mutation in the amyloid precursor protein amino acid sequence comprises a mutation at threonine 714 according to SEQ ID NO: 1. In some embodiments, the threonine 714 of the of the amyloid precursor protein is mutated to isoleucine (T714I) or alanine (T714A). In some embodiments, the mutation in the amyloid precursor protein amino acid sequence comprises a mutation at valine 715 according to SEQ ID NO: 1. In some embodiments, the valine 715 of the of the amyloid precursor protein is mutated to methionine (V715M) or alanine (V715A). In some embodiments, the mutation in the amyloid precursor protein amino acid sequence comprises a mutation at isoleucine 716 according to SEQ ID NO: 1. In some embodiments, the isoleucine 716 of the of the amyloid precursor protein is mutated to valine (I716V) or phenylalanine (I716F). In some embodiments, the mutation in the PSEN-1 gene codes for a mutation in the presenilin-1 amino acid sequence that is expressed from the PSEN-1 gene. In some embodiments, the mutation in the presenilin-1 amino acid sequence comprises a mutation at methionine 146, leucine 166, isoleucine 213, arginine 278, or alanine 246 according to SEQ ID NO: 2. In some embodiments, the methionine 146 is mutated to leucine (M146L), the leucine 166 is mutated to proline (L166P), the isoleucine 213 is mutated to threonine (I213T), the arginine 278 is mutated to isoleucine (R278I), and the alanine 246 is mutated to glutamate (A246E). In some embodiments, the mutation in the PSEN-2 gene codes for a mutation in the presenilin-2 amino acid sequence that is expressed from the PSEN-2 gene. In some embodiments, the mutation in the presenilin-2 amino acid sequence comprises a mutation at asparagine 141 or methionine 239 according to SEQ ID NO: 3. In some embodiments, the asparagine 141 is mutated to isoleucine and the methionine 239 is mutated to valine. In some embodiments, the genetic risk factor associated with sporadic Alzheimer's disease comprises the subject being a carrier of an apolipoprotein (APOE) e4 allele. In some embodiments, the genetic risk factor associated with sporadic Alzheimer's disease comprises a mutation in a gene. In some embodiments, the gene comprises ABCA7, AKAP9, BIN1, CASS4, CD2AP, CD33, CLU, EPHA1, FERMT2, HLA-DRB5/DRB1, INPP5D, MEF2C, MS4A6A/MS4A4E, PICALM, PLD3, ACE, PTK2B, —SORL1, TREM2, or UNC5C. In some embodiments, the subject is asymptomatic of Alzheimer's disease. In some embodiments, the subject is less than about 25, about 30, about 35, about 40, about 45, about 50, about, about 55, about 60, about 65, or about 70 years of age. In some embodiments, the delaying onset of Alzheimer's disease comprises a delay in onset of at least one symptom of Alzheimer's disease. In some embodiments, the delay in onset of at least on symptom is at least about 6 months, about 12 months, about 18 months, about 2 years, about 3 years, about 5 years, about 10 years, about 15 years, or about 20 years. In some embodiments, the symptom comprises memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes. In some embodiments, the method improves memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes in the subject. In some embodiments, the treatment results in improvement in a mental status test and/or a neuroimaging test. In some embodiments, the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test. In some embodiments, the improvement comprises an improved score relative to a score obtained prior to administration of the composition. In some embodiments, the neuroimaging test is a magnetic resonance imaging (MRI) or computed tomography (CT). In some embodiments, the improvement comprises reduced beta-amyloid deposits as compared to an amount of beta-amyloid deposits measured prior to administration of the composition.

Disclosed herein, in some embodiments, are methods of improving cognition in a subject at risk of developing Alzheimer's disease, the method comprising administering to the subject a composition comprising ETP69 wherein the subject has (i) at least one mutation associated with familial Alzheimer's disease; or (ii) at least one genetic risk factor associated with sporadic Alzheimer's disease. In some embodiments, the familial Alzheimer's disease comprises Alzheimer's disease type 1. In some embodiments, the familial Alzheimer's disease comprises Alzheimer's disease type 3. In some embodiments, the familial Alzheimer's disease comprises Alzheimer's disease type 4. In some embodiments, the at least one mutation associated with familial Alzheimer's disease comprises a mutation in an amyloid precursor protein (APP) gene, a presenilin-1 (PSEN1) gene, or a presenilin-2 (PSEN2) gene. In some embodiments, the mutation in the APP gene codes for a mutation in the amyloid precursor protein amino acid sequence that is expressed from the APP gene. In some embodiments, the mutation in the amyloid precursor protein amino acid sequence comprises a mutation at valine 717 according to SEQ ID NO: 1. In some embodiments, the valine 717 of the amyloid precursor protein is mutated to isoleucine (V717I), phenylalanine (V717F), glycine (V717G), or leucine (V717L). In some embodiments, the mutation in the amyloid precursor protein amino acid sequence comprises mutations at positions lysine 670 and methionine 671 according to SEQ ID NO: 1. In some embodiments, the lysine 670 and methionine 671 are mutated to asparagine and lysine, respectively (KM670/671NL). In some embodiments, the mutation in the amyloid precursor protein amino acid sequence comprises a mutation at glutamate 693 according to SEQ ID NO: 1. In some embodiments, the mutation in the APP gene codes for a deletion at position glutamate 693 of the amyloid precursor protein amino acid. In some embodiments, the glutamate 693 of the of the amyloid precursor protein is mutated to glutamine (E693Q), glycine (E693G), or lysine (E693K). In some embodiments, the mutation in the amyloid precursor protein amino acid sequence comprises a mutation at threonine 714 according to SEQ ID NO: 1. In some embodiments, the threonine 714 of the of the amyloid precursor protein is mutated to isoleucine (T714I) or alanine (T714A). In some embodiments, the mutation in the amyloid precursor protein amino acid sequence comprises a mutation at valine 715 according to SEQ ID NO: 1. In some embodiments, the valine 715 of the of the amyloid precursor protein is mutated to methionine (V715M) or alanine (V715A). In some embodiments, the mutation in the amyloid precursor protein amino acid sequence comprises a mutation at isoleucine 716 according to SEQ ID NO: 1. In some embodiments, the isoleucine 716 of the of the amyloid precursor protein is mutated to valine (I716V) or phenylalanine (I716F). In some embodiments, the mutation in the PSEN-1 gene codes for a mutation in the presenilin-1 amino acid sequence that is expressed from the PSEN-1 gene. In some embodiments, the mutation in the presenilin-1 amino acid sequence comprises a mutation at methionine 146, leucine 166, isoleucine 213, arginine 278, or alanine 246 according to SEQ ID NO: 2. In some embodiments, the methionine 146 is mutated to leucine (M146L), the leucine 166 is mutated to proline (L166P), the isoleucine 213 is mutated to threonine (I213T), the arginine 278 is mutated to isoleucine (R278I), and the alanine 246 is mutated to glutamate (A246E). In some embodiments, the mutation in the PSEN-2 gene codes for a mutation in the presenilin-2 amino acid sequence that is expressed from the PSEN-2 gene. In some embodiments, the mutation in the presenilin-2 amino acid sequence comprises a mutation at asparagine 141 or methionine 239 according to SEQ ID NO: 3. In some embodiments, the asparagine 141 is mutated to isoleucine and the methionine 239 is mutated to valine. In some embodiments, the genetic risk factor associated with sporadic Alzheimer's disease comprises the subject being a carrier of apolipoprotein (APOE) e4 allele. In some embodiments, the genetic risk factor associated with sporadic Alzheimer's disease comprises a mutation in a gene. In some embodiments, the gene comprises ABCA7, AKAP9, BIN1, CASS4, CD2AP, CD33, CLU, EPHA1, FERMT2, HLA-DRB5/DRB1, INPP5D, MEF2C, MS4A6A/MS4A4E, PICALM, PLD3, ACE, PTK2B, —SORL1, TREM2, or UNC5C. In some embodiments, the subject is asymptomatic of Alzheimer's disease. In some embodiments, the subject is less than about 25, about 30, about 35, about 40, about 45, about 50, about, about 55, about 60, about 65, or about 70 years of age. In some embodiments, the method improves memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes in the subject. In some embodiments, the treatment results in improvement in a mental status test and/or a neuroimaging test. In some embodiments, the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test. In some embodiments, the improvement comprises an improved score relative to a score obtained prior to administration of the composition. In some embodiments, the neuroimaging test is a magnetic resonance imaging (MRI) or computed tomography (CT). In some embodiments, the improvement comprises reduced beta-amyloid deposits as compared to an amount of beta-amyloid deposits measured prior to administration of the composition.

Disclosed herein, in some embodiments, are methods of improving cognition in a subject in need thereof, the method comprising: (a) obtaining results of a genetic test for at least one mutation associated with familial Alzheimer's disease for the subject; and (b) administering a composition comprising ETP69 to the subject when the genetic test indicates that the subject has at least one mutation associated with familial Alzheimer's disease. In some embodiments, the familial Alzheimer's disease comprises Alzheimer's disease type 1. In some embodiments, the familial Alzheimer's disease comprises Alzheimer's disease type 3. In some embodiments, the familial Alzheimer's disease comprises Alzheimer's disease type 4. In some embodiments, the at least one mutation associated with familial Alzheimer's disease comprises a mutation in an amyloid precursor protein (APP) gene, a presenilin-1 (PSEN1) gene, or a presenilin-2 (PSEN2) gene. In some embodiments, the mutation in the APP gene codes for a mutation in the amyloid precursor protein amino acid sequence that is expressed from the APP gene. In some embodiments, the mutation in the amyloid precursor protein amino acid sequence comprises a mutation at valine 717 according to SEQ ID NO: 1. In some embodiments, the valine 717 of the amyloid precursor protein is mutated to isoleucine (V717I), phenylalanine (V717F), glycine (V717G), or leucine (V717L). In some embodiments, the mutation in the amyloid precursor protein amino acid sequence comprises mutations at positions lysine 670 and methionine 671 according to SEQ ID NO: 1. In some embodiments, the lysine 670 and methionine 671 are mutated to asparagine and lysine, respectively (KM670/671NL). In some embodiments, the mutation in the amyloid precursor protein amino acid sequence comprises a mutation at glutamate 693 according to SEQ ID NO: 1. In some embodiments, the mutation in the APP gene codes for a deletion at position glutamate 693 of the amyloid precursor protein amino acid. In some embodiments, the glutamate 693 of the of the amyloid precursor protein is mutated to glutamine (E693Q), glycine (E693G), or lysine (E693K). In some embodiments, the mutation in the amyloid precursor protein amino acid sequence comprises a mutation at threonine 714 according to SEQ ID NO: 1. In some embodiments, the threonine 714 of the of the amyloid precursor protein is mutated to isoleucine (T714I) or alanine (T714A). In some embodiments, the mutation in the amyloid precursor protein amino acid sequence comprises a mutation at valine 715 according to SEQ ID NO: 1. In some embodiments, the valine 715 of the of the amyloid precursor protein is mutated to methionine (V715M) or alanine (V715A). In some embodiments, the mutation in the amyloid precursor protein amino acid sequence comprises a mutation at isoleucine 716 according to SEQ ID NO: 1. In some embodiments, the isoleucine 716 of the of the amyloid precursor protein is mutated to valine (I716V) or phenylalanine (I716F). In some embodiments, the mutation in the PSEN-1 gene codes for a mutation in the presenilin-1 amino acid sequence that is expressed from the PSEN-1 gene. In some embodiments, the mutation in the presenilin-1 amino acid sequence comprises a mutation at methionine 146, leucine 166, isoleucine 213, arginine 278, or alanine 246 according to SEQ ID NO: 2. In some embodiments, the methionine 146 is mutated to leucine (M146L), the leucine 166 is mutated to proline (L166P), the isoleucine 213 is mutated to threonine (I213T), the arginine 278 is mutated to isoleucine (R278I), and the alanine 246 is mutated to glutamate (A246E). In some embodiments, the mutation in the PSEN-2 gene codes for a mutation in the presenilin-2 amino acid sequence that is expressed from the PSEN-2 gene. In some embodiments, the mutation in the presenilin-2 amino acid sequence comprises a mutation at asparagine 141 or methionine 239 according to SEQ ID NO: 3. In some embodiments, the asparagine 141 is mutated to isoleucine and the methionine 239 is mutated to valine.

Disclosed herein, in some embodiments, are methods of improving cognition in a subject in need thereof, the method comprising: (a) obtaining results of a genetic test for at least one mutation associated with sporadic Alzheimer's disease for the subject; and (b) administering a composition comprising ETP69 to the subject when the genetic test indicates that the subject has at least one mutation associated with sporadic Alzheimer's disease. In some embodiments, the at least one mutation associated with sporadic Alzheimer's disease comprises a mutation in an apolipoprotein (APOE) e4 gene. In some embodiments, the mutation in the APOE e4 gene codes for a mutation in the apolipoprotein e4 amino acid sequence that is expressed from the APOE e4 gene. In some embodiments, the genetic risk factor associated with sporadic Alzheimer's disease comprises a mutation in a gene. In some embodiments, the gene comprises ABCA7, AKAP9, BIN1, CASS4, CD2AP, CD33, CLU, EPHA1, FERMT2, HLA-DRB5/DRB1, INPP5D, MEF2C, MS4A6A/MS4A4E, PICALM, PLD3, ACE, PTK2B, —SORL1, TREM2, or UNC5C.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 is a chemical structure of a compound described herein.

FIG. 2A is an experimental design that includes 3 cohorts.

FIG. 2B is an experimental design for a first cohort.

FIG. 2C is an experimental design for a second cohort.

FIG. 2D is an experimental design for a third cohort.

FIG. 3A is a graph of total entries in a Y-maze experiment.

FIG. 3B is a graph of percent spontaneous alteration in a Y-maze experiment.

FIG. 4A is a graph of total entries in a visual X-maze (color, E) experiment.

FIG. 4B is a graph of percent spontaneous alteration in a visual X-maze (color, E) experiment.

FIG. 4C includes pictorial descriptions of a visual X-maze (color, E) experiment that was used.

FIG. 4D is a graph of total entries in a visual X-maze (contrast) experiment.

FIG. 4E is a graph of percent spontaneous alteration in a visual X-maze (contrast) experiment.

FIG. 4F includes pictorial descriptions of a visual X-maze (contrast) experiment that was used.

FIG. 4G is a graph of percent blue entries in a visual X-maze (color, entries) experiment.

FIG. 4H includes a pictorial description of a visual X-maze (color, entries) experiment that was used.

FIG. 4I is a graph of percent blue-white in a visual X-maze (color, bidirectional) experiment, as well as a pictorial description of the visual X-maze (color, bidirectional) experiment that was used.

FIG. 4J is a graph of percent red-white in a visual X-maze (color, bidirectional) experiment, as well as a pictorial description of the visual X-maze (color, bidirectional) experiment that was used.

FIG. 5A includes graphs of amounts of rearing, total activity, and average speed in an open field test.

FIG. 5B includes a plot of incorrect entries in a Barnes maze, and graphs of incorrect entries and latency.

FIG. 5C includes pictorial descriptions of a Barnes maze test that was used.

FIG. 6A is a plot of freezing times in a fear conditioning test.

FIG. 6B includes graphs of freezing times in a fear conditioning test.

FIG. 7A includes images of Golgi-Cox stained neurons and dendritic projections in wild-type mice at 15 months.

FIG. 7B includes images of Golgi-Cox stained neurons and dendritic projections in K1 wild-type mice at 18 months.

FIG. 7C includes images of Golgi-Cox stained neurons and dendritic projections in K5 ADtg mice and K1 wild-type mice treated with ETP69 at 18 months.

FIG. 7D includes images of Golgi-Cox stained neurons and dendritic projections in K5 ADtg mice treated with ETP69 at 18 months.

FIG. 8 depicts the experimental protocol used to test the effects of ETP69 on 18 month mice.

FIG. 9A depicts numbers of total entries into the Y maze.

FIG. 9B depicts percent of alternations in the Y maze.

FIG. 10A depicts a schematic of the visual stimuli X maze and the number of total entries into the visual stimuli X maze.

FIG. 10B depicts percent of alternations in the visual stimuli X maze.

FIG. 10C depicts percent transitions in the visual stimuli X maze.

FIG. 10D depicts percent of alternations in the visual stimuli X maze.

FIG. 10E depicts a schematic of the high-contrast visual stimuli X maze as well as the total number of entries.

FIG. 10F depicts percent of alternations in the high contrast visual stimuli X maze.

FIG. 11A depicts a schematic of the Barnes maze test and the number of errors over during training

FIG. 11B depicts numbers of errors in the retention phase of the Barnes maze test.

FIG. 11C depicts numbers of errors that occur in the reversal phase of the Barnes maze test.

FIG. 11D depicts the number of errors during training in mice that received a single injection.

FIG. 11E depicts the number of errors in training of mice that received repeated injections.

FIG. 11F depicts a comparison of the number of errors in mice that received single, boost, or repeated injections.

FIG. 11G depicts the number of errors in the reversal phase of mice that received a single injection.

FIG. 11H depicts the number of errors in the reversal phase of mice that received repeated injections.

FIG. 12 depicts the freezing time that occurred in the contextual fear conditioning test.

FIG. 13A depicts representative sections of Golgi-Cox staining in the Cingulate Cortex (CC) of mice treated with ETP69 and DMSO.

FIG. 13B depicts representative sections of Golgi-Cox staining in the Hippocampal area (Hipp) of mice treated with ETP69.

FIG. 13C depicts a quantification of the number of dendritic spines and thin spines of in the Cingulate Cortex of mice treated with ETP69 and DMSO.

FIG. 13D depicts a quantification of the number of dendritic spines and thin spines of in the Hippocampal area of mice treated with ETP69 and DMSO.

FIG. 13E depicts a quantification of the ratio of thin spines to all spines in the Cingulate Cortex (CC) and Hippocampal area (Hipp) of mice treated with ETP69 and DMSO.

FIG. 13F depicts a quantification of the ratio of thin spines to all spines in the Cingulate Cortex (CC) and Hippocampal area (Hipp) of mice treated with ETP69 and DMSO.

FIG. 13G depicts a correlation of the ratio of thin spines to total spines with the number of errors in the retention phase of the Barnes maze test.

FIG. 14A depicts H3K9me3 signal across the cortical layers.

FIG. 14B depicts H3K9me3 and DAPI staining in AD+ mice treated with ETP69 and DMSO.

FIG. 14C depicts a quantification of H3Kme3 and the ratio of H3K9me3 to actin in the Cingulate Cortex (CC) and Hippocampal area (Hipp) of mice treated with ETP69 and DMSO.

FIG. 14D depicts a representative image of 6E10 and GFAP staining in AD+ mice treated with ETP69 and DMSO.

FIG. 14E depicts a quantification of 6E10 in the Cingulate Cortex (CC) of mice treated with ETP69 and DMSO.

FIG. 14F depicts a quantification of GFAP in the Cingulate Cortex (CC) of mice treated with ETP69 and DMSO.

FIG. 14G a quantification of the ratio of GFAP to actin in the Hippocampal area (Hipp) of mice treated with ETP69 and DMSO.

FIG. 15 depicts the experimental protocol for testing the effect of ETP69 in 14 month mice.

FIG. 16A depicts a schematic of the visual stimuli X maze and the number of total entries into the visual stimuli X maze.

FIG. 16B depicts percent of alternations in the visual stimuli X maze.

FIG. 16C depicts a pictorial description of a visual X-maze experiment that was used.

FIG. 16D depicts percent transitions in the visual stimuli X maze.

FIG. 17A depicts results of the fear condition test in wildtype and AD+ mice treated with ETP69 and DMSO.

FIG. 17B depicts effects of ETP69 treatment on freezing time in the fear conditioning test.

FIG. 18A depicts representative images of H3K9me2, 6E10, and GFAP staining in mice administered DMSO and ETP69.

FIG. 18B depicts a quantification of H3K9me3 staining in mice administered DMSO and ETP69.

FIG. 18C depicts a quantification of 6E10 staining in mice administered DMSO and ETP69.

FIG. 18D depicts a quantification of GFAP staining in mice administered DMSO and ETP69.

FIG. 18E depicts a quantification of Iba1 staining in mice administered DMSO and ETP69.

FIG. 19A depicts a proteomics analysis comparing brains of mice treated with ETP and untreated mice. The figure includes a volcano plot of top downregulated proteins (left) and top upregulated proteins (right).

FIG. 19B depicts an ingenuity pathway analysis showing BDNF pathway activation.

FIG. 20 depicts behavioral pathway results of the ingenuity software analysis.

FIG. 21A depicts effects of ETP69 treatment on H3K9me3 staining in myelomonocytic cells.

FIG. 21B depicts effects of ETP69 treatment on 6E10 staining in myelomonocytic cells.

FIG. 21C depicts effects of ETP69 treatment on GFAP staining in myelomonocytic cells.

FIG. 21D depicts a representative image of Iba-1 staining in AD+ mice treated with DMSO or ETP69.

FIG. 21E depicts a quantification of Iba-1 staining in AD+ mice treated with DMSO or ETP69.

FIG. 21F depicts a comparison between the percent alternation and the H3K9me3 staining.

FIG. 21G depicts a representative staining of H3K9me3, —NeuN, and DAPI in AD+ mice administered DMSO or ETP69.

FIG. 21H depicts a representative staining of H3K9me3, CD45, Iba-1, 6E10 and DAPI staining in AD+ mice administered DMSO or ETP69.

FIG. 21I depicts a quantification of H3K9me3 staining in neurons of mice administered ETP69 or DMSO.

FIG. 21J depicts a quantification of H3K9me3 staining in microglia of mice administered ETP69 or DMSO.

FIG. 21K depicts a representative image of H3K9me3 and GFAP staining in AD+ mice that have been administered DMSO or ETP69.

FIG. 21L depicts a quantification of H3K9me3 staining in astrocytes of mice administered ETP69 or DMSO.

FIG. 22 depicts representative images of VGF, BDNF, and NCAM in 18-month AD+ mice.

FIG. 23 depicts an experimental protocol used.

FIG. 24A depicts open field test.

FIG. 24B depicts an analysis of rearing over time.

FIG. 24C depicts an analysis of average rearing for each treatment group.

FIG. 24D depicts a quantification of rearing for each treatment group.

FIG. 24E depicts an analysis of locomotor activity over time.

FIG. 24F depicts an analysis of average locomotor activity for each treatment group.

FIG. 24G depicts a quantification of locomotor activity for each treatment group.

FIG. 25A depicts a color visual-stimuli X-maze.

FIG. 25B depicts total number of entries in a test.

FIG. 25C depicts percent of alternations in treatment groups.

FIG. 25D depicts the progression of the percent alternation with entry for mice treated with ETP69 or oraB.

FIG. 25E depicts Kaplan-Meier curves for the percent of mice performing the first alternation versus entry.

FIG. 26A depicts a contrast visual-stimuli X-maze.

FIG. 26B depicts total number of entries in a test.

FIG. 26C depicts percent of alternations in treatment groups.

FIG. 26D depicts progression of percent alternation with entry for mice treated with ETP69 or oraB.

FIG. 26E depicts Kaplan-Meier curves for percent of mice performing a first alternation versus entry.

FIG. 27A depicts a Barnes maze test.

FIG. 27B depicts number of errors for each WT and AD+ mice administered oraB or ETP69.

FIG. 27C depicts the number of average number of errors for WT and AD+ mice administered oraB or ETP69 during the acquisition phase.

FIG. 27D depicts the number of average number of errors for WT and AD+ mice administered oraB or ETP69 during a memory retention phase

FIG. 27E depicts the number of average number of errors for WT and AD+ mice administered oraB or ETP69 during a reversal phase.

### DETAILED DESCRIPTION

There are over 5.5 million people living with Alzheimer's disease in the United States and tens of thousands with early-onset Alzheimer's disease. Alzheimer's disease may affect both the brain and retina, and patients may exhibit cognitive, behavioral and visual dysfunctions. Therapies are needed to prevent, slow down, or cure Alzheimer's disease and the cognitive and visual dysfunctions associated with Alzheimer's disease, and there is a need for effective treatments and the development of means for preserving cognitive function.

Cognitive decline is a devastating condition associated with neurodegenerative disorders and is dependent on aging in general. Early-onset Alzheimer's disease type present a remarkable challenge and a devastating condition by which carriers of certain mutations within amyloid precursor protein (APP), presenilin-1 (PSEN1), or presenilin-2 (PSEN2) genes may cause Alzheimer's disease with 100% penetrance. In some cases, this early-onset disease is derived from increased production of neurotoxic amyloid beta-protein and is associated with inflammation, vascular pathology, or other detrimental effects on histone methylation, gene expression and synaptic loss.

Epigenetic regulation of some synaptic proteins may be an underlying, yet reversible, cause of this decline. Histone 3 trimethylation is a likely target for pharmacological intervention that can counteract cognitive decline in the aging brain. Provided herein is evidence that, by manipulating an enzyme that regulates trimethylation of H3K9 (H3K9me3) (using ETP69, an inhibitor of SUV39H1), it is possible to alter the chromatin state of subjects and restore memory and synaptic function in the aging brain. Treatment with ETP69 may offer a unique mechanism for boosting gene expression and rejuvenating neuronal and synaptic activity. Treatment with ETP69 may prevent, slow down, or cure Alzheimer's disease and the cognitive and visual dysfunctions associated with Alzheimer's disease. Some advantages of ETP69 include an improved safety and toxicity profile, ready availability, and an ability to be safely prescribed.

Methylation of a histone tail typically occurs at specific lysine residues, such as H3K4, H3K9, H3K27, H3K36, H3K79 and H4K20, and may activate or repress transcription. Trimethylation of H3K9 (H3K9me3) may be a useful repressive histone mark, and is implicated in gene silencing. Establishment of H3K9me3 is affected by activity of the histone methyl transferase SUV39H1 which regulates H3K9 trimethylation at the peri-centric heterochromatin.

H3K9me3 may be a repressive histone mark, and is typically implicated in gene silencing. Some embodiments include a role for histone H3K9me3 and its histone methyl transferase (SUV39H1) in mediating hippocampal memory functions, and affecting Alzheimer's disease progression or development. Pharmacological inhibition of SUV39H1 using a selective inhibitor may decrease levels of H3K9me3 in the hippocampus of treated subjects, and/or improve performance in an object location memory task, a fear conditioning task or a complex spatial environment learning task. The inhibition of SUV39H1 by ETP69 or another compound disclosed herein may induce an increase in spine density of thin and stubby but not mushroom spines in the hippocampus of a treated subject, and increase GluR1-containing AMPA receptors levels at spine surface, a useful index of long-term potentiation (LTP). Establishment of H3K9me3 may depend on activity of the histone methyl transferase SUV39H1 which regulates H3K9 trimethylation at the peri-centric heterochromatin. Regulating the function of enzymes that contribute to histone methylation may hence be a powerful means to offset age-related cognitive deficits.

Disclosed herein, in some embodiments, are methods of treating, preventing or delaying onset of an Alzheimer's disease in a subject in need thereof, the method comprising administering to the subject a composition comprising ETP69. Disclosed herein, in some embodiments, are methods of improving cognition in a subject at risk of developing Alzheimer's disease, the method comprising administering to the subject a composition comprising ETP69.

Disclosed herein, in some embodiments, are methods of treating amyloidosis in a subject, the method comprising administering to the subject a composition comprising ETP69. The subject may subject have a mutation associated with familial Alzheimer's disease or a genetic risk factor associated with sporadic Alzheimer's disease. The administration may reduce the amyloidosis in the subject, relative to an amyloidosis measurement obtained before the administration.

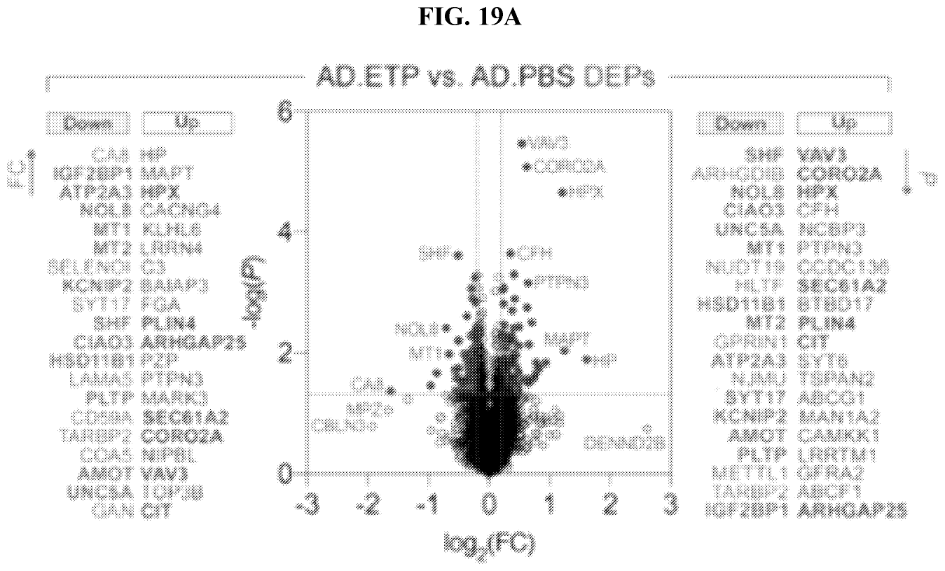

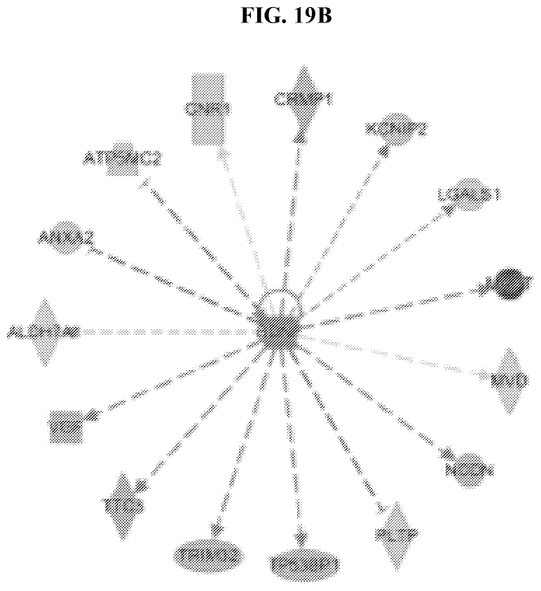

Disclosed herein, in some embodiments, are methods of modulating a molecular marker in a subject, the method comprising administering to the subject a composition comprising ETP69. For example, the method may include modulating (e.g. increasing or decreasing, relative to a baseline), one or more proteins as described in FIG. 19A, 19B or 20. The subject may subject have a mutation associated with familial Alzheimer's disease or a genetic risk factor associated with sporadic Alzheimer's disease.

Disclosed herein, in some embodiments, are methods of treating neuroinflammation in a subject, the method comprising administering to the subject a composition comprising ETP69. The subject may subject have a mutation associated with familial Alzheimer's disease or a genetic risk factor associated with sporadic Alzheimer's disease. The administration may reduce the neuroinflammation in the subject, relative to an amyloidosis measurement obtained before the administration.

A. Compounds

Provided herein, are compounds for use in methods of treating, preventing or delaying onset of Alzheimer's disease. In some embodiments, the compound inhibits a histone methyl transferase. In some embodiments, the compound inhibits SUV39H1. In some embodiments, the compound inhibits trimethylation of H3K9 (H3K9me3). For example, some embodiments include use of H3K9me3 modulation for enhancing cognitive function in aging and for treatment of age-related disorders such as Alzheimer's disease.

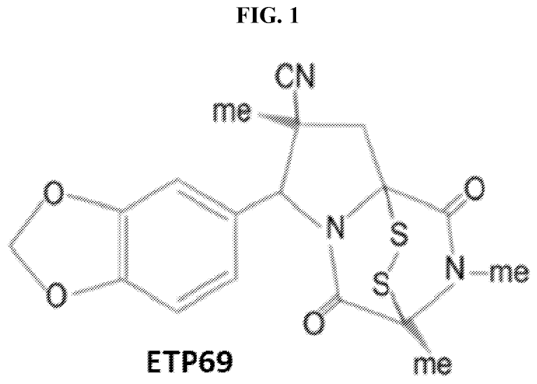

In some embodiments, the compound comprises ETP69 (Rac-(3S,6S,7S,8aS)-6-(benzo[d][1,3]dioxol-5-yl)-2,3,7-trimethyl-1,4-dioxohexahydro-6H-3, 8a-epidithiopyrrolo[1,2-a]pyrazine-7-carbonitrile). In some embodiments, the compound consists of ETP69. A structure of ETP69 is shown in FIG. 1. In some embodiments, the compound comprises ETP69, or a pharmaceutically acceptable salt thereof. In some embodiments, the compound consists of ETP69, or a pharmaceutically acceptable salt thereof.

In some embodiments, the compound includes an analog of ETP69. In some embodiments, the compound is an analog of ETP69. In some embodiments, the compound comprises or consists of an analog of ETP69, or a pharmaceutically acceptable salt thereof. In some embodiments, the analog of ETP69 is a compound having the following formula:

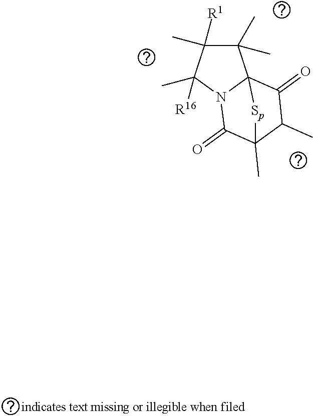

The symbol p may be 2, 3 or 4. In some embodiments, p is 2. In some embodiments, p is 3. In some embodiments, p is 4.

R¹, R², R³, R⁴, R⁵, R⁶, R¹⁶ and/or R¹⁸ may each independently be hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R¹ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R² is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R³ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R⁴ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R⁵ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R⁶ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R¹⁶ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R¹⁸ is hydrogen, a halogen, —N₃, —CF₃, —CCl₃, —CBr₃, —Cl₃, —CN, —CHO, —OH, —NH₂, —COH, —CONH₂, —NO₂, —SH, —SO₂, —SO₂Cl, —SO₃H, —SO₄H, —SO₂NH₂, —NHNH₂, —ONH₂, —NHC(O)NHNH₂, a substituted or unsubstituted alkyl, a substituted or unsubstituted heteroalkyl, a substituted or unsubstituted cycloalkyl, a substituted or unsubstituted heterocycloalkyl, a substituted or unsubstituted aryl, or a substituted or unsubstituted heteroaryl.

In some embodiments, R¹, R², R³, R⁴, R⁵, R⁶, R¹⁶, or R¹⁸ is hydrogen. In some embodiments, R¹, R², R³, R⁴, R⁵, R⁶, R¹⁶, or R¹⁸ is a halogen. In some embodiments, R¹, R², R³, R⁴, R⁵, R⁶, R¹⁶, or R¹⁸ is N₃. In some embodiments, R¹, R², R³, R⁴, R⁵, R⁶, R¹⁶, or R¹⁸ is —CF₃. In some embodiments, R¹, R², R³, R⁴, R⁵, R⁶, R¹⁶, or R¹⁸ is —CCl₃. In some embodiments, R¹, R², R³, R⁴, R⁵, R⁶, R¹⁶, or R¹⁸ is —CBr₃. In some embodiments, R¹, R², R³, R⁴, R⁵, R⁶, R¹⁶, or R¹⁸ is —Cl₃.

In some embodiments, R¹, R², R³, R⁴, R⁵, R⁶, R¹⁶, or R¹⁸ is CN. In some embodiments, R¹, R², R³, R⁴, R⁵, R⁶, R¹⁶, or R¹⁸ is CHO. In some embodiments, R¹, R², R³, R⁴, R⁵, R⁶, R¹⁶, or R¹⁸ is OH. In some embodiments, R¹, R², R³, R⁴, R⁵, R⁶, R¹⁶, or R¹⁸ is —NH₂. In some embodiments, R¹, R², R³, R⁴, R⁵, R⁶, R¹⁶, or R¹⁸ is —COH. In some embodiments, R, R³, R⁴, R⁵, R, R¹⁶, or R¹ is —CONH₂. In some embodiments, R¹, R², R³, R⁴, R⁵, R⁶, R¹⁶, or R¹⁸ is NO₂. In some embodiments, R¹, R², R³, R⁴, R⁵, R⁶, R¹⁶, or R¹⁸ is —SH. In some embodiments, R¹, R², R³, R⁴, R⁵, R⁶, R¹⁶, or R¹⁸ is —SO₂. In some embodiments, R¹, R², R³, R⁴, R⁵, R⁶, R¹⁶, or R¹⁸ is SO₂Cl. In some embodiments, R¹, R², R³, R⁴, R⁵, R⁶, R¹⁶, or R¹⁸ is —SO₃H. In some embodiments, R¹, R², R³, R⁴, R⁵, R⁶, R¹⁶, or R¹⁸ is SO₄H. In some embodiments, R¹, R², R³, R⁴, R⁵⁶, R⁶, R¹⁶, or R¹⁸ is SO₂NH₂. In some embodiments, R¹, R², R³, R⁴, R⁵, R⁶, R¹⁶, or R¹⁸ is —NHNH₂. In some embodiments, R¹, R², R³, R⁴, R⁵, R⁶, R¹⁶, or R¹⁸ is ONH₂. In some embodiments, R¹, R², R³, R⁴, R⁵, R⁶, R¹⁶, or R¹⁸ is —NHC(O)NHNH₂. In some embodiments, R¹, R², R³, R⁴, R⁵, R⁶, R¹⁶, or R¹⁸ is a substituted or unsubstituted alkyl. In some embodiments, R¹, R², R³, R⁴, R⁵, R⁶, R¹⁶, or R¹⁸ is a substituted or unsubstituted heteroalkyl. In some embodiments, R¹, R², R³, R⁴, R⁵, R⁶, R¹⁶, or R¹⁸ is a substituted or unsubstituted cycloalkyl. In some embodiments, R¹, R², R³, R⁴, R⁵, R⁶, R¹⁶, or R¹⁸ is a substituted or unsubstituted heterocycloalkyl. In some embodiments, R¹, R², R³, R⁴, R⁵, R⁶, R¹⁶, or R¹⁸ is a substituted or unsubstituted aryl. In some embodiments, R¹, R², R³, R⁴, R⁵, R⁶, R¹⁶, or R¹⁸ is a substituted or unsubstituted heteroaryl.

In some embodiments, the compound is used in the manufacture of a medicament for treatment of Alzheimer's disease. In some embodiments, the compound is used in the manufacture of a medicament for prevention of Alzheimer's disease. In some embodiments, the compound is used in the manufacture of a medicament for delaying onset of Alzheimer's disease.

B. Formulations

In certain embodiments, the compound as described herein is administered as a pure chemical. In other embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

C. Alzheimer's Disease Treatment and Prevention

Disclosed herein, in some embodiments, are methods of treating, preventing or delaying onset of an Alzheimer's disease in a subject in need thereof. Some embodiments include treating an Alzheimer's disease. Some embodiments include preventing an Alzheimer's disease. Some embodiments include delaying onset of an Alzheimer's disease. Some embodiments include treating or delaying onset of an Alzheimer's disease. Some embodiments include administering to the subject a composition described herein. For example, the composition may include ETP69 or a pharmaceutically acceptable salt thereof. In some embodiments, the subject has at least one mutation associated with familial Alzheimer's disease. In some embodiments, the subject has at least one genetic risk factor associated with sporadic Alzheimer's disease. In some embodiments, the subject has (i) at least one mutation associated with familial Alzheimer's disease; or (ii) at least one genetic risk factor associated with sporadic Alzheimer's disease. Some embodiments include administering to the subject a composition comprising ETP69 wherein the subject has (i) at least one mutation associated with familial Alzheimer's disease; or (ii) at least one genetic risk factor associated with sporadic Alzheimer's disease. Disclosed herein, in some embodiments, are methods of treating, preventing or delaying onset of an Alzheimer's disease in a subject in need thereof, the method comprising administering to the subject a composition comprising ETP69 wherein the subject has (i) at least one mutation associated with familial Alzheimer's disease; or (ii) at least one genetic risk factor associated with sporadic Alzheimer's disease.

Disclosed herein, in some embodiments, are methods of improving cognition in a subject. In some embodiments, the subject is risk of developing Alzheimer's disease. Some embodiments include improving cognition in a subject at risk of developing Alzheimer's disease. Some embodiments include administering to the subject a composition described herein. For example, the composition may include ETP69 or a pharmaceutically acceptable salt thereof. In some embodiments, the subject has at least one mutation associated with familial Alzheimer's disease. In some embodiments, the subject at least one genetic risk factor associated with sporadic Alzheimer's disease. In some embodiments, the subject has (i) at least one mutation associated with familial Alzheimer's disease; or (ii) at least one genetic risk factor associated with sporadic Alzheimer's disease. Some embodiments include administering to the subject a composition comprising ETP69 wherein the subject has (i) at least one mutation associated with familial Alzheimer's disease; or (ii) at least one genetic risk factor associated with sporadic Alzheimer's disease. Disclosed herein, in some embodiments, are methods of improving cognition in a subject at risk of developing Alzheimer's disease, the method comprising administering to the subject a composition comprising ETP69 wherein the subject has (i) at least one mutation associated with familial Alzheimer's disease; or (ii) at least one genetic risk factor associated with sporadic Alzheimer's disease.

Disclosed herein, in some embodiments, are methods of improving cognition in a subject in need thereof. Some embodiments include obtaining results of a genetic test. In some embodiments, the genetic test determines the presence of at least one mutation associated with familial Alzheimer's disease. In some embodiments, the results of the genetic test are for at least one mutation associated with familial Alzheimer's disease. The genetic test may be for any mutation or mutations described herein that are associated with familial Alzheimer's disease. Some embodiments include obtaining results of a genetic test for at least one mutation associated with familial Alzheimer's disease for the subject. In some embodiments, the genetic test indicates that the subject has at least one mutation associated with familial Alzheimer's disease. Some embodiments include administering to the subject a composition described herein. For example, the composition may include ETP69 or a pharmaceutically acceptable salt thereof. Some embodiments include administering the composition to the subject based on the results of the genetic test. Some embodiments include administering the composition to the subject when the genetic test indicates that the subject has at least one mutation associated with familial Alzheimer's disease. Some embodiments include administering a composition comprising ETP69 to the subject when the genetic test indicates that the subject has at least one mutation associated with familial Alzheimer's disease. Some embodiments include (a) obtaining results of a genetic test; and (b) administering a composition described herein to the subject based on the results of the genetic test. Some embodiments include (a) obtaining results of a genetic test for at least one mutation associated with familial Alzheimer's disease for the subject; and (b) administering a composition comprising ETP69 to the subject when the genetic test indicates that the subject has at least one mutation associated with familial Alzheimer's disease. Disclosed herein, in some embodiments, are methods of improving cognition in a subject in need thereof, the method comprising: (a) obtaining results of a genetic test for at least one mutation associated with familial Alzheimer's disease for the subject; and (b) administering a composition comprising ETP69 to the subject when the genetic test indicates that the subject has at least one mutation associated with familial Alzheimer's disease.

Disclosed herein, in some embodiments, are methods of improving cognition in a subject in need thereof. Some embodiments include obtaining results of a genetic test. In some embodiments, the genetic test determines the presence of at least one mutation associated with sporadic Alzheimer's disease. In some embodiments, the results of the genetic test are for at least one mutation associated with sporadic Alzheimer's disease. The genetic test may be for any mutation or mutations described herein that are associated with sporadic Alzheimer's disease. Some embodiments include obtaining results of a genetic test for at least one mutation associated with sporadic Alzheimer's disease for the subject. In some embodiments, the genetic test indicates that the subject has at least one mutation associated with sporadic Alzheimer's disease. Some embodiments include administering to the subject a composition described herein. For example, the composition may include ETP69 or a pharmaceutically acceptable salt thereof. Some embodiments include administering the composition to the subject based on the results of the genetic test. Some embodiments include administering the composition to the subject when the genetic test indicates that the subject has at least one mutation associated with sporadic Alzheimer's disease. Some embodiments include administering a composition comprising ETP69 to the subject when the genetic test indicates that the subject has at least one mutation associated with sporadic Alzheimer's disease. Some embodiments include (a) obtaining results of a genetic test; and (b) administering a composition described herein to the subject based on the results of the genetic test. Some embodiments include (a) obtaining results of a genetic test for at least one mutation associated with sporadic Alzheimer's disease for the subject; and (b) administering a composition comprising ETP69 to the subject when the genetic test indicates that the subject has at least one mutation associated with sporadic Alzheimer's disease. Disclosed herein, in some embodiments, are methods of improving cognition in a subject in need thereof, the method comprising: (a) obtaining results of a genetic test for at least one mutation associated with sporadic Alzheimer's disease for the subject; and (b) administering a composition comprising ETP69 to the subject when the genetic test indicates that the subject has at least one mutation associated with sporadic Alzheimer's disease.

D. Administration

In some embodiments, administering the compound (e.g. ETP69) to the subject comprises administering an effective amount of the compound sufficient to inhibit SUV39H1 in the subject. In some embodiments, the route of administration is intravenous, oral, subcutaneous, intraperitoneal, ocular, intraocular, intramuscular, interstitial, or intracranial. In some embodiments, the administration is systemic. In some embodiments, the administration is intravenous. In some embodiments, the administration is oral. In some embodiments, the administration comprises an injection. In some embodiments, the administration is subcutaneous. In some embodiments, the administration is intraperitoneal. In some embodiments, the administration is ocular. In some embodiments, the administration is intraocular. In some embodiments, the administration is intramuscular. In some embodiments, the administration is interstitial. In some embodiments, the administration is intracranial.

In some embodiments, administering the compound comprises administering a single dose. In some embodiments, administering the compound comprises administering multiple doses (for example, 2 doses). For example, the administration may include multiple doses at separate times. The multiple doses may include 1, 2, 3, 4, 5, 6, 7, 8, 9, 10, 11, 12, 13, 14, 15, 16, 17, 18, 19, or 20, or more doses, or a range of doses defined by any two of the aforementioned numbers of doses. In some embodiments, the administration includes administering 11 doses.

E. Alzheimer's Disease and Mutations

Disclosed herein, in some embodiments, are methods of treating, preventing or delaying onset of an Alzheimer's disease in a subject. In some embodiments, the Alzheimer's disease comprises early-onset Alzheimer's disease. In some embodiments, the Alzheimer's disease comprises late-onset Alzheimer's disease. In some embodiments, the Alzheimer's disease is familial. In some embodiments, the Alzheimer's disease comprises familial Alzheimer's disease. In some embodiments, the early-onset Alzheimer's disease comprises familial Alzheimer's disease. In some embodiments, the Alzheimer's disease is sporadic. In some embodiments, the Alzheimer's disease comprises sporadic Alzheimer's disease.

Some embodiments relate to a symptom of Alzheimer's disease. In some embodiments, the symptom comprises memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes. In some embodiments, the symptom includes memory loss. In some embodiments, the symptom includes difficulty concentrating. In some embodiments, the symptom includes difficulty completing familiar tasks. In some embodiments, the symptom includes confusion with time or place. In some embodiments, the symptom includes difficulty understanding visual images and spatial relationships. In some embodiments, the symptom includes language difficulties. In some embodiments, the symptom includes misplacing items. In some embodiments, the symptom includes decreased or poor judgement. In some embodiments, the symptom includes social withdrawal. In some embodiments, the symptom includes mood or personality changes.

Disclosed herein, in some embodiments, are methods of treating, preventing or delaying onset of a familial Alzheimer's disease. In some embodiments, the familial Alzheimer's disease comprises Alzheimer's disease type 1. In some embodiments, the familial Alzheimer's disease comprises Alzheimer's disease type 3. In some embodiments, the familial Alzheimer's disease comprises Alzheimer's disease type 4.

In some embodiments, the subject has a mutation associated with the Alzheimer's disease (e.g. familial or sporadic Alzheimer's disease). In some embodiments, the mutation is recessive. In some embodiments, the mutation is dominant. In some embodiments, the mutation is heterozygous. In some embodiments, the mutation is homozygous.

In some embodiments, the subject has 1 mutation associated with the Alzheimer's disease. In some embodiments, the subject has 2 mutations associated with the Alzheimer's disease. In some embodiments, the subject has 3 mutations associated with the Alzheimer's disease. In some embodiments, the subject has 4 mutations associated with the Alzheimer's disease. In some embodiments, the subject has 5 mutations associated with the Alzheimer's disease. In some embodiments, the subject has 1, 2, 3, 4, 5, 6, 7, 8, 9, 10, 11, 12, 13, 14, or 15 mutations associated with the Alzheimer's disease. In some embodiments, the subject has 1, 2, 3, 4, 5, 6, 7, 8, 9, 10, 11, 12, 13, 14, or 15 mutations associated with the Alzheimer's disease, or a range of mutations defined by any two of the aforementioned integers.

In some embodiments, the subject has at least 1 mutation associated with the Alzheimer's disease. In some embodiments, the subject has at least 2 mutations associated with the Alzheimer's disease. In some embodiments, the subject has at least 3 mutations associated with the Alzheimer's disease. In some embodiments, the subject has at least 4 mutations associated with the Alzheimer's disease. In some embodiments, the subject has at least 5 mutations associated with the Alzheimer's disease. In some embodiments, the subject has at least 1, at least 2, at least 3, at least 4, at least 5, at least 6, at least 7, at least 8, at least 9, at least 10, at least 11, at least 12, at least 13, at least 14, or at least 15 mutations associated with the Alzheimer's disease.

In some embodiments, the subject has no more than 1 mutation associated with the Alzheimer's disease. In some embodiments, the subject has no more than 2 mutations associated with the Alzheimer's disease. In some embodiments, the subject has no more than 3 mutations associated with the Alzheimer's disease. In some embodiments, the subject has no more than 4 mutations associated with the Alzheimer's disease. In some embodiments, the subject has no more than 5 mutations associated with the Alzheimer's disease. In some embodiments, the subject has no more than 1, no more than 2, no more than 3, no more than 4, no more than 5, no more than 6, no more than 7, no more than 8, no more than 9, no more than 10, no more than 11, no more than 12, no more than 13, no more than 14, or no more than 15 mutations associated with the Alzheimer's disease.

In some embodiments, the subject has at least one mutation associated with familial Alzheimer's disease. In some embodiments, the at least one mutation associated with familial Alzheimer's disease comprises a mutation in an amyloid precursor protein (APP) gene, a presenilin-1 (PSEN1) gene, or a presenilin-2 (PSEN2) gene. In some embodiments, the at least one mutation associated with familial Alzheimer's disease comprises a mutation in an amyloid precursor protein (APP) gene. In some embodiments, the at least one mutation associated with familial Alzheimer's disease comprises a mutation in a presenilin-1 (PSEN1) gene. In some embodiments, the at least one mutation associated with familial Alzheimer's disease comprises a mutation in a presenilin-2 (PSEN2) gene.

In some embodiments, the mutation in the APP gene codes for a mutation in the amyloid precursor protein amino acid sequence that is expressed from the APP gene. In some embodiments, the mutation in the amyloid precursor protein amino acid sequence comprises a mutation at lysine 670 according to SEQ ID NO: 1. In some embodiments, the lysine 670 is mutated to asparagine (K670N). In some embodiments, the mutation in the amyloid precursor protein amino acid sequence comprise a mutation at methionine 671 according to SEQ ID NO: 1. In some embodiments, the methionine 671 is mutated to lysine (M671L). In some embodiments, the mutation in the amyloid precursor protein amino acid sequence comprises mutations at positions lysine 670 and methionine 671 according to SEQ ID NO: 1. In some embodiments, the lysine 670 and methionine 671 are mutated to asparagine and lysine, respectively (KM670/671NL, Swedish mutation).

In some embodiments, the mutation in the amyloid precursor protein amino acid sequence comprises a mutation at alanine 673 according to SEQ ID NO: 1. In some embodiments, the alanine 673 of the amyloid precursor protein is mutated to valine (A673V).

In some embodiments, the mutation in the amyloid precursor protein amino acid sequence comprises a mutation at histidine 677 according to SEQ ID NO: 1. In some embodiments, the histidine 677 of the amyloid precursor protein is mutated to arginine (H677R).

In some embodiments, the mutation in the amyloid precursor protein amino acid sequence comprises a mutation at aspartate 678 according to SEQ ID NO: 1. In some embodiments, the aspartate 678 of the amyloid precursor protein is mutated to asparagine (D678N).

In some embodiments, the mutation in the amyloid precursor protein amino acid sequence comprises a mutation at alanine 692 according to SEQ ID NO: 1. In some embodiments, the alanine 692 of the amyloid precursor protein is mutated to glycine (A692G).

In some embodiments, the mutation in the amyloid precursor protein amino acid sequence comprises a mutation at glutamate 693 according to SEQ ID NO: 1. In some embodiments, the mutation in the APP gene codes for a deletion at position glutamate 693 of the amyloid precursor protein amino acid (E693A, Osaka mutation). In some embodiments, the glutamate 693 of the of the amyloid precursor protein is mutated to glutamine (E693Q), glycine (E693G), or lysine (E693K). In some embodiments, the glutamate 693 of the of the amyloid precursor protein is mutated to glutamine (E693Q, Dutch mutation). In some embodiments, the glutamate 693 of the of the amyloid precursor protein is mutated to glycine (E693G, Arctic mutation). In some embodiments, the glutamate 693 of the of the amyloid precursor protein is mutated to lysine (E693K, Italian mutation).

In some embodiments, the mutation in the amyloid precursor protein amino acid sequence comprises a mutation at aspartate 694 according to SEQ ID NO: 1. In some embodiments, the aspartate 694 of the amyloid precursor protein is mutated to asparagine (D694N, Iowa mutation).

In some embodiments, the mutation in the amyloid precursor protein amino acid sequence comprises a mutation at alanine 713 according to SEQ ID NO: 1. In some embodiments, the alanine 713 of the amyloid precursor protein is mutated to threonine (A713T).

In some embodiments, the mutation in the amyloid precursor protein amino acid sequence comprises a mutation at threonine 714 according to SEQ ID NO: 1. In some embodiments, the threonine 714 of the of the amyloid precursor protein is mutated to isoleucine (T714I) or alanine (T714A). In some embodiments, the threonine 714 of the of the amyloid precursor protein is mutated to isoleucine (T714I, Austrian mutation). In some embodiments, the threonine 714 of the of the amyloid precursor protein is mutated to alanine (T714A, Iranian mutation).

In some embodiments, the mutation in the amyloid precursor protein amino acid sequence comprises a mutation at valine 715 according to SEQ ID NO: 1. In some embodiments, the valine 715 of the of the amyloid precursor protein is mutated to methionine (V715M) or alanine (V715A). In some embodiments, the valine 715 of the of the amyloid precursor protein is mutated to methionine (V715M, French mutation). In some embodiments, the valine 715 of the of the amyloid precursor protein is mutated to alanine (V715A, German mutation).

In some embodiments, the mutation in the amyloid precursor protein amino acid sequence comprises a mutation at isoleucine 716 according to SEQ ID NO: 1. In some embodiments, the isoleucine 716 of the of the amyloid precursor protein is mutated to valine (I716V) or phenylalanine (I716F). In some embodiments, the isoleucine 716 of the of the amyloid precursor protein is mutated to valine (I716V). In some embodiments, the isoleucine 716 of the of the amyloid precursor protein is mutated to phenylalanine (I716F, Florida mutation).

In some embodiments, the mutation in the amyloid precursor protein amino acid sequence comprises a mutation at valine 717 according to SEQ ID NO: 1. In some embodiments, the valine 717 of the amyloid precursor protein is mutated to isoleucine (V717I), phenylalanine (V717F), glycine (V717G), or leucine (V717L). In some embodiments, the valine 717 of the amyloid precursor protein is mutated to isoleucine (V717I, London mutation). In some embodiments, the valine 717 of the amyloid precursor protein is mutated to phenylalanine (V717F, Indiana mutation). In some embodiments, the valine 717 of the amyloid precursor protein is mutated to glycine (V717G). In some embodiments, the valine 717 of the amyloid precursor protein is mutated to leucine (V717L).

In some embodiments, the mutation in the amyloid precursor protein amino acid sequence comprises a mutation at threonine 719 according to SEQ ID NO: 1. In some embodiments, the threonine 719 of the amyloid precursor protein is mutated to proline (T719P).

In some embodiments, the mutation in the amyloid precursor protein amino acid sequence comprises a mutation at leucine 723 according to SEQ ID NO: 1. In some embodiments, the leucine 723 of the amyloid precursor protein is mutated to proline (L723P).

In some embodiments, the mutation in the amyloid precursor protein amino acid sequence comprises a mutation at lysine 724 according to SEQ ID NO: 1. In some embodiments, the lysine 724 of the amyloid precursor protein is mutated to asparagine (K724N).

In some embodiments, the mutation in the PSEN-1 gene codes for a mutation in the presenilin-1 amino acid sequence that is expressed from the PSEN-1 gene. In some embodiments, the mutation in the presenilin-1 amino acid sequence comprises a mutation at methionine 146 according to SEQ ID NO: 2. In some embodiments, the methionine 146 is mutated to leucine (M146L). In some embodiments, the mutation in the presenilin-1 amino acid sequence comprises a mutation at leucine 166 according to SEQ ID NO: 2. In some embodiments, the leucine 166 is mutated to proline (L166P). In some embodiments, the mutation in the presenilin-1 amino acid sequence comprises a mutation at isoleucine 213 according to SEQ ID NO: 2. In some embodiments, the isoleucine 213 is mutated to threonine (I213T). In some embodiments, the mutation in the presenilin-1 amino acid sequence comprises a mutation at arginine 278 according to SEQ ID NO: 2. In some embodiments, the arginine 278 is mutated to isoleucine (R278I). In some embodiments, the mutation in the presenilin-1 amino acid sequence comprises a mutation at alanine 246 according to SEQ ID NO: 2. In some embodiments, the alanine 246 is mutated to glutamate (A246E). In some embodiments, the mutation in the presenilin-1 amino acid sequence comprises a mutation at methionine 146, leucine 166, isoleucine 213, arginine 278, or alanine 246 according to SEQ ID NO: 2. In some embodiments, the methionine 146 is mutated to leucine (M146L), the leucine 166 is mutated to proline (L166P), the isoleucine 213 is mutated to threonine (I213T), the arginine 278 is mutated to isoleucine (R278I), or the alanine 246 is mutated to glutamate (A246E). In some embodiments, the methionine 146 is mutated to leucine (M146L), the leucine 166 is mutated to proline (L166P), the isoleucine 213 is mutated to threonine (I213T), the arginine 278 is mutated to isoleucine (R278I), and the alanine 246 is mutated to glutamate (A246E).

In some embodiments, the mutation in the PSEN-2 gene codes for a mutation in the presenilin-2 amino acid sequence that is expressed from the PSEN-2 gene. In some embodiments, the mutation in the presenilin-2 amino acid sequence comprises a mutation at asparagine 141 or methionine 239 according to SEQ ID NO: 3. In some embodiments, the asparagine 141 is mutated to isoleucine (N141I) and the methionine 239 is mutated to valine (M239V). In some embodiments, the mutation in the presenilin-2 amino acid sequence comprises a mutation at asparagine 141 according to SEQ ID NO: 3. In some embodiments, the asparagine 141 is mutated to isoleucine (N141I). In some embodiments, the mutation in the presenilin-2 amino acid sequence comprises a mutation at methionine 239 according to SEQ ID NO: 3. In some embodiments, the methionine 239 is mutated to valine (M239V).

In some embodiments, the subject has at least one mutation associated with sporadic Alzheimer's disease. In some embodiments, the at least one mutation associated with sporadic Alzheimer's disease comprises a mutation in an apolipoprotein. In some embodiments, the at least one mutation associated with sporadic Alzheimer's disease comprises a mutation in apolipoprotein e4. In some embodiments, the genetic risk factor associated with sporadic Alzheimer's disease comprises the subject being a carrier of an apolipoprotein (APOE) e4 allele.

In some embodiments, the genetic risk factor associated with sporadic Alzheimer's disease comprises a mutation in a gene. In some embodiments, the gene comprises ABCA7, AKAP9, BIN1, CASS4, CD2AP, CD33, CLU, EPHA1, FERMT2, HLA-DRB5/DRB1, INPP5D, MEF2C, MS4A6A/MS4A4E, PICALM, PLD3, PTK2B, —SORL1, ACE, TREM2, or UNC5C. In some embodiments, the gene comprises ABCA7 (ATP Binding Cassette Subfamily A Member 7). In some embodiments, the gene comprises AKAP9 (A-Kinase Anchoring Protein 9). In some embodiments, the gene comprises BIN1 (Bridging Integrator 1). In some embodiments, the gene comprises CASS4 (Cas Scaffold Protein Family Member 4). In some embodiments, the gene comprises CD2AP (CD2 Associated Protein). In some embodiments, the gene comprises CD33 (sialic acid binding Ig-like lectin 3). In some embodiments, the gene comprises CLU(Clusterin). In some embodiments, the gene comprises EPHA1 (ephrin type-A receptor 1). In some embodiments, the gene comprises FERMT2 (Fermitin Family Member 2). In some embodiments, the gene comprises HLA-DRB5/DRB1 (Major Histocompatibility Complex, Class II, DR Beta 5). In some embodiments, the gene comprises INPP5D (inositol polyphosphate-5-phosphatase). In some embodiments, the gene comprises MEF2C (Myocyte Enhancer Factor 2C). In some embodiments, the gene comprises MS4A6A/MS4A4E (Membrane Spanning 4-Domains A6A/Membrane Spanning 4-Domains A4E). In some embodiments, the gene comprises PICALM(Phosphatidylinositol Binding Clathrin Assembly Protein). In some embodiments, the gene comprises PLD3 (phospholipase D). In some embodiments, the gene comprises ACE (angiotensin-converting enzyme). In some embodiments, the gene comprises PTK2B (Protein tyrosine kinase 2 beta). In some embodiments, the gene comprises SORL1 (Sortilin Related Receptor 1). In some embodiments, the gene comprises TREM2 (Triggering Receptor Expressed On Myeloid Cells 2). In some embodiments, the gene comprises UNC5C (Unc-5 Netrin Receptor C).

In some embodiments, multiple genes associated with sporadic Alzheimer's disease are mutated in the subject.

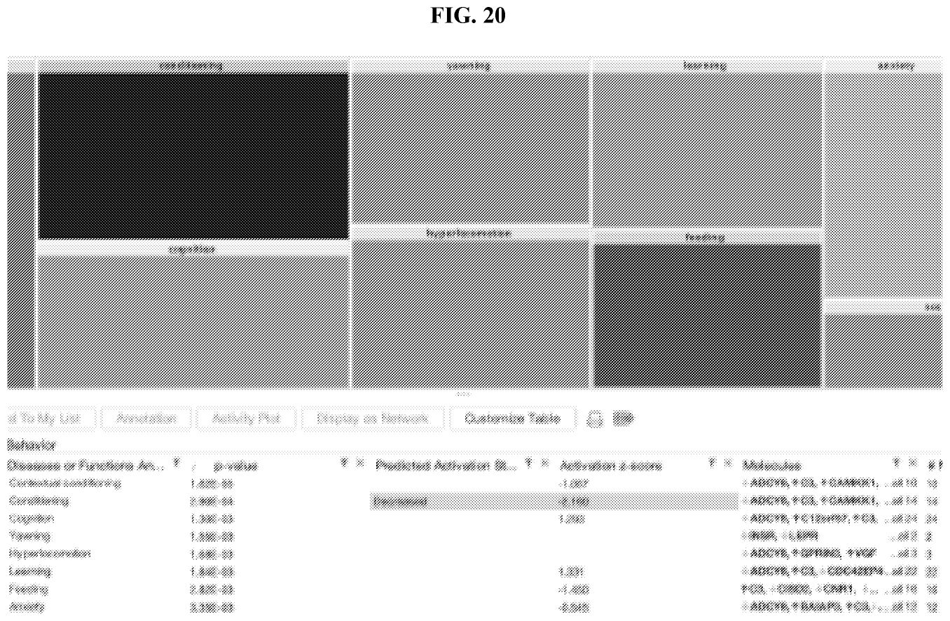

In some embodiments, the subject has altered expression of one or more proteins in FIG. 19A, relative to a control subject. In some embodiments, the subject has altered expression of one or more proteins in FIG. 19B, relative to a control subject. In some embodiments, the subject has altered expression of one or more proteins in FIG. 20, relative to a control subject. The control subject may be a subject without Alzheimer's disease, or may be a subject without a genetic risk factor associated with sporadic Alzheimer's disease.

F. Subjects

Some embodiments of the methods described herein include administration of a compound to a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female.

In some embodiments, the subject is ≥90 years of age. In some embodiments, the subject is ≥85 years of age. In some embodiments, the subject is ≥80 years of age. In some embodiments, the subject is ≥70 years of age. In some embodiments, the subject is ≥60 years of age. In some embodiments, the subject is ≥50 years of age. In some embodiments, the subject is ≥40 years of age. In some embodiments, the subject is ≥30 years of age. In some embodiments, the subject is ≥20 years of age. In some embodiments, the subject is ≥10 years of age. In some embodiments, the subject is ≥1 years of age. In some embodiments, the subject is ≥0 years of age. In some embodiments, the subject is asymptomatic of Alzheimer's disease. In some embodiments, the subject is at least about 25, about 30, about 35, about 40, about 45, about 50, about, about 55, about 60, about 65, or about 70 years of age.

In some embodiments, the subject is ≤100 years of age. In some embodiments, the subject is ≤90 years of age. In some embodiments, the subject is ≤85 years of age. In some embodiments, the subject is ≤80 years of age. In some embodiments, the subject is ≤70 years of age. In some embodiments, the subject is ≤60 years of age. In some embodiments, the subject is ≤50 years of age. In some embodiments, the subject is 40 years of age. In some embodiments, the subject is ≤30 years of age. In some embodiments, the subject is ≤20 years of age. In some embodiments, the subject is ≤10 years of age. In some embodiments, the subject is ≤1 years of age. In some embodiments, the subject is less than about 25, about 30, about 35, about 40, about 45, about 50, about, about 55, about 60, about 65, or about 70 years of age.

In some embodiments, the subject is between 0 and 100 years of age. In some embodiments, the subject is between 20 and 90 years of age. In some embodiments, the subject is between 30 and 80 years of age. In some embodiments, the subject is between 40 and 75 years of age. In some embodiments, the subject is between 50 and 70 years of age. In some embodiments, the subject is between 40 and 85 years of age.

In some embodiments, the subject has Alzheimer's disease. In some embodiments, the subject is at risk of developing Alzheimer's disease. The subject may subject have a mutation associated with familial Alzheimer's disease or a genetic risk factor associated with sporadic Alzheimer's disease. In some embodiments, the subject is symptomatic for the Alzheimer's disease. In some embodiments, the subject is asymptomatic of Alzheimer's disease.

G. Baseline Characteristics

Some embodiments of the methods described herein include obtaining a baseline measurement from a subject. For example, in some embodiments, a baseline measurement is obtained from the subject prior to treating the subject. In some embodiments, the baseline measurement is a symptom of the Alzheimer's disease such as a symptom described herein. Non-limiting examples of baseline measurements include a baseline memory measurement, a baseline learning measurement, a baseline spontaneous activity measurement, a baseline neuronal architecture measurement, a baseline neuroinflammation measurement, a baseline amyloidosis measurement, or a baseline biomarker measurement.

In some embodiments, the baseline measurement includes a mental status test. In some embodiments, the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test. In some embodiments, the mental status test is a Mini-Mental State Exam (MMSE). In some embodiments, the mental status test is a Mini-Cog test. In some embodiments, the mental status test is a Cantab Mobile test. In some embodiments, the mental status test is a Cognigram test. In some embodiments, the mental status test is a Cognivue test. In some embodiments, the mental status test is a Cognision and Automated Neuropsychological Assessment Metrics (ANAM) test. In some embodiments, the baseline measurement includes a neuroimaging test. In some embodiments, the neuroimaging test is a magnetic resonance imaging (MRI) or computed tomography (CT). In some embodiments, the neuroimaging test is an MRI test. In some embodiments, the neuroimaging test is a CT test.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device. In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, or a fluorescence assay. In some embodiments, the baseline measurement is obtained by PCR.

In some embodiments, the baseline measurement is a baseline memory measurement. In some embodiments, the baseline memory measurement is a baseline spatial memory measurement. In some embodiments, the baseline memory measurement is a baseline hippocampal-based spatial memory measurement. In some embodiments, the baseline spatial memory measurement comprises a fear-based test. In some embodiments, the baseline spatial memory measurement comprises a fear-conditioning test.

In some embodiments, the baseline measurement is a baseline learning measurement. In some embodiments, the baseline learning measurement is a baseline visual-cognitive memory and learning measurement. In some embodiments, the baseline visual-cognitive memory and learning measurement includes a color memory and learning measurement. In some embodiments, the baseline visual-cognitive memory and learning measurement includes a contrast memory and learning measurement. In some embodiments, the baseline visual-cognitive memory and learning measurement includes a transition memory and learning measurement. In some embodiments, the baseline visual-cognitive memory and learning measurement includes a spatial memory and learning measurement. In some embodiments, the baseline measurement is a baseline spontaneous activity measurement.

In some embodiments, the baseline measurement is a baseline neuronal architecture measurement. In some embodiments, the baseline neuronal architecture measurement includes a baseline spine integrity measurement. In some embodiments, the baseline neuronal architecture measurement includes a baseline dendritic spine measurement. In some embodiments, the baseline dendritic spine measurement assesses long-thin filopodia, long thin, thin, stubby, wide-headed mushroom, and/or branched spines. In some embodiments, the baseline neuronal architecture measurement includes a baseline spine density measurement. In some embodiments, the baseline neuronal architecture measurement includes a number of synapses. In some embodiments, the baseline neuronal architecture measurement is determined in a biopsy. In some embodiments, the baseline neuronal architecture measurement is determined using a stain such as a Golgi-Cox stain. In some embodiments, the baseline neuronal architecture measurement is determined using photography. In some embodiments, the baseline neuronal architecture measurement is determined using microscopy.

In some embodiments, the baseline measurement includes a baseline neuroinflammation measurement. In some embodiments, the baseline neuroinflammation measurement includes a baseline activated or a baseline reactive immune activation measurement. In some embodiments, the baseline neuroinflammation measurement includes a baseline activated or a baseline reactive immune cell measurement. In some embodiments, the baseline neuroinflammation measurement includes a baseline reactive astrocyte measurement. In some embodiments, the baseline neuroinflammation measurement includes a baseline activated microglia measurement. In some embodiments, the baseline neuroinflammation measurement includes a baseline macrophage measurement. In some embodiments, the baseline neuroinflammation measurement is obtained in a tissue or fluid sample. In some embodiments, the baseline neuroinflammation measurement is obtained from a biopsy. In some embodiments, the baseline neuroinflammation measurement is obtained by an assay such as an immunoassay, by fluorescence-activated Cell Sorting (FACS), or by histological assessment.

In some embodiments, the baseline measurement is a baseline amyloidosis measurement. In some embodiments, the baseline amyloidosis measurement includes a baseline amyloid plaque measurement. In some embodiments, the baseline amyloidosis measurement includes a baseline amyloid beta (Aβ) measurement. In some embodiments, the baseline Aβ measurement includes a baseline Aβ₁₋₄₂ measurement. In some embodiments, the baseline Aβ measurement includes a baseline soluble Aβ measurement. In some embodiments, the baseline Aβ measurement includes a baseline soluble Aβ₁₋₄₂ measurement. The baseline amyloidosis measurement may include a baseline central nervous system (CNS) amyloidosis measurement. The baseline amyloidosis measurement may include a v baseline vascular amyloidosis measurement. In some embodiments, the baseline amyloidosis measurement includes a baseline concentration or amount. The baseline amyloidosis measurement (e.g. the baseline amyloid plaque measurement) may be performed using an imaging device. The imaging device may include a positron emission tomography (PET) device. The baseline amyloidosis measurement may be performed on a biopsy. The baseline amyloidosis measurement may be performed using a spinal tap (for example, when the baseline amyloidosis measurement includes a baseline cerebrospinal fluid (CSF) amyloidosis measurement). In some embodiments, the baseline amyloidosis measurement is obtained by an assay such as an immunoassay.

In some embodiments, the baseline measurement is a baseline molecular marker measurement. In some embodiments, the baseline molecular marker measurement is a baseline histone trimethylation (H3K9me3) measurement. In some embodiments, the baseline molecular marker measurement is a baseline protein measurement. In some embodiments, the baseline molecular marker measurement is a baseline brain-derived neurotrophic factor (BDNF) measurement. In some embodiments, the baseline molecular marker measurement is a baseline Aβ measurement. In some embodiments, the baseline measurement includes a beta-amyloid deposit measurement. In some embodiments, the baseline protein measurement is a baseline measurement of a protein in FIG. 19A. In some embodiments, the baseline protein measurement is a baseline measurement of a protein in FIG. 19B. In some embodiments, the baseline measurement is a baseline measurement of an aspect or protein in FIG. 20. In some embodiments, the baseline molecular marker measurement is determined in a biopsy. In some embodiments, the baseline molecular marker measurement is determined using an immunoassay such as an ELISA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject.

In some embodiments, the sample comprises a fluid. In some embodiments, the sample is a fluid sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. In some embodiments, the sample comprises serum. In some embodiments, the sample is a serum sample. In some embodiments, the sample comprises cerebrospinal fluid (CSF).

In some embodiments, the sample comprises a tissue. In some embodiments, the sample is a tissue sample. In some embodiments, the sample comprises neural tissue. In some embodiments, the sample is a brain sample. In some embodiments, the sample is a hippocampal sample. In some embodiments, the sample comprises neurons.

In some embodiments, the sample comprises cells. The cells may include neural cells. The cells may include cerebral cells. The cells may include cerebral macrophages, microglia, or astrocytes. In some embodiments, the cells include neurons. In some embodiments, the cells include macrophages. In some embodiments, the cells include microglia. In some embodiments, the cells include astrocytes.

H. Treatment Effects

In some embodiments, the composition or administration of the composition affects a measurement such as a memory measurement, a learning measurement, a spontaneous activity measurement, a neuronal architecture measurement, a neuroinflammation measurement, an amyloidosis measurement, or a biomarker measurement, relative to the baseline measurement. In some embodiments, the measurement is related to a symptom of the Alzheimer's disease.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, or a PCR assay. In some embodiments, the measurement is obtained by an assay such as an immunoassay, a colorimetric assay, or a fluorescence assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. In some embodiments, the reduction is measured in a second tissue sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages.

Some embodiments of the methods described herein include obtaining the measurement from a

In some embodiments, the composition increases the measurement relative to the baseline measurement. In some embodiments, the increase is measured in a second tissue sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement includes a mental status test. In some embodiments, the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test. In some embodiments, the measurement includes a neuroimaging test. In some embodiments, the neuroimaging test is a magnetic resonance imaging (MRI) or computed tomography (CT).

In some embodiments, the measurement is a memory measurement. In some embodiments, the memory measurement is a spatial memory measurement. In some embodiments, the memory measurement is a hippocampal-based spatial memory measurement. In some embodiments, the spatial memory measurement comprises a fear-based test. In some embodiments, the spatial memory measurement comprises a fear-conditioning test. In some embodiments, the memory measurement is improved. In some embodiments, the memory measurement is increased.

In some embodiments, the measurement is a learning measurement. In some embodiments, the learning measurement is a visual-cognitive memory and learning measurement. In some embodiments, the visual-cognitive memory and learning measurement includes a color memory and learning measurement. In some embodiments, the visual-cognitive memory and learning measurement includes a contrast memory and learning measurement. In some embodiments, the visual-cognitive memory and learning measurement includes a transition memory and learning measurement. In some embodiments, the visual-cognitive memory and learning measurement includes a spatial memory and learning measurement. In some embodiments, the learning measurement is improved. In some embodiments, the learning measurement is increased. In some embodiments, the measurement is a spontaneous activity measurement. In some embodiments, the spontaneous activity measurement is improved. In some embodiments, the spontaneous activity measurement is increased.

In some embodiments, the measurement is a neuronal architecture measurement. In some embodiments, the neuronal architecture measurement includes a spine integrity measurement. In some embodiments, the neuronal architecture measurement includes a dendritic spine measurement. In some embodiments, the dendritic spine measurement assesses long-thin filopodia, long thin, thin, stubby, wide-headed mushroom, and/or branched spines. In some embodiments, the neuronal architecture measurement includes a spine density measurement. In some embodiments, the neuronal architecture measurement includes a number of synapses. In some embodiments, the neuronal architecture measurement is determined in a biopsy. In some embodiments, the neuronal architecture measurement is determined using a stain such as a Golgi-Cox stain. In some embodiments, the neuronal architecture measurement is determined using photography. In some embodiments, the neuronal architecture measurement is determined using microscopy. In some embodiments, the neuronal architecture measurement is improved. In some embodiments, the neuronal architecture measurement (e.g. number of synapses) is increased.

In some embodiments, the measurement includes a neuroinflammation measurement. In some embodiments, the neuroinflammation measurement includes an activated or a reactive immune activation measurement. In some embodiments, the neuroinflammation measurement includes an activated or a reactive immune cell measurement. In some embodiments, the neuroinflammation measurement includes a reactive astrocyte measurement. In some embodiments, the neuroinflammation measurement includes an activated microglia measurement. In some embodiments, the neuroinflammation measurement includes a macrophage measurement. In some embodiments, the neuroinflammation measurement is obtained in a tissue or fluid sample. In some embodiments, the neuroinflammation measurement is obtained from a biopsy. In some embodiments, the neuroinflammation measurement is obtained by an assay such as an immunoassay, by fluorescence-activated Cell Sorting (FACS), or by histological assessment.

In some embodiments, the measurement is an amyloidosis measurement. In some embodiments, the amyloidosis measurement includes an amyloid plaque measurement. In some embodiments, the amyloidosis measurement includes an amyloid beta (Aβ) measurement. In some embodiments, the Aβ measurement includes an Aβ₁₋₄₂ measurement. In some embodiments, the Aβ measurement includes a soluble Aβ measurement. In some embodiments, the Aβ measurement includes a soluble Aβ₁₋₄₂ measurement. The amyloidosis measurement may include a central nervous system (CNS) amyloidosis measurement. The amyloidosis measurement may include a vascular amyloidosis measurement. In some embodiments, the amyloidosis measurement includes a concentration or amount. The amyloidosis measurement (e.g. the amyloid plaque measurement) may be performed using an imaging device. The imaging device may include a positron emission tomography (PET) device. The amyloidosis measurement may be performed on a biopsy. The amyloidosis measurement may be performed using a spinal tap (for example, when the amyloidosis measurement includes a cerebrospinal fluid (CSF) amyloidosis measurement). In some embodiments, the amyloidosis measurement is obtained by an assay such as an immunoassay.

In some embodiments, the measurement is a molecular marker measurement. In some embodiments, the molecular marker measurement is a histone trimethylation (H3K9me3) measurement. In some embodiments, the molecular marker measurement is a protein measurement. In some embodiments, the molecular marker measurement is a brain-derived neurotrophic factor (BDNF) measurement. In some embodiments, the molecular marker measurement is an Aβ measurement. In some embodiments, the measurement includes a beta-amyloid deposit measurement. In some embodiments, the protein measurement is a measurement of a protein in FIG. 19A. The measurement of the protein in FIG. 19A may be increased, relative to the baseline measurement. The measurement of the protein in FIG. 19A may be decreased, relative to the baseline measurement. In some embodiments, the protein measurement is a measurement of a protein in FIG. 19B. The measurement of the protein in FIG. 19B may be increased, relative to the baseline measurement. The measurement of the protein in FIG. 19B may be decreased, relative to the baseline measurement. In some embodiments, the measurement is a measurement of an aspect or protein in FIG. 20. The measurement of the protein in FIG. 20 may be increased, relative to the baseline measurement. The measurement of the protein in FIG. 20 may be decreased, relative to the baseline measurement. In some embodiments, the molecular marker measurement is determined in a biopsy. In some embodiments, the molecular marker measurement is determined using an immunoassay such as an ELISA. In some embodiments, the molecular marker measurement is increased. For example, the BDNF measurement may be increased following treatment with the compound. In some embodiments, the molecular marker measurement is decreased. For example, the H3K9me3 measurement or the Aβ measurement may be decreased following treatment with the compound.

In some embodiments, the administration improves a symptom of the Alzheimer's disease. In some embodiments, the administration reduces a symptom of the Alzheimer's disease. In some embodiments, the administration prevents a symptom of the Alzheimer's disease. In some embodiments, the administration delays a symptom of the Alzheimer's disease. In some embodiments, the administration slows progression of a symptom of the Alzheimer's disease.

Described herein, are methods of delaying onset of Alzheimer's disease. In some embodiments, the delaying onset of Alzheimer's disease comprises a delay in onset of at least one symptom of Alzheimer's disease. In some embodiments, the delay in onset of at least on symptom is at least about 6 months, about 12 months, about 18 months, about 2 years, about 3 years, about 5 years, about 10 years, about 15 years, or about 20 years. In some embodiments, the delay in onset of at least on symptom is at least 6 months. In some embodiments, the delay in onset of at least on symptom is at least 12 months. In some embodiments, the delay in onset of at least on symptom is at least 18 months. In some embodiments, the delay in onset of at least on symptom is at least 2 years. In some embodiments, the delay in onset of at least on symptom is at least 3 years. In some embodiments, the delay in onset of at least on symptom is at least 5 years. In some embodiments, the delay in onset of at least on symptom is at least 10 years. In some embodiments, the delay in onset of at least on symptom is at least 15 years. In some embodiments, the delay in onset of at least on symptom is at least 20 years

In some embodiments, the delaying onset of Alzheimer's disease comprises a delay in onset of no more than one symptom of Alzheimer's disease. In some embodiments, the delay in onset of no more than on symptom is no more than about 6 months, about 12 months, about 18 months, about 2 years, about 3 years, about 5 years, about 10 years, about 15 years, or about 20 years. In some embodiments, the delay in onset of no more than on symptom is no more than 6 months. In some embodiments, the delay in onset of no more than on symptom is no more than 12 months. In some embodiments, the delay in onset of no more than on symptom is no more than 18 months. In some embodiments, the delay in onset of no more than on symptom is no more than 2 years. In some embodiments, the delay in onset of no more than on symptom is no more than 3 years. In some embodiments, the delay in onset of no more than on symptom is no more than 5 years. In some embodiments, the delay in onset of no more than on symptom is no more than 10 years. In some embodiments, the delay in onset of no more than on symptom is no more than 15 years. In some embodiments, the delay in onset of no more than on symptom is no more than 20 years

Described herein, are methods of delaying onset of at least one symptom of Alzheimer's disease. In some embodiments, the symptom comprises memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes. In some embodiments, the symptom includes memory loss. In some embodiments, the symptom includes difficulty concentrating. In some embodiments, the symptom includes difficulty completing familiar tasks. In some embodiments, the symptom includes confusion with time or place. In some embodiments, the symptom includes difficulty understanding visual images and spatial relationships. In some embodiments, the symptom includes language difficulties. In some embodiments, the symptom includes misplacing items. In some embodiments, the symptom includes decreased or poor judgement. In some embodiments, the symptom includes social withdrawal. In some embodiments, the symptom includes mood or personality changes.

In some embodiments, the method improves memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes in the subject. In some embodiments, the method improves memory loss. In some embodiments, the method improves difficulty concentrating. In some embodiments, the method improves difficulty completing familiar tasks. In some embodiments, the method improves confusion with time or place. In some embodiments, the method improves difficulty understanding visual images. In some embodiments, the method improves difficulty understanding spatial relationships. In some embodiments, the method improves difficulty understanding visual images and spatial relationships. In some embodiments, the method improves language difficulties. In some embodiments, the method improves misplacing items. In some embodiments, the method improves decreased or poor judgement. In some embodiments, the method improves social withdrawal. In some embodiments, the method improves mood or personality changes.

In some embodiments, the treatment results in improvement in a mental status test and/or a neuroimaging test. In some embodiments, the treatment results in improvement in a mental status test. In some embodiments, the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test. In some embodiments, the mental status test is a Mini-Mental State Exam (MMSE). In some embodiments, the mental status test is a Mini-Cog test. In some embodiments, the mental status test is a Cantab Mobile test. In some embodiments, the mental status test is a Cognigram test. In some embodiments, the mental status test is a Cognivue test. In some embodiments, the mental status test is a Cognision and Automated Neuropsychological Assessment Metrics (ANAM) test. In some embodiments, the improvement comprises an improved score relative to a score obtained prior to administration of the composition.

In some embodiments, the treatment results in improvement in a neuroimaging test. In some embodiments, the neuroimaging test is a magnetic resonance imaging (MRI) or computed tomography (CT). In some embodiments, the neuroimaging test is an MRI test. In some embodiments, the neuroimaging test is a CT test.

In some embodiments, the improvement comprises reduced beta-amyloid deposits as compared to an amount of beta-amyloid deposits measured prior to administration of the composition.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates. Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, —N,N-dibenzylethylenediamine, chloroprocaine, hydrabamine, choline, betaine, ethylenediamine, ethylenedianiline, —N-methylglucamine, glucosamine, methylglucamine, theobromine, purines, piperazine, piperidine, —N-ethylpiperidine, polyamine resins and the like.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### SEQUENCE INFORMATION

Some embodiments include one or more nucleic acid sequences in Table 1.

TABLE 1
Sequence Information

| SEQ ID NO: | Description |
|---|---|
| 1 | amyloid-beta precursor protein (APP) |
| | isoform a precursor [Homo sapiens] |
| | NCBI Reference Sequence: NP_000475.1 |
| 2 | presenilin-1 (PSEN-1) isoform I-467 [Homo sapiens] |
| | NCBI Reference Sequence: NP_000012.1 |
| 3 | presenilin-2 (PSEN-2) isoform 1 [Homo sapiens] |
| | NCBI Reference Sequence: NP_000438.2 |

### EMBODIMENTS

Some aspects include one or more of the following embodiments:

1. A method of treating, preventing or delaying onset of an Alzheimer's disease in a subject in need thereof, the method comprising administering to the subject a composition comprising ETP69 wherein the subject has (i) at least one mutation associated with familial Alzheimer's disease; or (ii) at least one genetic risk factor associated with sporadic Alzheimer's disease.

2. The method of embodiment 1, wherein the familial Alzheimer's disease comprises Alzheimer's disease type 1.

3. The method of embodiment 1, wherein the familial Alzheimer's disease comprises Alzheimer's disease type 3.

4. The method of embodiment 1, wherein the familial Alzheimer's disease comprises Alzheimer's disease type 4.

5. The method of embodiment 1, wherein the at least one mutation associated with familial Alzheimer's disease comprises a mutation in an amyloid precursor protein (APP) gene, a presenilin-1 (PSEN1) gene, or a presenilin-2 (PSEN2) gene.

6. The method of embodiment 5, wherein the mutation in the APP gene codes for a mutation in the amyloid precursor protein amino acid sequence that is expressed from the APP gene.

7. The method of embodiment 6, wherein the mutation in the amyloid precursor protein amino acid sequence comprises a mutation at valine 717 according to SEQ ID NO: 1.

8. The method of embodiment 7, wherein the valine 717 of the amyloid precursor protein is mutated to isoleucine (V717I), phenylalanine (V717F), glycine (V717G), or leucine (V717L).

9. The method of embodiment 6, wherein the mutation in the amyloid precursor protein amino acid sequence comprises mutations at positions lysine 670 and methionine 671 according to SEQ ID NO: 1.

10. The method of embodiment 9, wherein the lysine 670 and methionine 671 are mutated to asparagine and lysine, respectively (KM670/671NL).

11. The method of embodiment 6, wherein the mutation in the amyloid precursor protein amino acid sequence comprises a mutation at glutamate 693 according to SEQ ID NO: 1.

12. The method of embodiment 6, wherein the mutation in the APP gene codes for a deletion at position glutamate 693 of the amyloid precursor protein amino acid.

13. The method of embodiment 11, wherein the glutamate 693 of the of the amyloid precursor protein is mutated to glutamine (E693Q), glycine (E693G), or lysine (E693K).

14. The method of embodiment 6, wherein the mutation in the amyloid precursor protein amino acid sequence comprises a mutation at threonine 714 according to SEQ ID NO: 1.

15. The method of embodiment 14, wherein the threonine 714 of the of the amyloid precursor protein is mutated to isoleucine (T714I) or alanine (T714A).

16. The method of embodiment 6, wherein the mutation in the amyloid precursor protein amino acid sequence comprises a mutation at valine 715 according to SEQ ID NO: 1.

17. The method of embodiment 16, wherein the valine 715 of the of the amyloid precursor protein is mutated to methionine (V715M) or alanine (V715A).

18. The method of embodiment 6, wherein the mutation in the amyloid precursor protein amino acid sequence comprises a mutation at isoleucine 716 according to SEQ ID NO: 1.

19. The method of embodiment 18, wherein the isoleucine 716 of the of the amyloid precursor protein is mutated to valine (I716V) or phenylalanine (I716F).

20. The method of embodiment 5, wherein the mutation in the PSEN-1 gene codes for a mutation in the presenilin-1 amino acid sequence that is expressed from the PSEN-1 gene.

21. The method of embodiment 20, wherein the mutation in the presenilin-1 amino acid sequence comprises a mutation at methionine 146, leucine 166, isoleucine 213, arginine 278, or alanine 246 according to SEQ ID NO: 2.

22. The method of embodiment 21, wherein the methionine 146 is mutated to leucine (M146L), the leucine 166 is mutated to proline (L166P), the isoleucine 213 is mutated to threonine (I213T), the arginine 278 is mutated to isoleucine (R278I), and the alanine 246 is mutated to glutamate (A246E).

23. The method of embodiment 2, wherein the mutation in the PSEN-2 gene codes for a mutation in the presenilin-2 amino acid sequence that is expressed from the PSEN-2 gene.

24. The method of embodiment 23, wherein the mutation in the presenilin-2 amino acid sequence comprises a mutation at asparagine 141 or methionine 239 according to SEQ ID NO: 3.

25. The method of embodiment 24, wherein the asparagine 141 is mutated to isoleucine and the methionine 239 is mutated to valine.

26. The method of embodiment 1, wherein the genetic risk factor associated with sporadic Alzheimer's disease comprises the subject being a carrier of an apolipoprotein (APOE) e4 allele.

27. The method of embodiment 1, wherein the genetic risk factor associated with sporadic Alzheimer's disease comprises a mutation in a gene.

28. The method of embodiment 27, wherein the gene comprises ABCA7, AKAP9, BIN1, CASS4, CD2AP, CD33, CLU, EPHA1, FERMT2, HLA-DRB5/DRB1, INPP5D, MEF2C, MS4A6A/MS4A4E, PICALM, PLD3, ACE, PTK2B, —SORL1, TREM2, or UNC5C.

29. The method of any one of embodiments 1-28, wherein the subject is asymptomatic of Alzheimer's disease.

30. The method of any one of embodiments 1-29, wherein the subject is less than about 25, about 30, about 35, about 40, about 45, about 50, about, about 55, about 60, about 65, or about 70 years of age.

31. The method of any one of embodiments 1-30, wherein the delaying onset of Alzheimer's disease comprises a delay in onset of at least one symptom of Alzheimer's disease.

32. The method of embodiment 31, wherein the delay in onset of at least on symptom is at least about 6 months, about 12 months, about 18 months, about 2 years, about 3 years, about 5 years, about 10 years, about 15 years, or about 20 years.

33. The method of embodiments 31 or 32, wherein the symptom comprises memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes.

34. The method of any one of embodiments 1-28, wherein the method improves memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes in the subject.

35. The method of embodiment 34, wherein the treatment results in improvement in a mental status test and/or a neuroimaging test.

36. The method of embodiment 35, wherein the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test.

37. The method of embodiment 36, wherein the improvement comprises an improved score relative to a score obtained prior to administration of the composition.

38. The method of embodiment 35, wherein the neuroimaging test is a magnetic resonance imaging (MRI) or computed tomography (CT).

39. The method of embodiment 38, wherein the improvement comprises reduced beta-amyloid deposits as compared to an amount of beta-amyloid deposits measured prior to administration of the composition.

40. A method of improving cognition in a subject at risk of developing Alzheimer's disease, the method comprising administering to the subject a composition comprising ETP69 wherein the subject has (i) at least one mutation associated with familial Alzheimer's disease; or (ii) at least one genetic risk factor associated with sporadic Alzheimer's disease.

41. The method of embodiment 40, wherein the familial Alzheimer's disease comprises Alzheimer's disease type 1.

42. The method of embodiment 40, wherein the familial Alzheimer's disease comprises Alzheimer's disease type 3.

43. The method of embodiment 40, wherein the familial Alzheimer's disease comprises Alzheimer's disease type 4.

44. The method of embodiment 40, wherein the at least one mutation associated with familial Alzheimer's disease comprises a mutation in an amyloid precursor protein (APP) gene, a presenilin-1 (PSEN1) gene, or a presenilin-2 (PSEN2) gene.

45. The method of embodiment 44, wherein the mutation in the APP gene codes for a mutation in the amyloid precursor protein amino acid sequence that is expressed from the APP gene.

46. The method of embodiment 45, wherein the mutation in the amyloid precursor protein amino acid sequence comprises a mutation at valine 717 according to SEQ ID NO: 1.

47. The method of embodiment 46, wherein the valine 717 of the amyloid precursor protein is mutated to isoleucine (V717I), phenylalanine (V717F), glycine (V717G), or leucine (V717L).

48. The method of embodiment 45, wherein the mutation in the amyloid precursor protein amino acid sequence comprises mutations at positions lysine 670 and methionine 671 according to SEQ ID NO: 1.

49. The method of embodiment 45, wherein the lysine 670 and methionine 671 are mutated to asparagine and lysine, respectively (KM670/671NL).

50. The method of embodiment 45, wherein the mutation in the amyloid precursor protein amino acid sequence comprises a mutation at glutamate 693 according to SEQ ID NO: 1.

51. The method of embodiment 45, wherein the mutation in the APP gene codes for a deletion at position glutamate 693 of the amyloid precursor protein amino acid.

52. The method of embodiment 51, wherein the glutamate 693 of the of the amyloid precursor protein is mutated to glutamine (E693Q), glycine (E693G), or lysine (E693K).

53. The method of embodiment 45, wherein the mutation in the amyloid precursor protein amino acid sequence comprises a mutation at threonine 714 according to SEQ ID NO: 1.

54. The method of embodiment 53, wherein the threonine 714 of the of the amyloid precursor protein is mutated to isoleucine (T714I) or alanine (T714A).

55. The method of embodiment 45, wherein the mutation in the amyloid precursor protein amino acid sequence comprises a mutation at valine 715 according to SEQ ID NO: 1.

56. The method of embodiment 55, wherein the valine 715 of the of the amyloid precursor protein is mutated to methionine (V715M) or alanine (V715A).

57. The method of embodiment 45, wherein the mutation in the amyloid precursor protein amino acid sequence comprises a mutation at isoleucine 716 according to SEQ ID NO: 1.

58. The method of embodiment 57, wherein the isoleucine 716 of the of the amyloid precursor protein is mutated to valine (I716V) or phenylalanine (I716F).

59. The method of embodiment 44, wherein the mutation in the PSEN-1 gene codes for a mutation in the presenilin-1 amino acid sequence that is expressed from the PSEN-1 gene.

60. The method of embodiment 59, wherein the mutation in the presenilin-1 amino acid sequence comprises a mutation at methionine 146, leucine 166, isoleucine 213, arginine 278, or alanine 246 according to SEQ ID NO: 2.

61. The method of embodiment 60, wherein the methionine 146 is mutated to leucine (M146L), the leucine 166 is mutated to proline (L166P), the isoleucine 213 is mutated to threonine (I213T), the arginine 278 is mutated to isoleucine (R278I), and the alanine 246 is mutated to glutamate (A246E).

62. The method of embodiment 44, wherein the mutation in the PSEN-2 gene codes for a mutation in the presenilin-2 amino acid sequence that is expressed from the PSEN-2 gene.

63. The method of embodiment 62, wherein the mutation in the presenilin-2 amino acid sequence comprises a mutation at asparagine 141 or methionine 239 according to SEQ ID NO: 3.

64. The method of embodiment 63, wherein the asparagine 141 is mutated to isoleucine and the methionine 239 is mutated to valine.

65. The method of embodiment 40, wherein the genetic risk factor associated with sporadic Alzheimer's disease comprises the subject being a carrier of apolipoprotein (APOE) e4 allele.

66. The method of embodiment 40, wherein the genetic risk factor associated with sporadic Alzheimer's disease comprises a mutation in a gene.

67. The method of embodiment 66, wherein the gene comprises ABCA7, AKAP9, BIN1, CASS4, CD2AP, CD33, CLU, EPHA1, FERMT2, HLA-DRB5/DRB1, INPP5D, MEF2C, MS4A6A/MS4A4E, PICALM, PLD3, ACE, PTK2B, —SORL1, TREM2, or UNC5C.

68. The method of any one of embodiments 40-67, wherein the subject is asymptomatic of Alzheimer's disease.

69. The method of any one of embodiments 40-68, wherein the subject is less than about 25, about 30, about 35, about 40, about 45, about 50, about, about 55, about 60, about 65, or about 70 years of age.

70. The method of any one of embodiments 40-69, wherein the method improves memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes in the subject.

71. The method of embodiment 70, wherein the treatment results in improvement in a mental status test and/or a neuroimaging test.

72. The method of embodiment 71, wherein the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test.

73. The method of embodiment 72, wherein the improvement comprises an improved score relative to a score obtained prior to administration of the composition.

74. The method of embodiment 71, wherein the neuroimaging test is a magnetic resonance imaging (MRI) or computed tomography (CT).

75. The method of embodiment 74, wherein the improvement comprises reduced beta-amyloid deposits as compared to an amount of beta-amyloid deposits measured prior to administration of the composition.

76. A method of improving cognition in a subject in need thereof, the method comprising: (a) obtaining results of a genetic test for at least one mutation associated with familial Alzheimer's disease for the subject; and (b) administering a composition comprising ETP69 to the subject when the genetic test indicates that the subject has at least one mutation associated with familial Alzheimer's disease.

77. The method of embodiment 76, wherein the familial Alzheimer's disease comprises Alzheimer's disease type 1.

78. The method of embodiment 76, wherein the familial Alzheimer's disease comprises Alzheimer's disease type 3.

79. The method of embodiment 76, wherein the familial Alzheimer's disease comprises Alzheimer's disease type 4.

80. The method of embodiment 76, wherein the at least one mutation associated with familial Alzheimer's disease comprises a mutation in an amyloid precursor protein (APP) gene, a presenilin-1 (PSEN1) gene, or a presenilin-2 (PSEN2) gene.

81. The method of embodiment 80, wherein the mutation in the APP gene codes for a mutation in the amyloid precursor protein amino acid sequence that is expressed from the APP gene.

82. The method of embodiment 81, wherein the mutation in the amyloid precursor protein amino acid sequence comprises a mutation at valine 717 according to SEQ ID NO: 1.

83. The method of embodiment 82, wherein the valine 717 of the amyloid precursor protein is mutated to isoleucine (V717I), phenylalanine (V717F), glycine (V717G), or leucine (V717L).

84. The method of embodiment 81, wherein the mutation in the amyloid precursor protein amino acid sequence comprises mutations at positions lysine 670 and methionine 671 according to SEQ ID NO: 1.

85. The method of embodiment 84, wherein the lysine 670 and methionine 671 are mutated to asparagine and lysine, respectively (KM670/671NL).

86. The method of embodiment 81, wherein the mutation in the amyloid precursor protein amino acid sequence comprises a mutation at glutamate 693 according to SEQ ID NO: 1.

87. The method of embodiment 81, wherein the mutation in the APP gene codes for a deletion at position glutamate 693 of the amyloid precursor protein amino acid.

88. The method of embodiment 86, wherein the glutamate 693 of the of the amyloid precursor protein is mutated to glutamine (E693Q), glycine (E693G), or lysine (E693K).

89. The method of embodiment 81, wherein the mutation in the amyloid precursor protein amino acid sequence comprises a mutation at threonine 714 according to SEQ ID NO: 1.

90. The method of embodiment 89, wherein the threonine 714 of the of the amyloid precursor protein is mutated to isoleucine (T714I) or alanine (T714A).

91. The method of embodiment 81, wherein the mutation in the amyloid precursor protein amino acid sequence comprises a mutation at valine 715 according to SEQ ID NO: 1.

92. The method of embodiment 91, wherein the valine 715 of the of the amyloid precursor protein is mutated to methionine (V715M) or alanine (V715A).

93. The method of embodiment 81, wherein the mutation in the amyloid precursor protein amino acid sequence comprises a mutation at isoleucine 716 according to SEQ ID NO: 1.

94. The method of embodiment 94, wherein the isoleucine 716 of the of the amyloid precursor protein is mutated to valine (I716V) or phenylalanine (I716F).

95. The method of embodiment 80, wherein the mutation in the PSEN-1 gene codes for a mutation in the presenilin-1 amino acid sequence that is expressed from the PSEN-1 gene.

96. The method of embodiment 95, wherein the mutation in the presenilin-1 amino acid sequence comprises a mutation at methionine 146, leucine 166, isoleucine 213, arginine 278, or alanine 246 according to SEQ ID NO: 2.

97. The method of embodiment 96, wherein the methionine 146 is mutated to leucine (M146L), the leucine 166 is mutated to proline (L166P), the isoleucine 213 is mutated to threonine (I213T), the arginine 278 is mutated to isoleucine (R278I), and the alanine 246 is mutated to glutamate (A246E).

98. The method of embodiment 80, wherein the mutation in the PSEN-2 gene codes for a mutation in the presenilin-2 amino acid sequence that is expressed from the PSEN-2 gene.

99. The method of embodiment 98, wherein the mutation in the presenilin-2 amino acid sequence comprises a mutation at asparagine 141 or methionine 239 according to SEQ ID NO: 3.

100. The method of embodiment 99, wherein the asparagine 141 is mutated to isoleucine and the methionine 239 is mutated to valine.

101. A method of improving cognition in a subject in need thereof, the method comprising: (a) obtaining results of a genetic test for at least one mutation associated with sporadic Alzheimer's disease for the subject; and (b) administering a composition comprising ETP69 to the subject when the genetic test indicates that the subject has at least one mutation associated with sporadic Alzheimer's disease.

102. The method of embodiment 101, wherein the at least one mutation associated with sporadic Alzheimer's disease comprises a mutation in an apolipoprotein (APOE) e4 gene.

103. The method of embodiment 102, wherein the mutation in the APOE e4 gene codes for a mutation in the apolipoprotein e4 amino acid sequence that is expressed from the APOE e4 gene.

104. The method of embodiment 101, wherein the genetic risk factor associated with sporadic Alzheimer's disease comprises a mutation in a gene.

105. The method of embodiment 103, wherein the gene comprises ABCA7, AKAP9, BIN1, CASS4, CD2AP, CD33, CLU, EPHA1, FERMT2, HLA-DRB5/DRB1, INPP5D, MEF2C, MS4A6A/MS4A4E, PICALM, PLD3, ACE, PTK2B, —SORL1, TREM2, or UNC5C.

106. The method of any of the aforementioned embodiments, wherein the administration results in increased synaptic integrity in the subject as compared to a synaptic integrity measured prior to administration of the composition.

107. The method of any of the aforementioned embodiments, wherein the administration results in increased dendritic spine density as compared to a dendritic spine density measured prior to administration of the composition.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

The examples described here include results from 5 different cohorts that received single (S), boost (B), or repeated (R) injections of intraperitoneal DMSO or intraperitoneal ETP69 (10 mg/kg) in DMSO at 18 mo (4 cohorts), and one 14-months cohort. In addition, a 6th cohort received oral OraB solution or ETP69 (50 mg/kg) in OraB.

### Example 1: Treatment of a Mouse Model of Alzheimer's Disease

ETP69 was tested for activity in cognitive and visual rescue in preclinical models of early-onset Alzheimer's disease (ADtg mice). Overall, a full set of locomotor, cognitive and visual behavioral tests in ADtg mice was conducted following single or double injection of ETP69. The histologic and biochemical impact of ETP69 on Alzheimer's disease was also determined. The impact was observed on neuropathology relevant to Alzheimer's disease, and on integrity of synapses and neuronal structures. Data are shown herein for locomotor, cognitive and visual function in old and young wild-type mice, as well as old and young ADtg mice.

Transgenic models such ADtg mice may mimic the early-onset human carriers of Alzheimer's disease mutations among human patients. For example, ADtg mice may mimic Alzheimer's disease's effects on the brain or retina, and/or cognitive, behavioral or visual dysfunction associated with human Alzheimer's disease. The ADtg mouse is a double-transgenic murine model of early-onset Alzheimer's disease. ADtg mice may also be referred to as 2×Tg AD or APP_{SWE}/PS1_{ΔE9} mice [also called Tg(APPswe,PSEN1dE9)85Dbo]. These well-established double transgenic mouse models of AD harbor a chimeric mouse/human amyloid precursor protein, a humanized version overexpressing mutant human amyloid beta (A4) precursor protein 695—APP (Mo/HuAPP695swe), with the Swedish mutations (K670N, M671L) of Familial Alzheimer's Disease (FAD) and a mutant human presenilin 1 (PS1-ΔE9; with deletion in Exon 9), and both directed to CNS neurons, under the control of the prion (Prn) protein promoter. These mice produce and secrete the human amyloidogenic Aβ peptides at high levels. Levels of brain Aβ₁₋₄₂ are predominant over Aβ₁₋₄₀ and both are found to aggregate in cerebral plaques and in vascular deposits, dramatically after the age of 4-5 months. Amyloid-β plaques are accumulating in brain regions such as the cortex and the hippocampus. By the age of 5-6 months, ADtg mice also exhibit synaptic loss and neuroinflammation (reactive astrocytes and activated microglia surrounding these plaques), and at the age of 10 months mice start to exhibit signs of learning and memory deficits. Hence, 2×Tg AD mice recapitulate major features of Alzheimer's Disease amyloid pathology and may be a useful model of amyloid plaque formation, soluble Aβ₁₋₄₂ oligomers, vascular amyloidosis, enhanced phosphorylation of tau microtubule-associated protein, and neuroinflammation, leading to neurodegeneration.

In these experiments, designed in part to test the therapeutic potential of ETP69 in aged mice and in an Alzheimer's disease mouse model, a first aim was to test improved or preserved memory & spine formation, and increased BDNF by ETP69 in Aged WT mice. A second aim was to explore the therapeutic potential of ETP69 in preventing cognitive decline and synaptic loss in the aged ADtg mice.

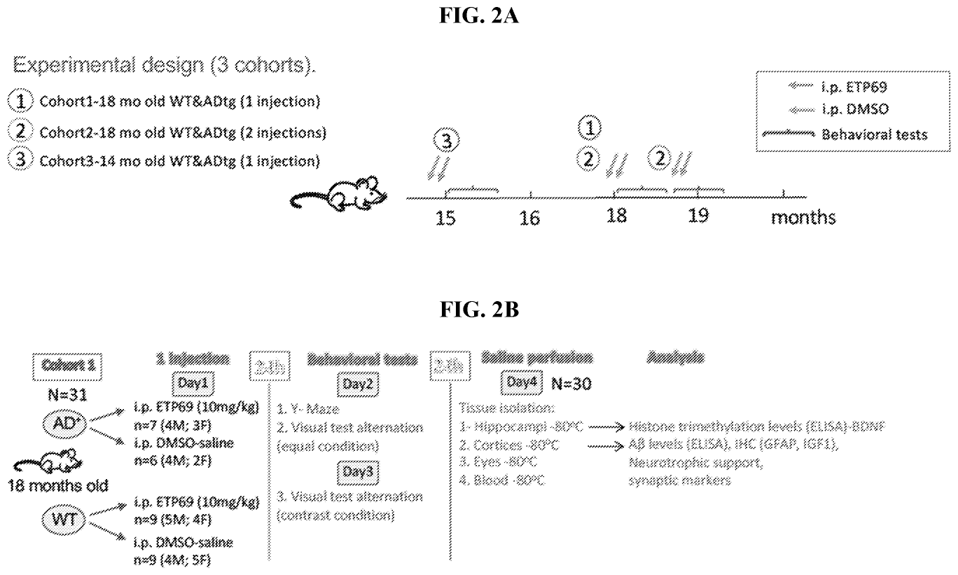

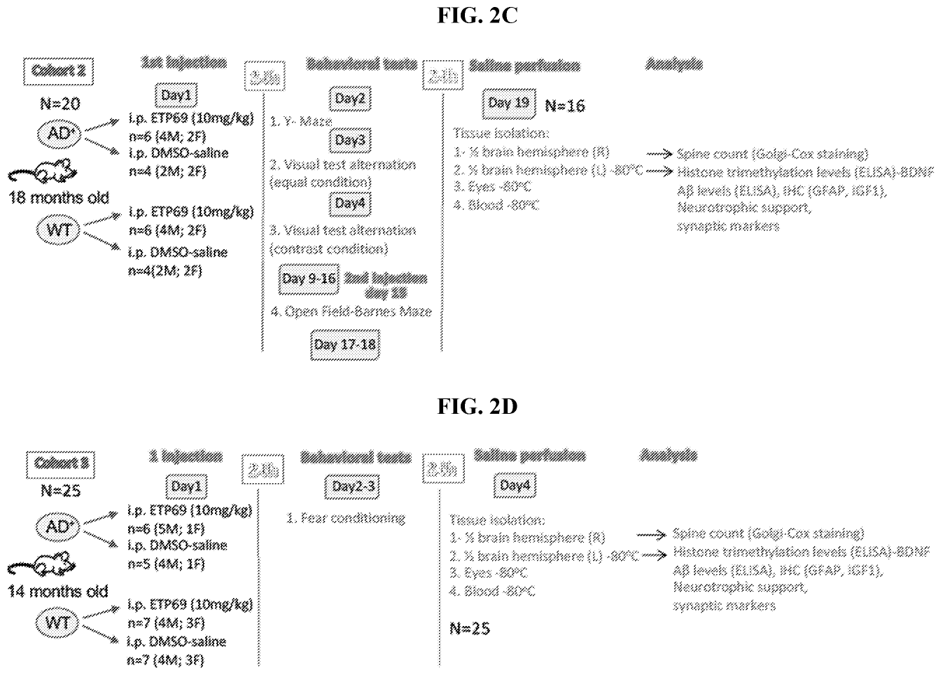

Wildtype and ADtg mice (18 months, males and females) were treated with ETP69 or vehicle (DMSO-saline) to test the effects of ETP69 in improving cognition in a mouse model of Alzheimer's disease. On day 1, mice were treated with an injection of 10 mg/kg ETP69 of vehicle. Then starting on day 2, mice were tested in various behavioral tests, including Y-maze, visual test alternation, Open Field-Barnes Maze, and/or fear conditioning. On either day 4 or day 19 mice were sacrificed, and tissues analyzed for histone trimethylation levels, A-beta levels, immunohistochemistry (GFAP, IGF1), Spine count (Golgi-Cox staining), —Neurotrophic support and synaptic markers. The experimental design is summarized in FIG. 2A-2D.

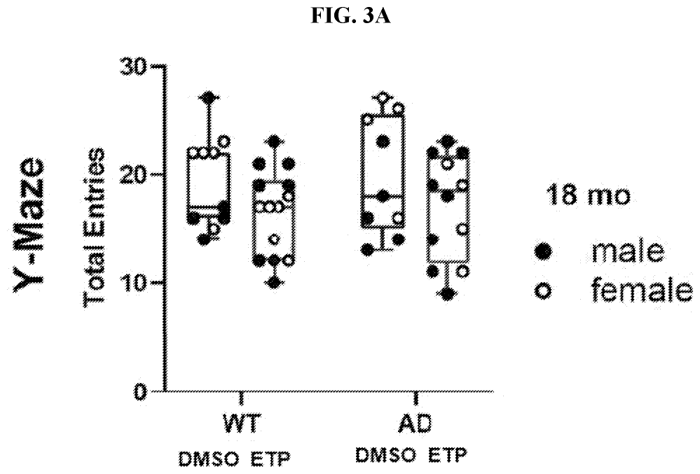

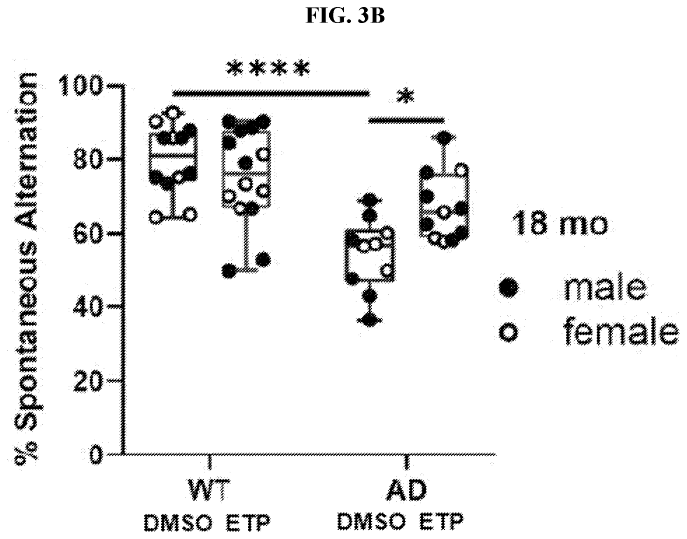

The results of the Y-maze indicate hippocampal-based spatial memory. In this test, ADtg mice treated with ETP69 showed improvement in percent spontaneous alternation compared with vehicle treated mice (FIG. 3A-3B).

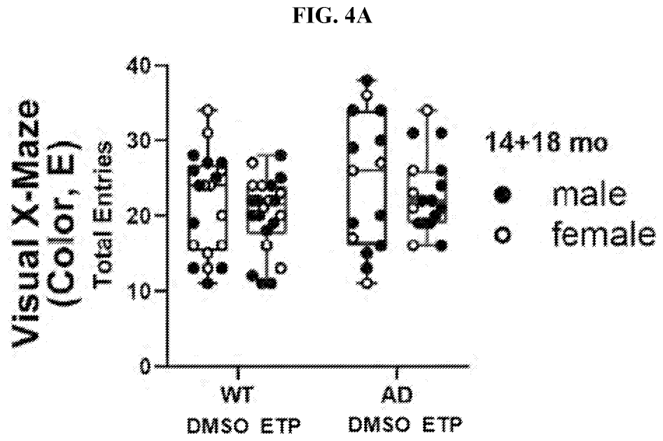

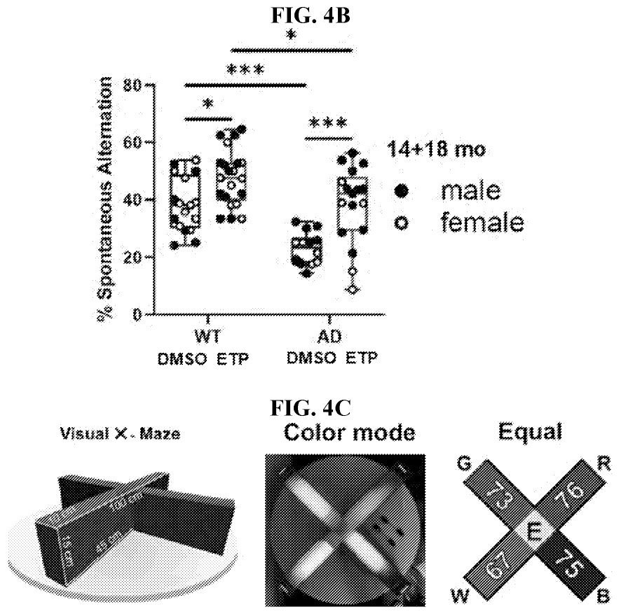

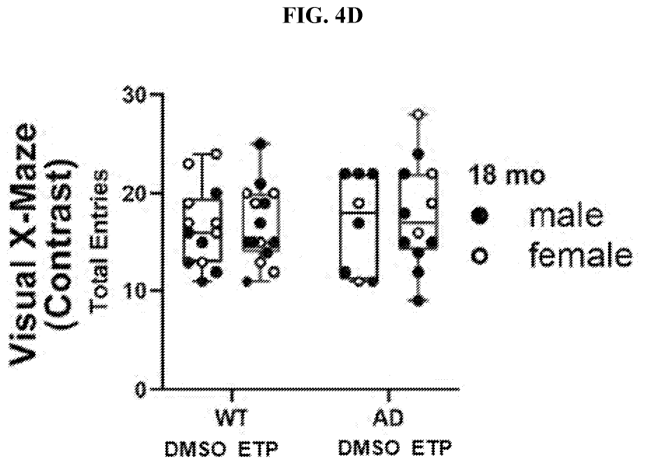

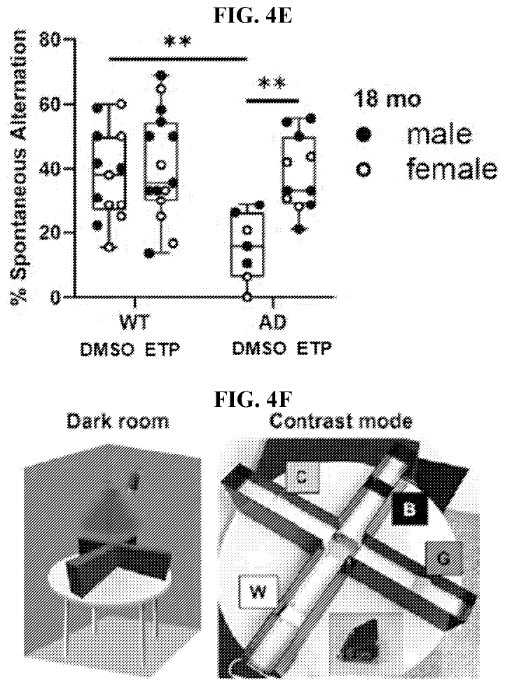

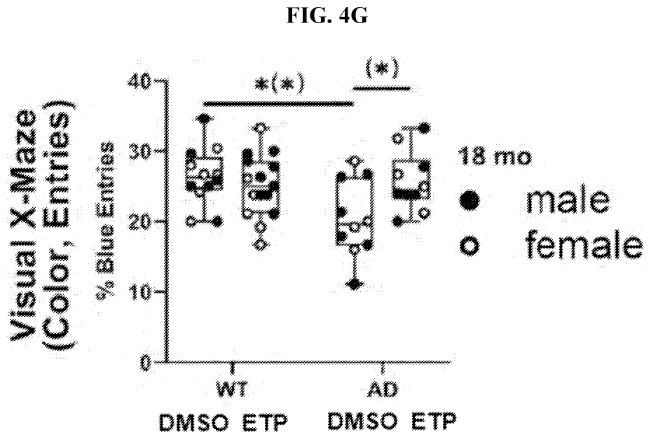

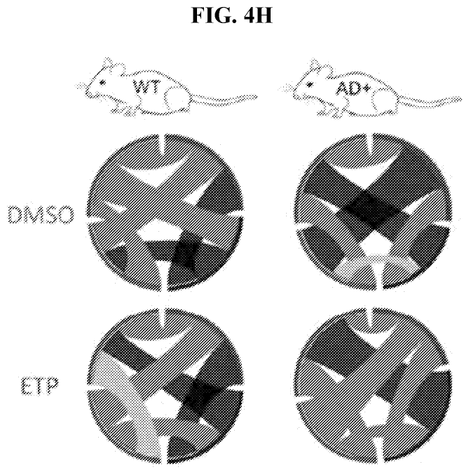

The results of the Koronyo's Visual X Maze (color) indicate visual-cognitive memory and learning. In this test ADtg mice treated with ETP69 showed improvements in percent spontaneous alternation and color bidirectional compared with vehicle treated mice (FIG. 4A-4J).

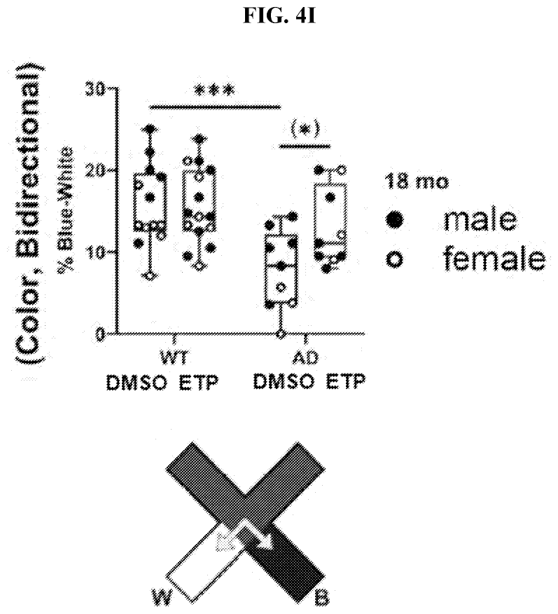

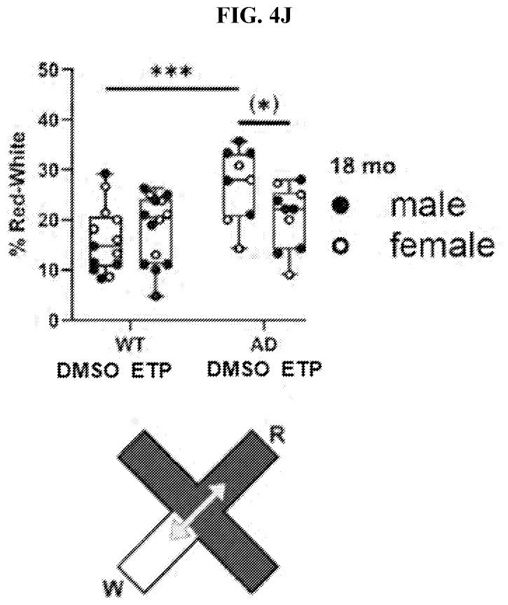

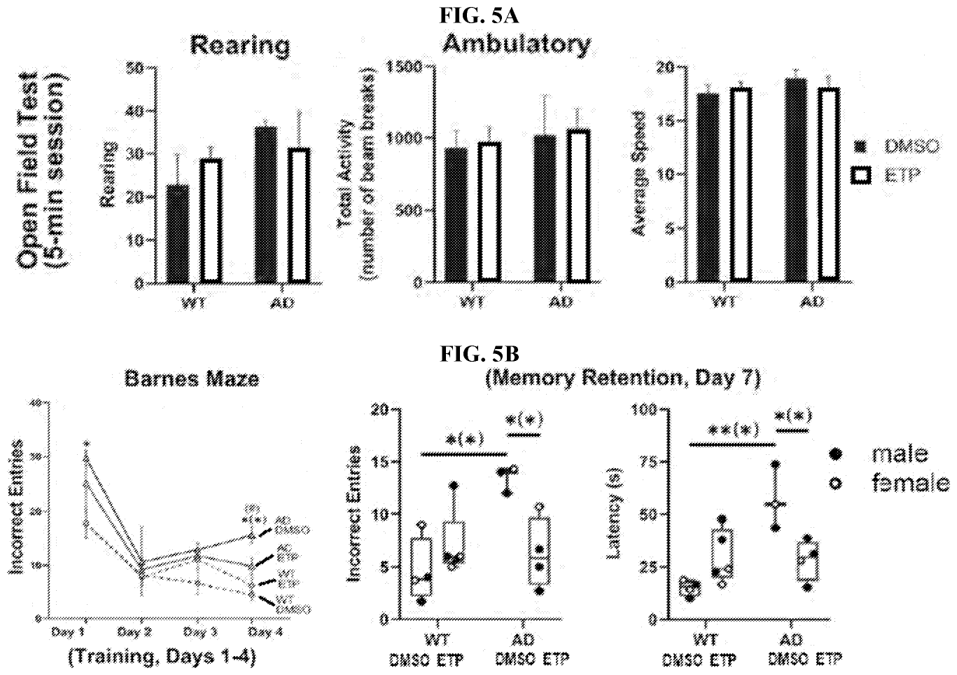

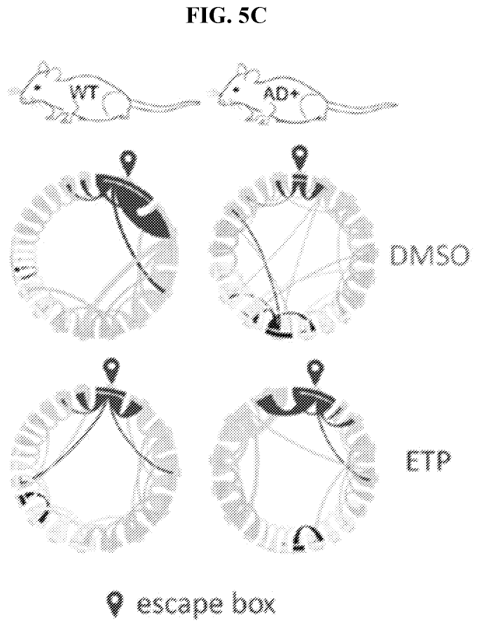

The results of the Open Field-Barnes Maze test indicate hippocampal-based spatial learning and memory. In this test, ADtg mice treated with ETP69 showed improvement in memory retention with fewer incorrect entries and reduced latency compared with vehicle treated mice (FIG. 5A-5C).

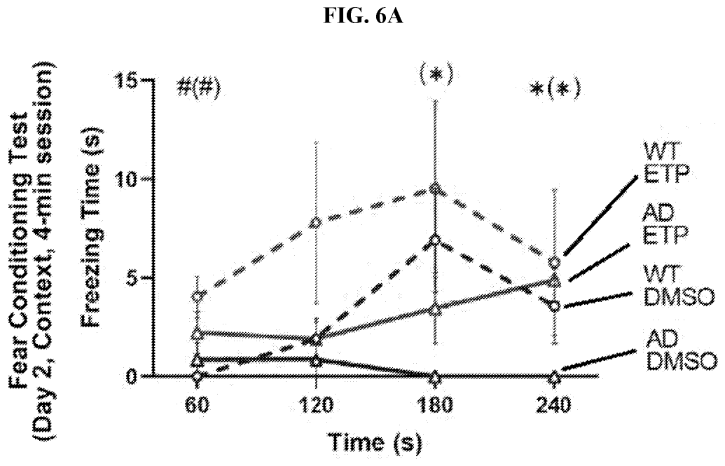

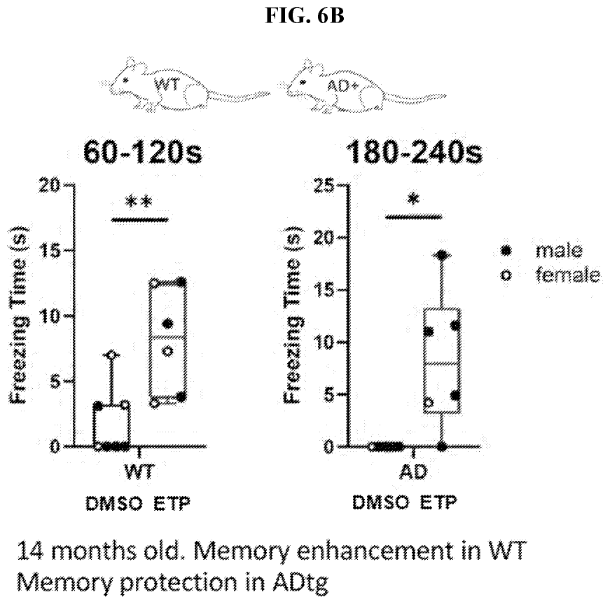

The results of the fear conditioning test indicate spatial memory. In this test, ADtg mice treated with ETP69 showed memory protection with increased freezing time (FIG. 6A-6B).

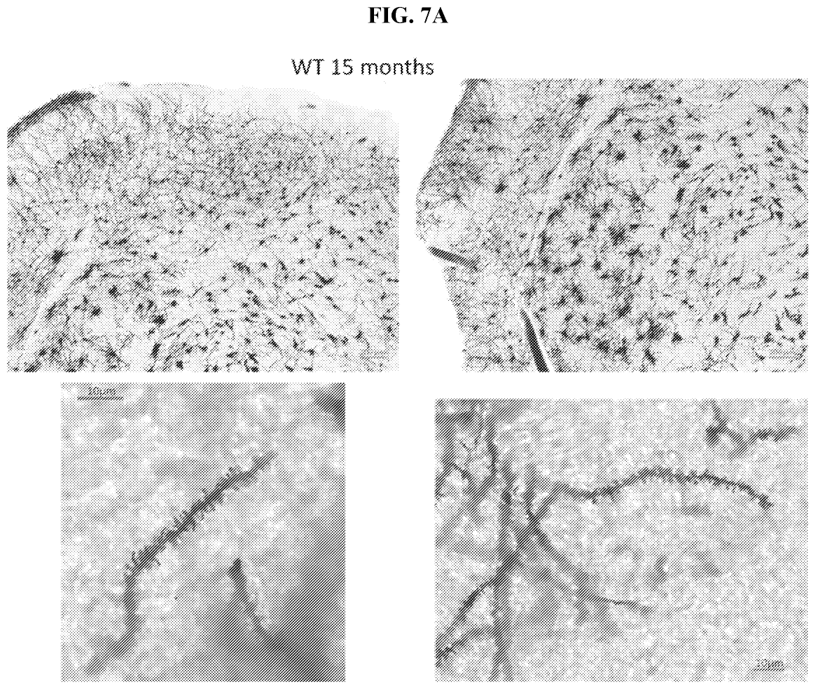

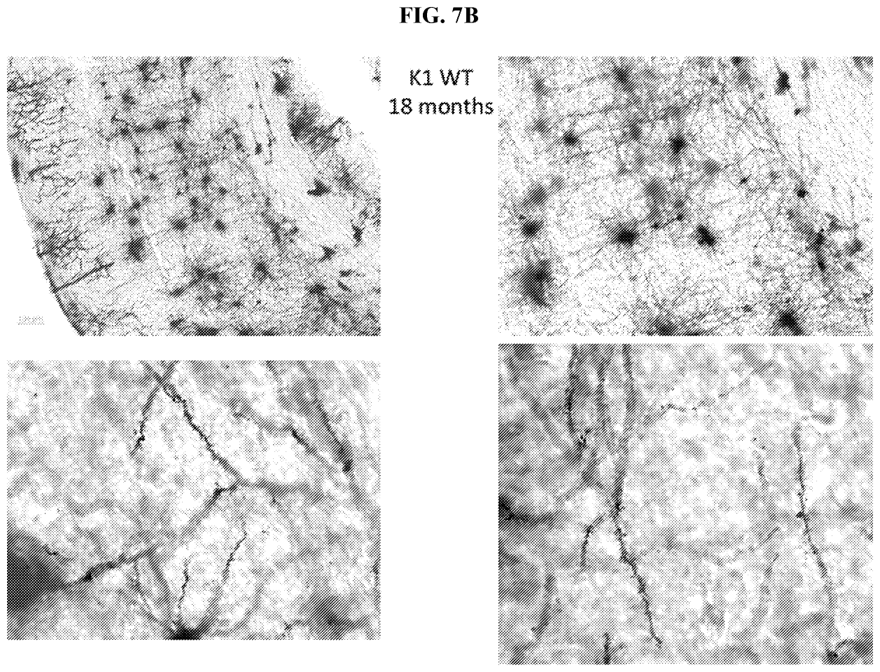

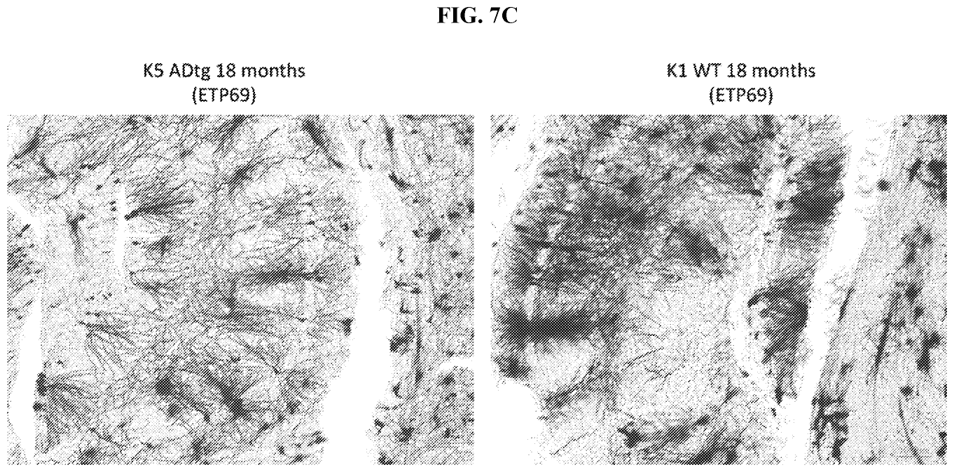

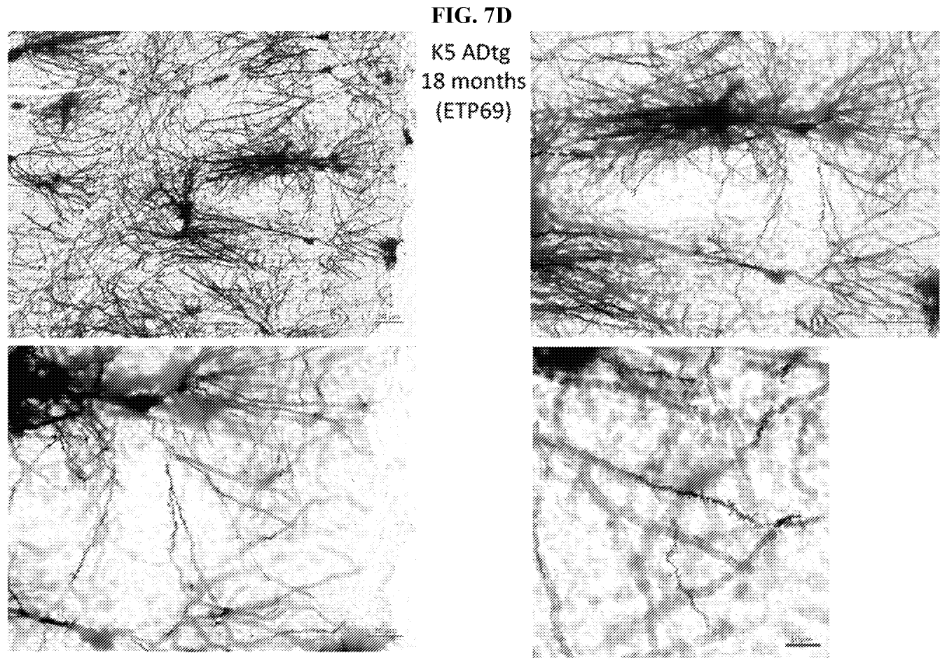

Golgi-cox staining of brain histology sections shows integrity of neuronal spines and neuronal architecture. The Golgi-Cox method is a reliable, widely used method to study cytoarchitecture of the brain, demonstrating neuronal morphology and dendritic arborization with a low background. Chromium salts which bind to proteins in the neuron are randomly formed during the impregnation, then transformed to black mercuric sulfide deposits upon alkali treatment (Ramón-Moliner, 1970; Špaček, 1989; Rosoklija et al., 2014). The architecture of the impregnated neuron, including cell somas, axons, dendrites and spines, could be easily visualized. It is expected that qualitative observations by these measures of brains from ADtg mice treated with ETP69 will show higher spine density as compared to brains of DMSO-injected control ADtg mice. FIG. 7A-7D include Golgi-Cox stain images of whole neurons and their dendritic projections.

A point of novelty of this study is exploring the therapeutic effects of ETP69 in ADtg mice as well as old wild-type mice. This makes the results of these studies relevant to early-onset Alzheimer's disease, whereas earlier studies may not have been informative of this. A goal of these studies was to test potential visual and cognitive preservation and protection by ETP69, using ADtg mice. The set of behavioral data indicates significant protective effects. Using ETP69, a specific inhibitor of SUV39H1, further studies will assess additional parameters related to Alzheimer's disease associated pathology, neurotrophic secretion, histone methylation and synaptic/neuronal integrity. Overall, these experiments provide evidence that ETP69 may preserve neuronal network integrity and cognitive and visual function. Cognitive and visual preservation in response to treatment with ETP69 is relevant to neuroscience and neuro-ophthalmology fields (e.g., neurology, neurosurgery, and ophthalmology), and medical practitioners in these fields may benefit by use of this data.

Additional experiments will analyze synapse integrity and brain cell structure following administration of ETP69 to murine models of familial Alzheimer's disease. Histological and biochemical methods will be used to assess amyloid-beta related Alzheimer's disease-relevant pathology, to further confirm molecular mechanisms, and further assess synaptic and neuronal preservation and regeneration.

These results are applicable to methods of administration of histone methyl transferase inhibitors to treat and rescue cognitive and/or visual dysfunctions in early-onset, late-onset, familial forms, or other forms of Alzheimer's disease.

### Example 2: Alzheimer's Disease Treatment

The effects of single and repeated ETP69 injections in mice were tested. The experimental timeline is depicted in FIG. 8. Three parallel injection conditions were tested: a single ETP69 condition, a boosted ETP69 injection, and repeated ETP69 injections, as depicted in Table 2. The single injection mice received one injection of ETP69 or DMSO at Day 0. The mice that received a boosted injection received one injection of ETP69 or DMSO at day 0 and one injection at day 9. The mice that received repeated injections received 11 injections of ETP69 or DMSO at day 0 or day 9. Both wildtype and AD model (APP/PS1-transgenic) mice were tested.

TABLE 2
Treatment groups

| | WT- | WT- | | | |
|---|---|---|---|---|---|
| | DMSO | ETP69 | AD+-DMSO | AD+-DMSO | Total |
| Single ETP69 | 21 | 22 | 15 | 18 | 76 |
| Boost EPT69 | 4 | 6 | 3 | 5 | 18 |
| Repeated ETP69 | 7 | 6 | 7 | 6 | 26 |

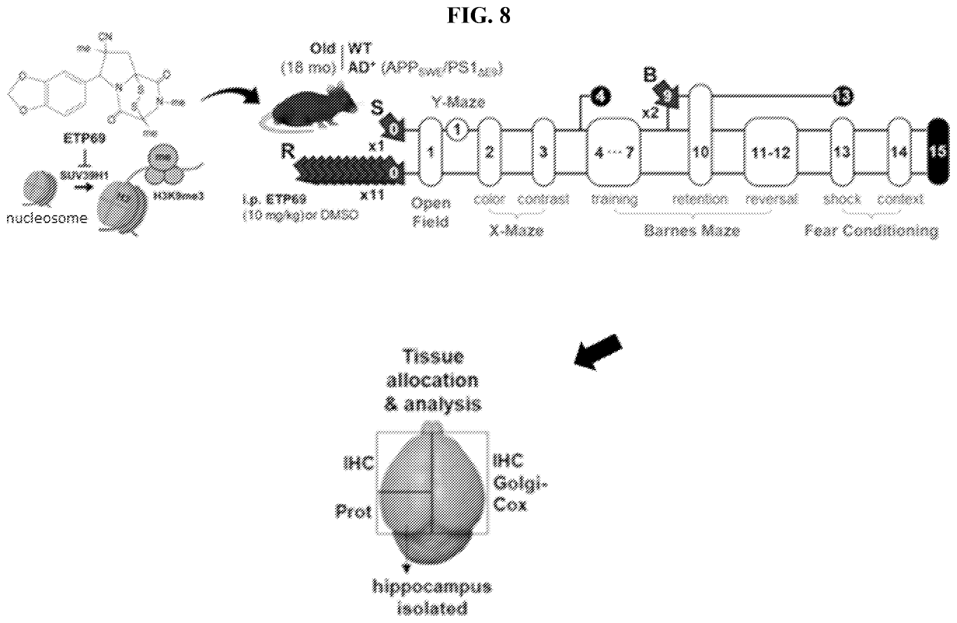

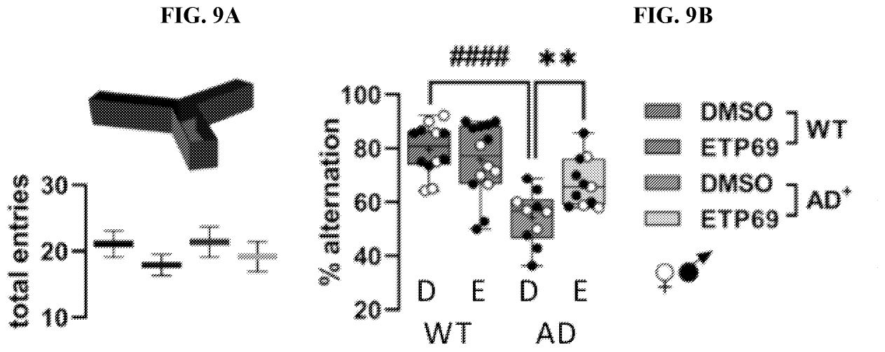

The effects of ETP69 on cognitive and visual protection was tested using the Y-Maze test. Both wildtype and AD+ mice treated with ETP69 showed a decreased number of total entries (FIG. 9A), indicating decreased locomotor activity. When looking at the percent alternations, a proxy for cognition, AD+ mice administered DMSO showed a significant decrease compared to wildtype mice administered DMSO. However, when AD+ mice were treated with ETP69, there was a significant increase in the percent alternation (FIG. 9B).

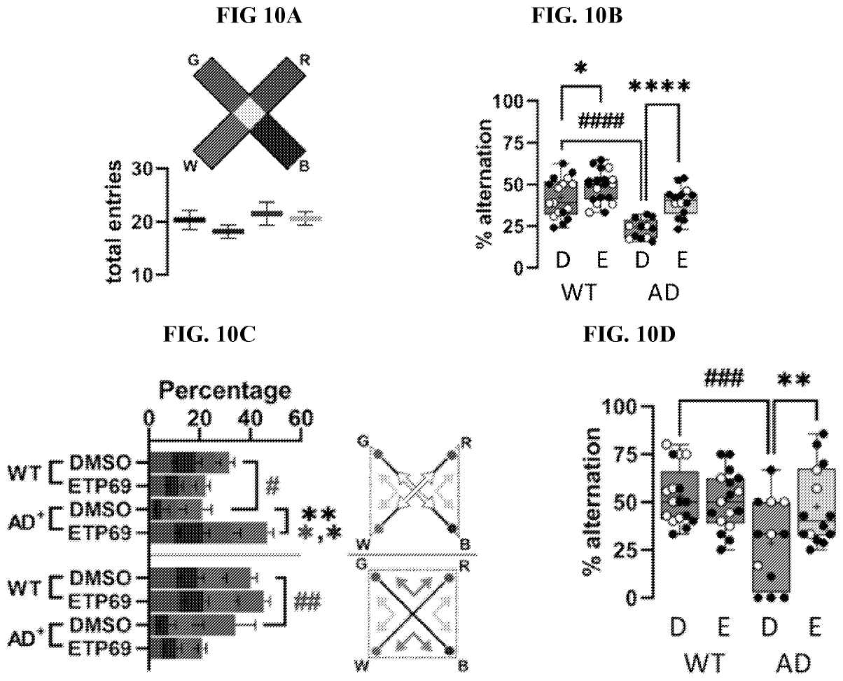

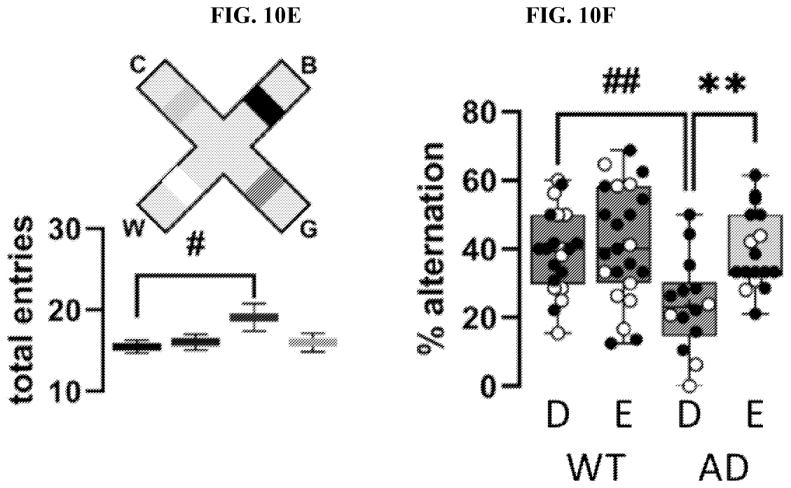

FIG. 10A-10D depicts the results of the mice on the color mode of the visual stimuli X-maze test. When looking at the percent alternations, a proxy for cognition, AD+ mice administered DMSO showed a significant decrease compared to wildtype mice administered DMSO. However, when AD+ mice were treated with ETP69, there was a significant increase in the percent alternation and bidirectional movement (FIG. 10A-10D). FIG. 10E-10F depict the results of the contrast mode of the visual stimuli X-maze test. When looking at the percent alternations, AD+ mice administered DMSO showed a significant decrease compared to wildtype mice administered DMSO. However, when AD+ mice were treated with ETP69, there was a significant increase in the percent alternation.

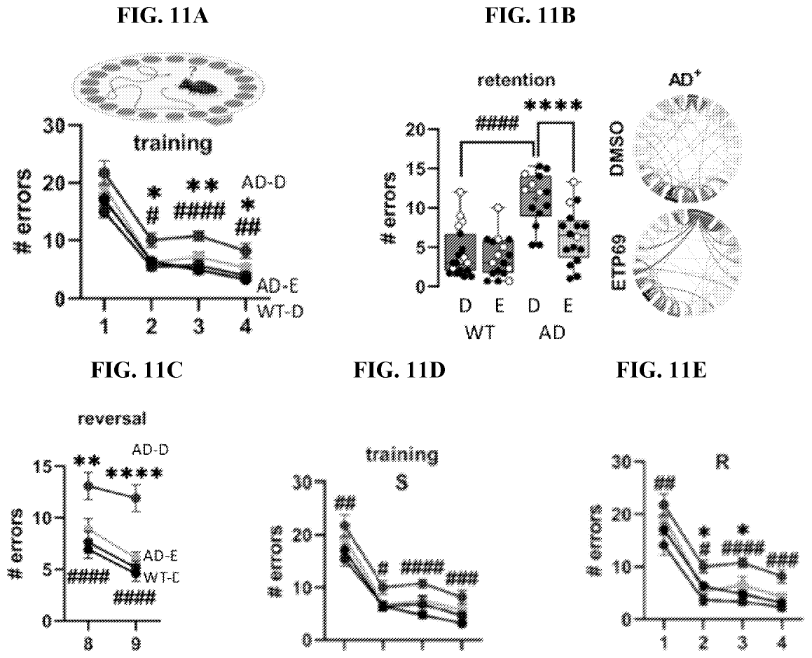

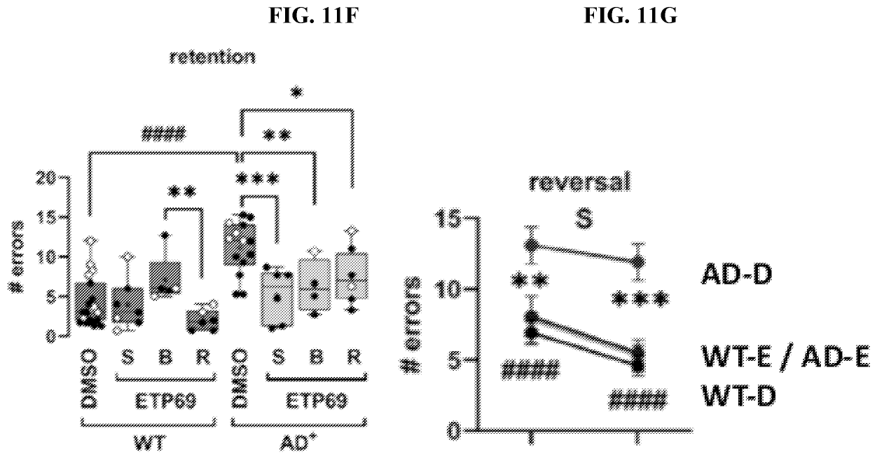

The results of the Open Field Barnes Maze test indicate hippocampal-based spatial memory and learned. AD+ mice treated with ETP69 showed an improvement in memory retention compared to AD+ mice treated with DMSO (FIG. 11A-11C). A comparison of results of single (S), boost (B) and repeated (R) injections is depicted in FIG. 11D-11H.

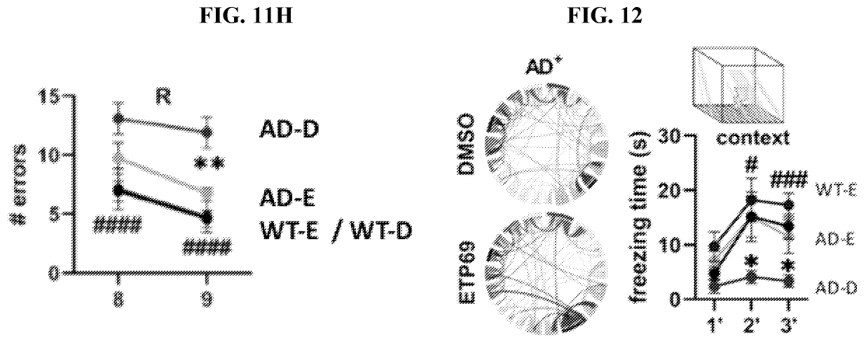

The results of the fear conditioning test indicate spatial memory. AD+ mice treated with ETP69 showed increased freezing time, indicating memory protection (FIG. 12).

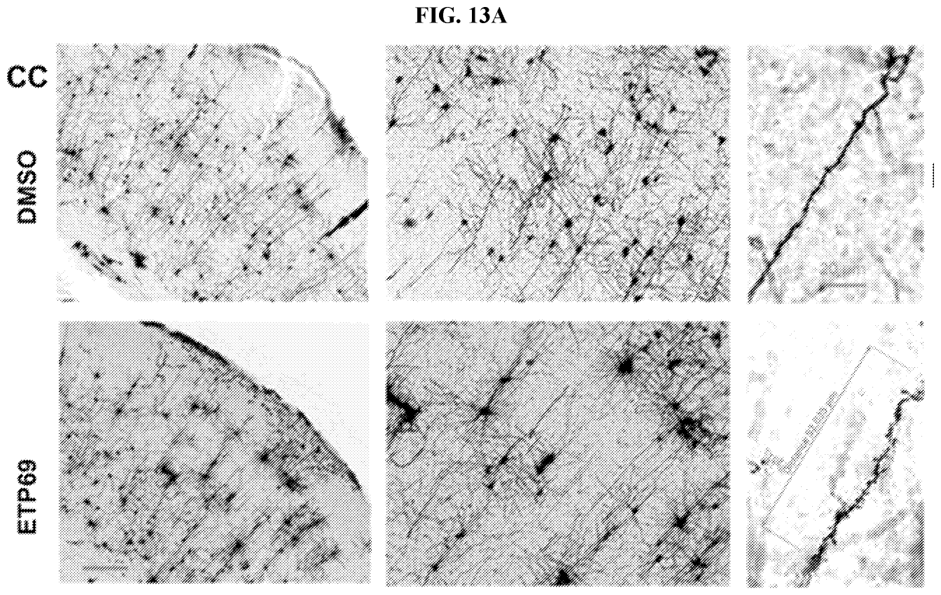

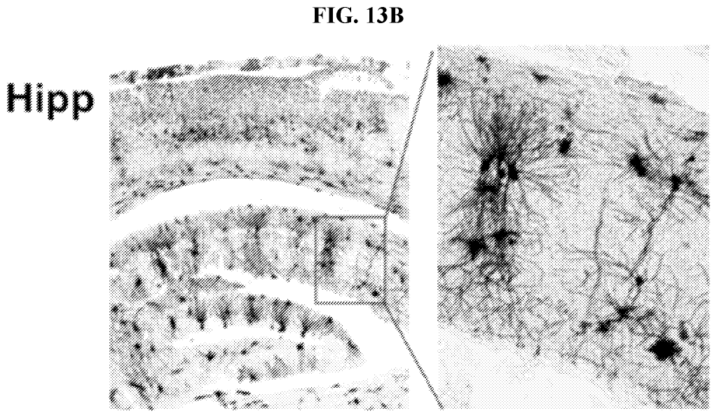

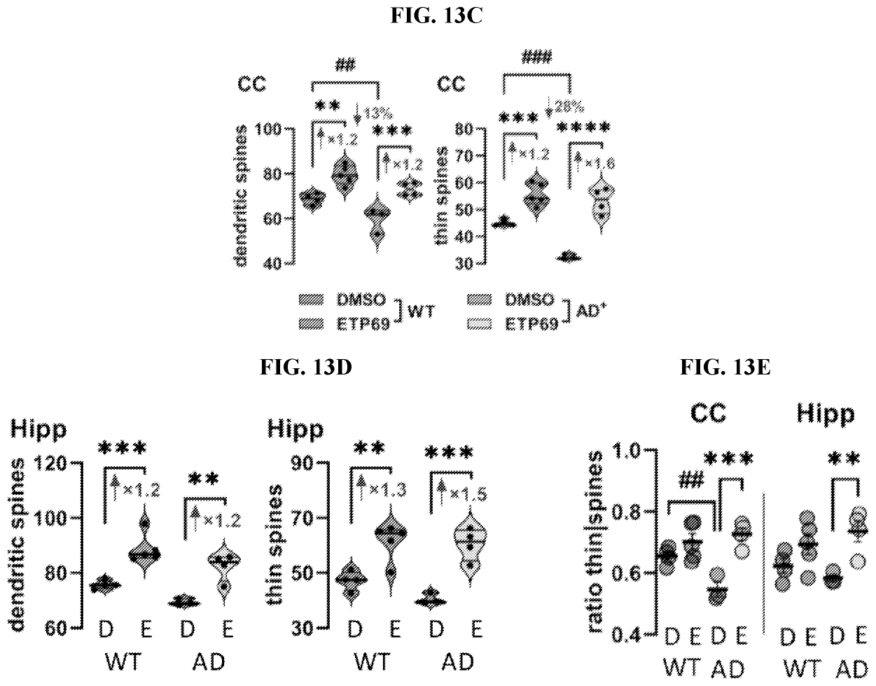

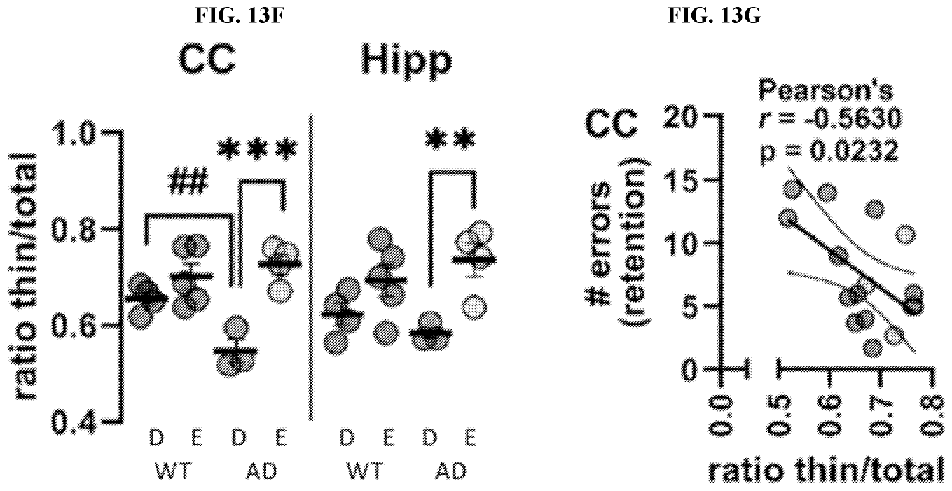

The right brain hemispheres of 18 month old mice were stained with the Golgi-Cox method to detect the cytoarchitecture and morphology of neurons. FIG. 13A depicts representative images of the cingulate cortex of mice administered DMSO or ETP69 and FIG. 13B depicts a representative image of the hippocampal area of a mouse treated with ETP69. Quantification of the dendritic spines and the thin spines in the cingulate cortex and hippocampus shows an increase in both dendritic spines and thin (immature) spines in mice treated with ETP69 (FIG. 13C-13D). Further, AD+ mice treated with ETP69 showed a significant increase in the ratio of thin spines to all spines, compared to AD+ mice administered DMSO alone (FIG. 13E-13F). Spine preservation by ETP69 was associated with reduced errors in the Barnes maze test (FIG. 13G).

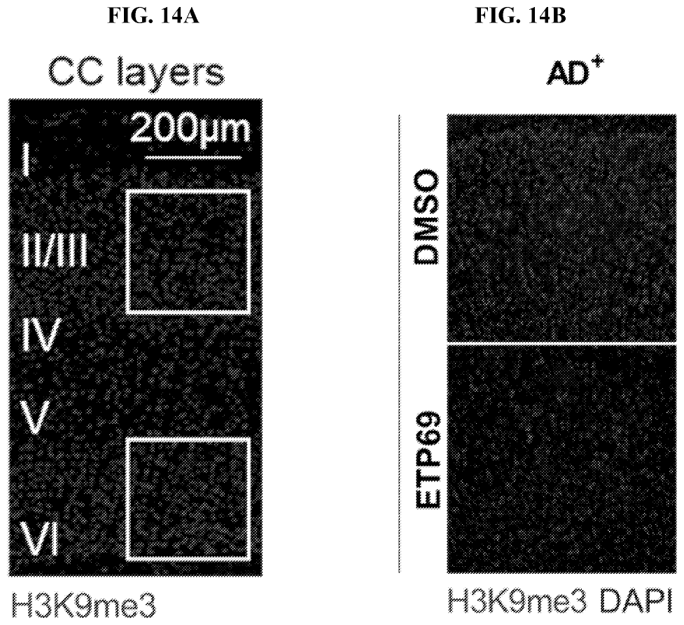

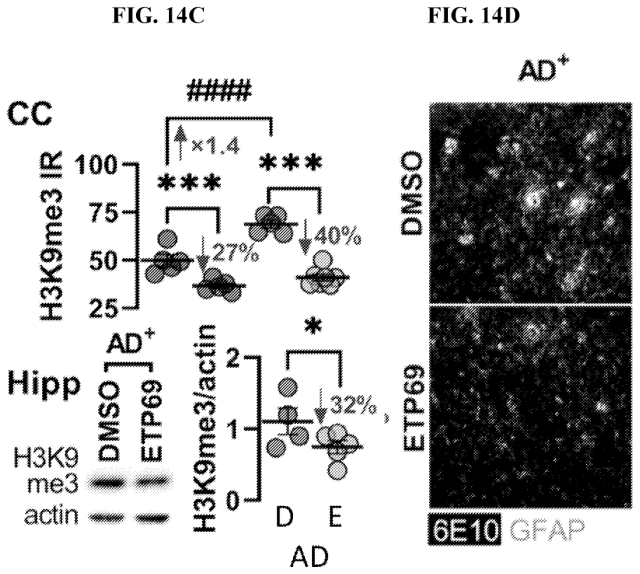

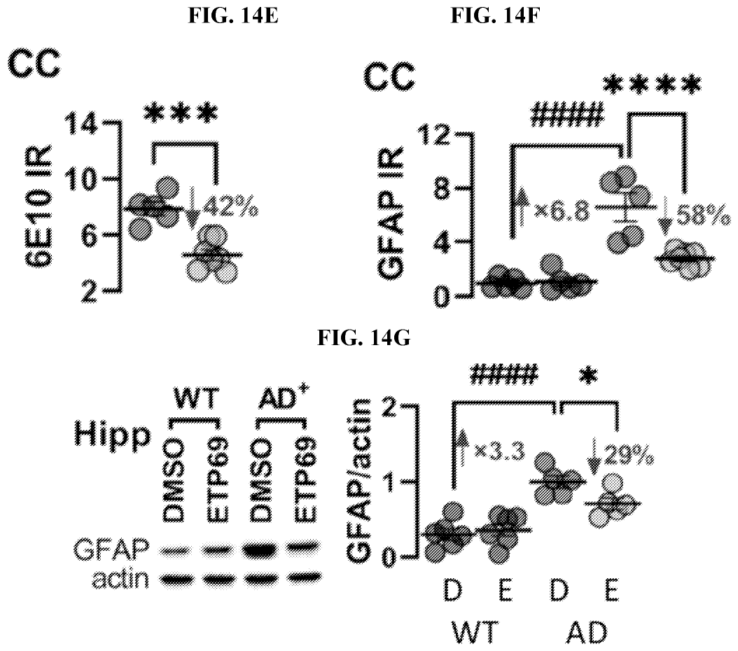

18 month old AD+ mice brains were stained for neuronal H3K9me3 and DAPI. FIG. 14A is a representative image of the H3K9me3 signal across cortical layers. FIG. 14B is a representative image comparing ETP69 treated and DMSO control brains. Quantification of H2K9me3 staining showed a significant decrease in TEP69 treated mice compared to wildtype mice in both the cortex and the hippocampus (FIG. 14C). FIG. 14D depicts immunohistochemistry of 6E10+ amyloid-beta plaques and GFAP+ astrogliosis in coronal sections of AD+ mice treated with ETP69 or DMSO. Quantification shows a 42% decrease in the amount of 6E10 amyloid plaques detected in the brains of mice treated with ETP69 (FIG. 14E). AD+ mice showed a 6.8 times increase in GFAP+ astrogliosis compared to wildtype mice. Treating AD+ mice with ETP69 resulted in a 58% decrease of GFAP irradiance (FIG. 14F). Quantitative western blot and immunohistochemical analysis showed similar effects on GFAP levels in the hippocampus (FIG. 14G).

These results are applicable to methods of treating Alzheimer's disease by administering a histone methyl transferase inhibitor such as ETP69 to a subject, and provide evidence that the compositions disclosed herein are useful for treating cognitive and biochemical defects associated with Alzheimer's disease, including familial Alzheimer's disease, early-onset Alzheimer's disease, and middle to late stage Alzheimer's disease.

### Example 3: Neuroprotective Effects of ETP69

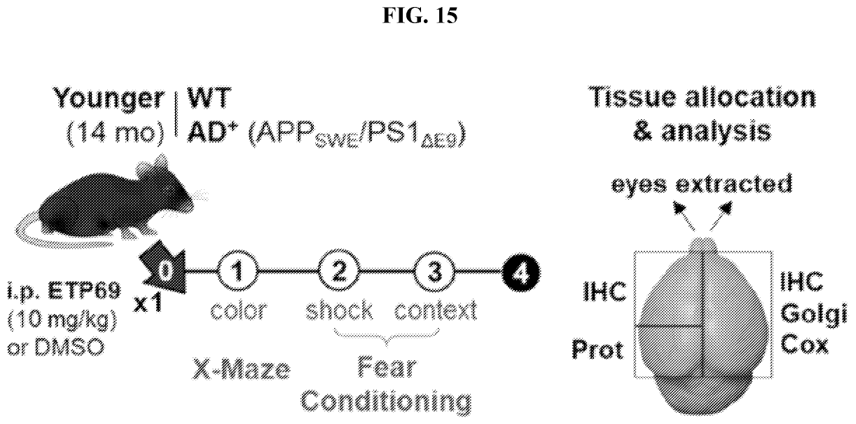

The effects of ETP69 injections in 14 month old mice was tested. Wildtype mice were divided into 2 treatment groups, mice that received an ETP69 injection (n=10) and mice that received a DMSO injection (N=10). AD+ mice were divided into 2 treatment groups, mice that received an ETP69 injection (n=8) and mice that received a DMSO injection (N=7). The experimental protocol is depicted in FIG. 15.

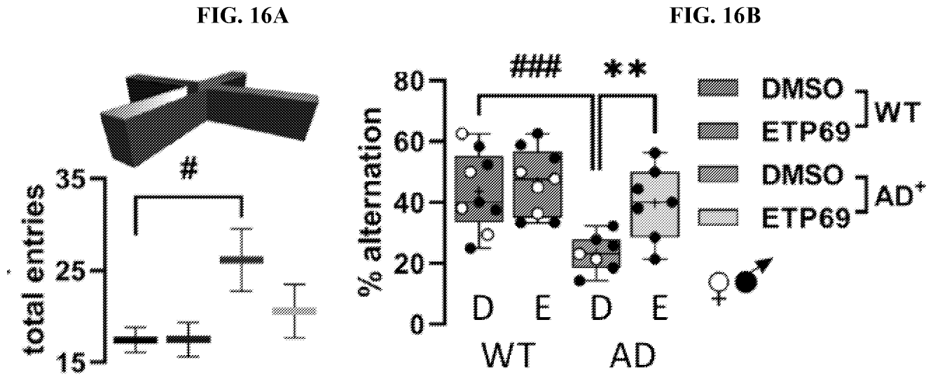

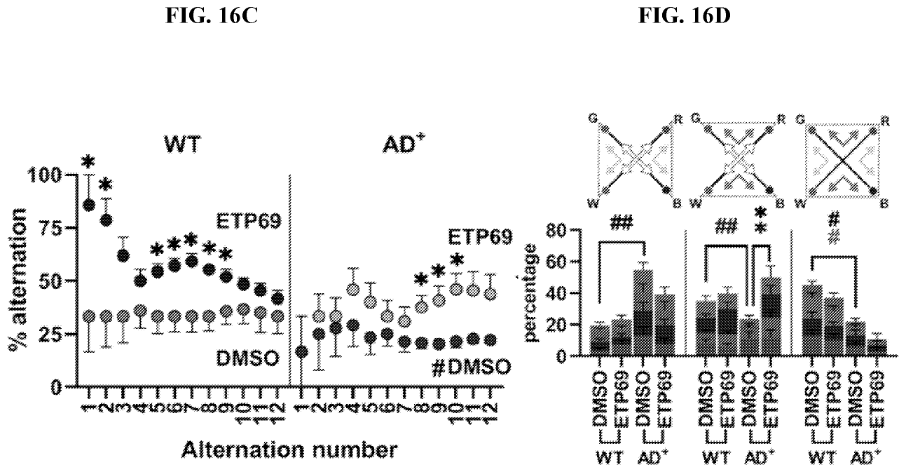

Mice were tested for visual-cognitive memory and learning using a visual X maze. ADP+ mice treated with ETP69 showed improvements in alternation compared to untreated mice (FIG. 16A-16D).

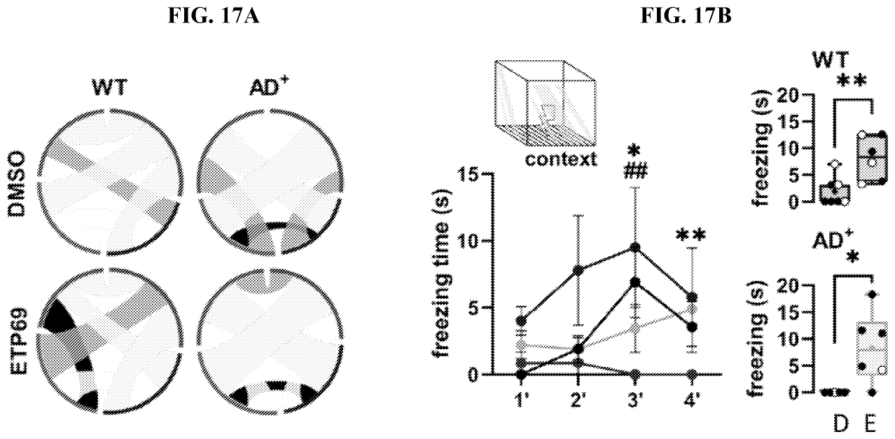

The fear conditioning test was used to indicate spatial memory. AD+ mice treated with ETP69 showed an increase in freezing time, indicating memory protection (FIG. 17A-17B).

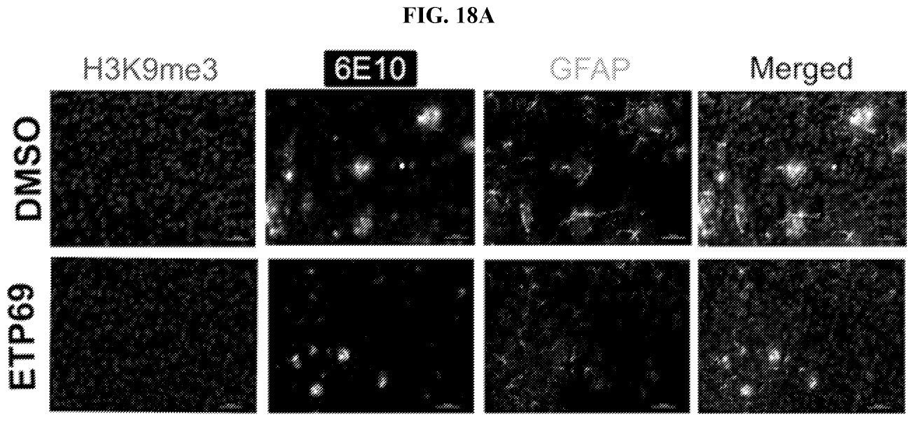

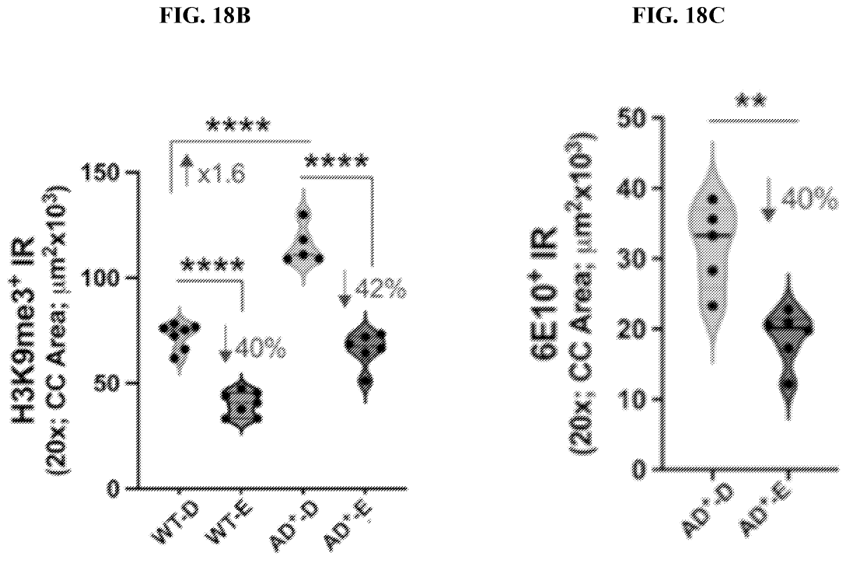

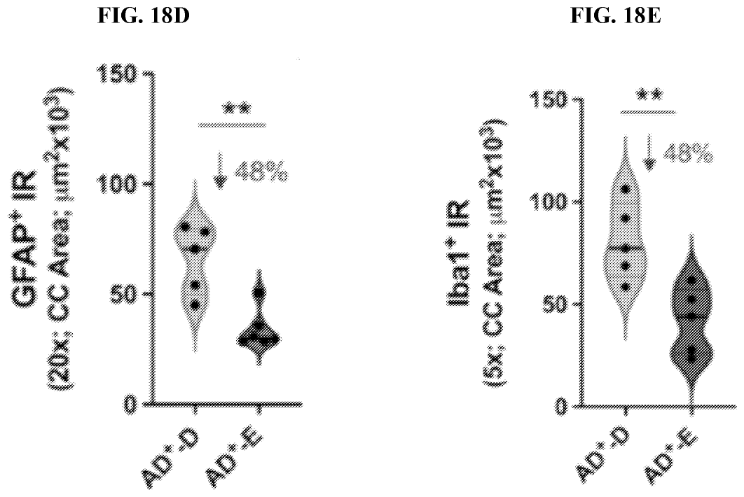

Brain sections were stained for H3K9me3, 6E10, and GFAP, Representative images are shown in FIG. 18A. Quantification of the immunohistochemistry shows that AD+ mice treated with ET69 show a decrease in H3K9me3, 6E10, GFAP and Iba1 compared to untreated AD+ mice (FIG. 18B-18E).

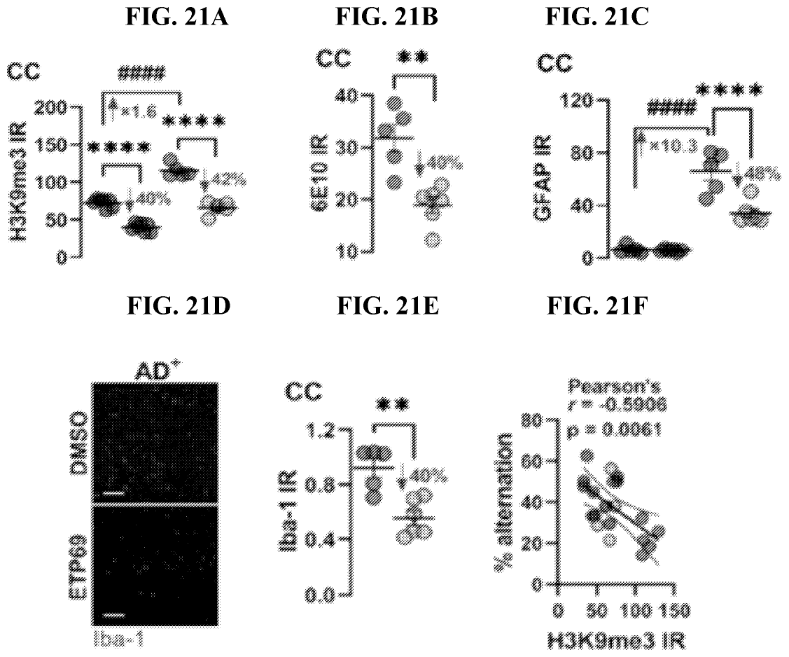

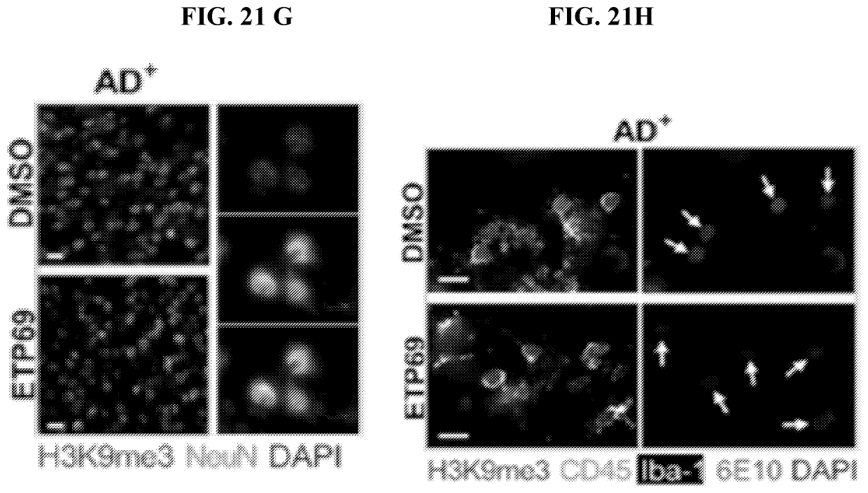

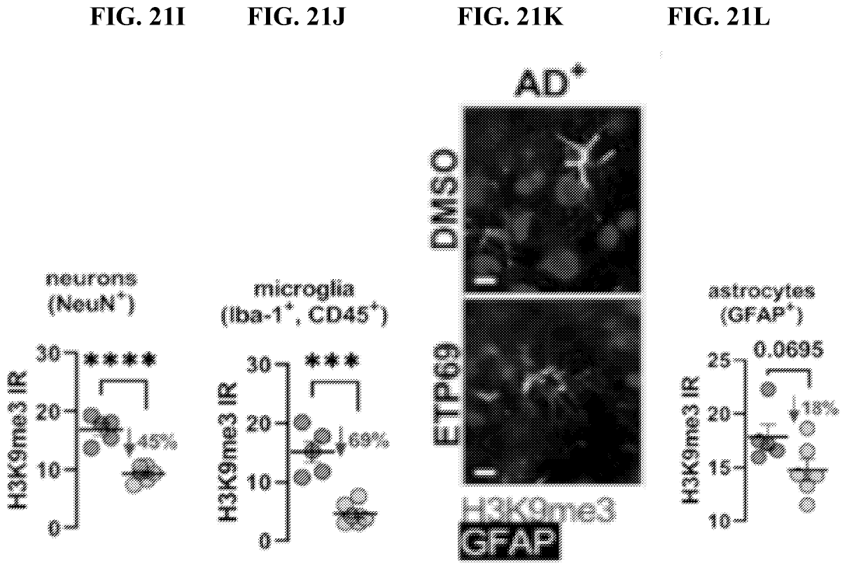

Extending beyond neurons, further experiments indicated dramatic effects of ETP69 on cerebral macrophages, microglia, and astrocytes (FIG. 21A-21L). These phagocytic inflammatory cells may be involved in immune activation and clearing toxic Aβ. Restoring a young phenotype may explain reduced neuroinflammation and Aβ plaques in the ETP69 treated AD+ mice.

These results are applicable to methods of preventing or delaying onset of Alzheimer's disease by administering a histone methyl transferase inhibitor such as ETP69 to a subject, and provide evidence that the compositions disclosed herein are useful for preventing or delaying onset of cognitive and biochemical defects associated with Alzheimer's disease, including familial Alzheimer's disease, early-onset Alzheimer's disease, and middle to late stage Alzheimer's disease.

Overall, the experiments in these examples from multiple cohorts of 14- and 18-month-old murine models of AD (AD+ mice) and age/gender-matched control mice provide support that by inhibiting the enzyme SUV39H1 that regulates H3K9me3 via ETP69 compound (AKA NT1721), the chromatin state of subjects may be altered, and memory and synaptic plasticity may be restored in aging and AD brains. The therapeutic usefulness of ETP69 has been shown in the experiments described in these examples, in both aged WT mice and the well-established transgenic mouse models of AD (APPSWE/PS1ΔE9, AD+ mice), following a single (S), a boost (B), or once a week repeated (R) i.p. injections of ETP69. The data indicate that significant reductions were achieved in cerebral H3K9m3 along with reduced AD associated pathology (amyloidosis and neuroinflammation), restoration of synaptic integrity as spine density, and preservation of various aspects of cognitive functions. Additionally, in investigation of whole proteome profiles by mass spectrometry identified some mechanisms of action for ETP69 in the aged WT and AD+ mice. Among the most up-regulated proteins following ETP69 administration were VGF and HP/HPX, which may be involved in BDNF/TrkB signaling and anti-oxidative-stress/anti-inflammatory pathways, respectively. Experiments with ETP69 have shown it to be generally safe and tolerable in humans. Hence, ETP69 and related compounds is useful in treating age-related neurological diseases, in reversing biological clocks, preserving neuronal network integrity, or in restoring cognitive or visual functions.

### Example 4: Mass Spectrometry Analysis of Pathway Activation by ETP69

FIGS. 19A, 19B, and 20 include global proteome data from a mass spectrometry analysis. FIG. 19A depicts a comparison of proteins upregulated in AD+ mice treated with ETP69 compared to untreated AD+ mice. Proteins such as VAV3, CORO2A and HPX were upregulated in the untreated AD+ brains. Some of these proteins, such as HPX, may be involved in anti-oxidative-stress or anti-inflammatory pathways. Analysis shows that the BDNF pathway is activated by ETP69 (FIG. 19B. Z-score: 2.359, p<0.0001). An ingenuity analysis shows that ETP69 results in activation of proteins related to learning and cognition and inhibition of proteins related to conditioning, anxiety, and other behaviors (FIG. 20).

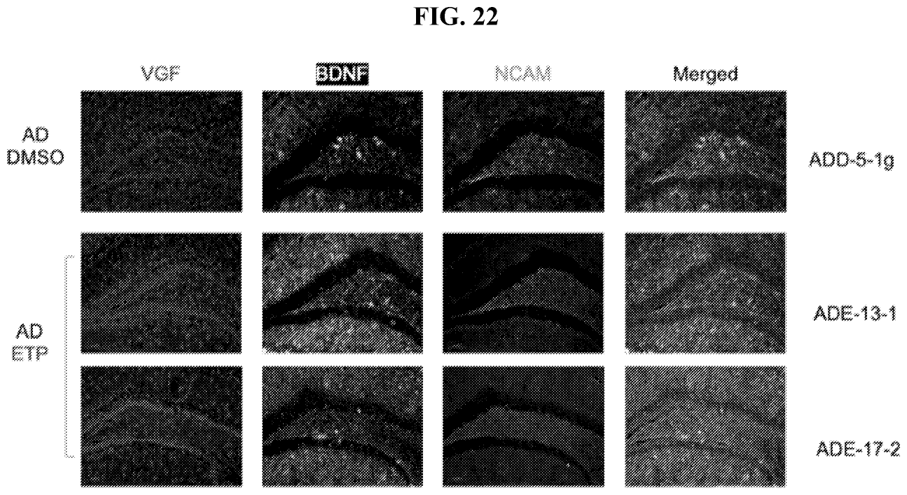

Expression of VGF, BDNF, and NCAM was analyzed in 18-month AD+ mice following a single ETP69 injection. Representative images are depicted in FIG. 22. Marked increases of BDNF and VGF expression was observed in the dentate gyrus and other hippocampal regions of 18-mo-old AD-Tg mice following ETP69 treatment.

### Example 5: Effects of Oral ETP69 in 18 Month Old Mice

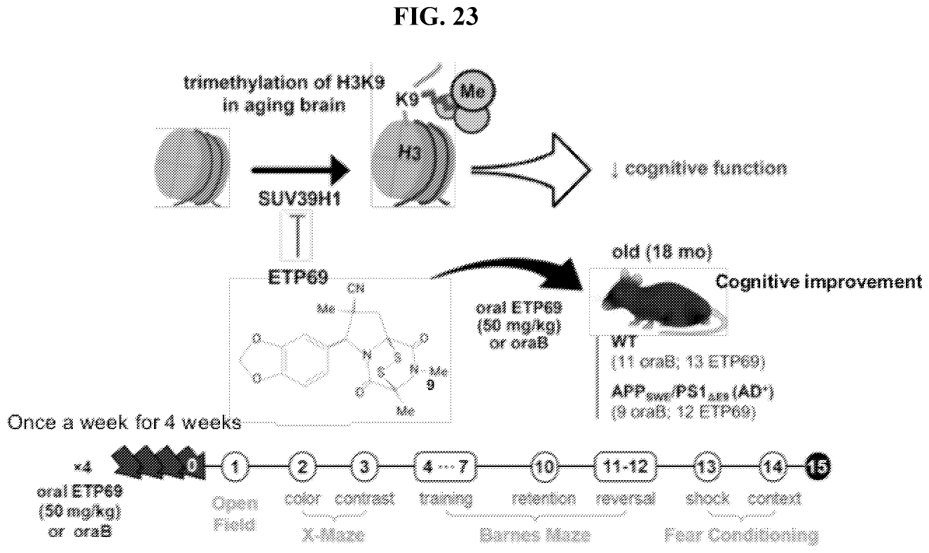

18 month old wildtype and AD+ mice were administered an oral dose of 50 mg/kg ETP69 or oraB once a day for 4 weeks. The experimental protocol is depicted in FIG. 23.

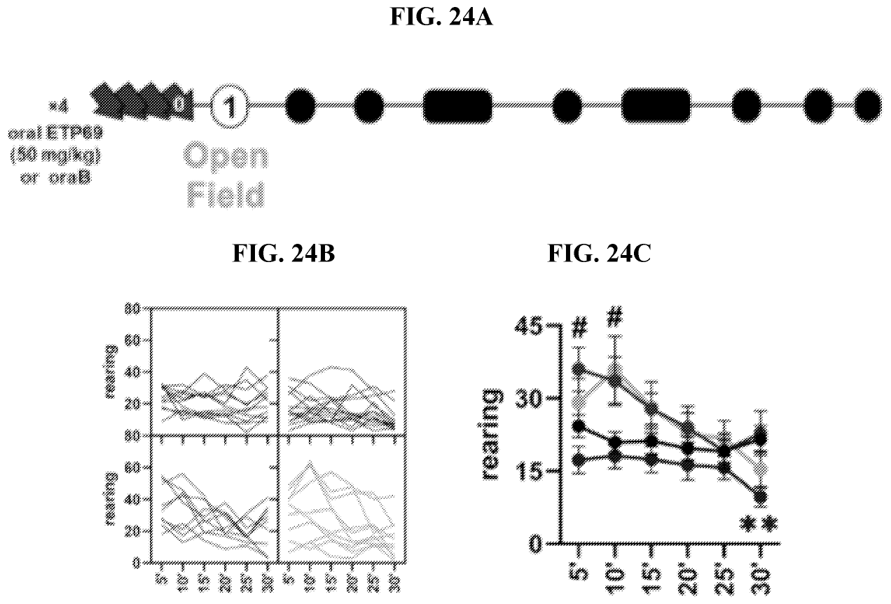

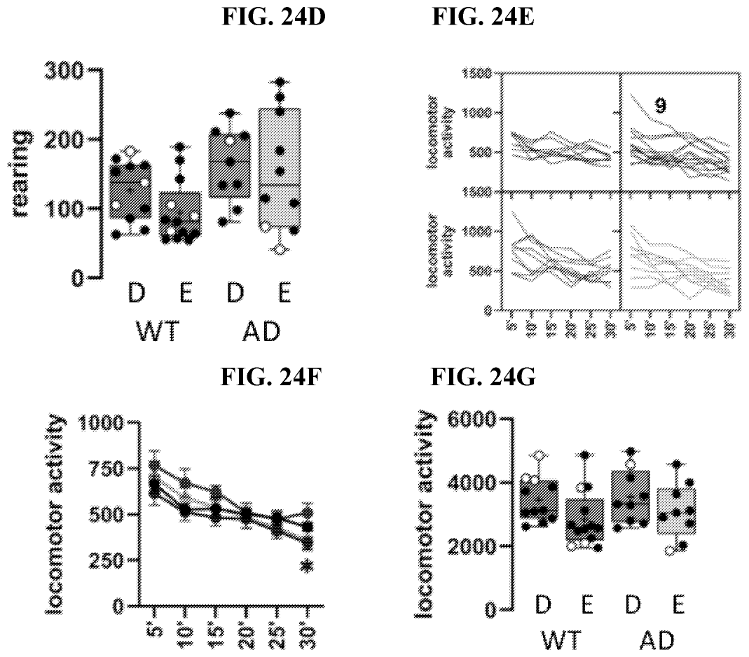

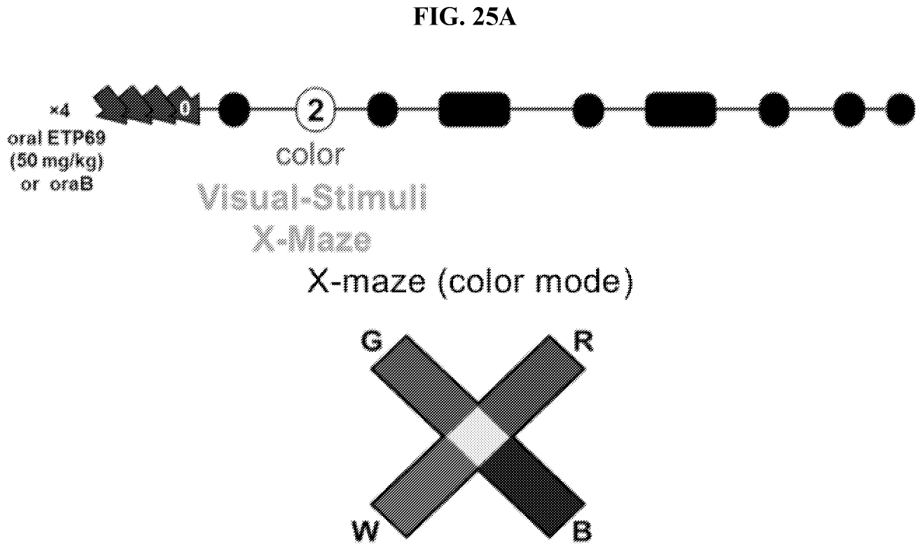

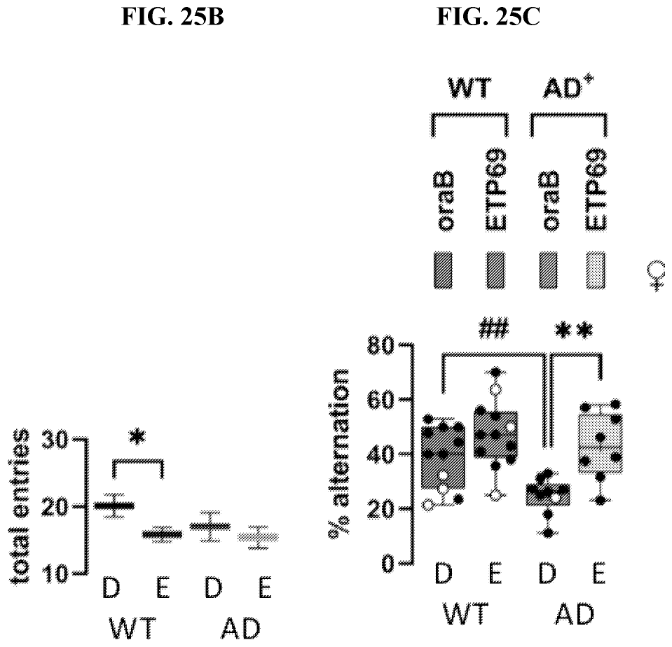

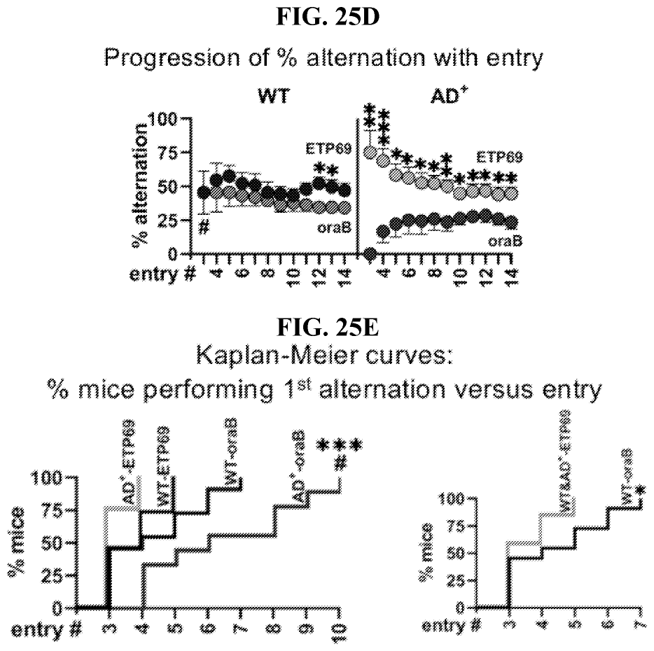

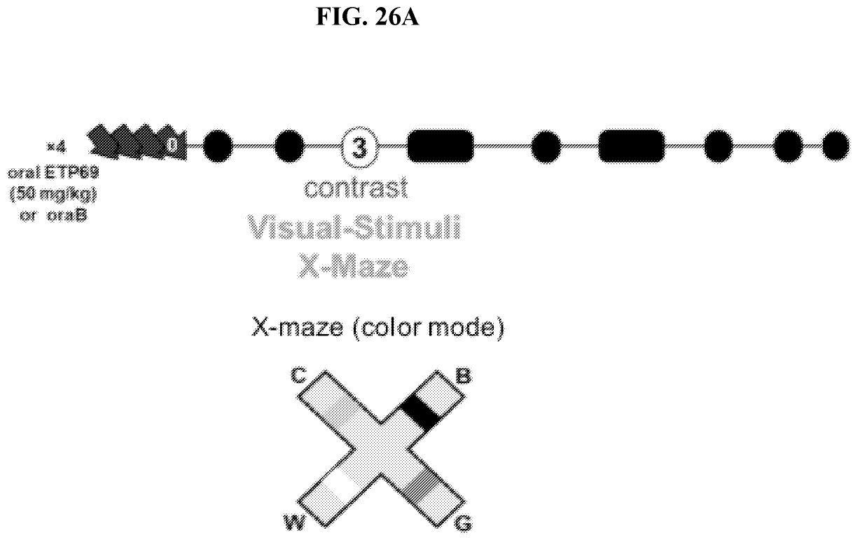

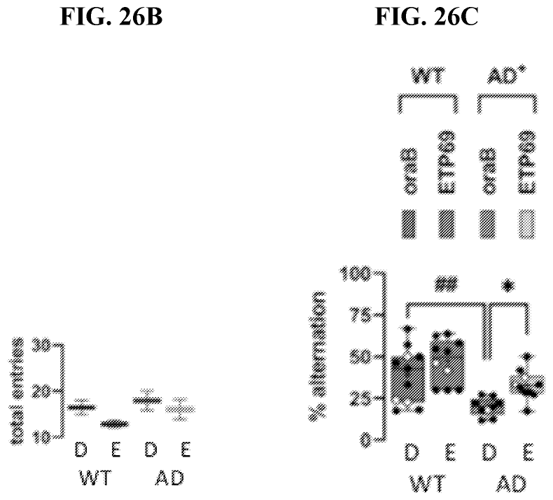

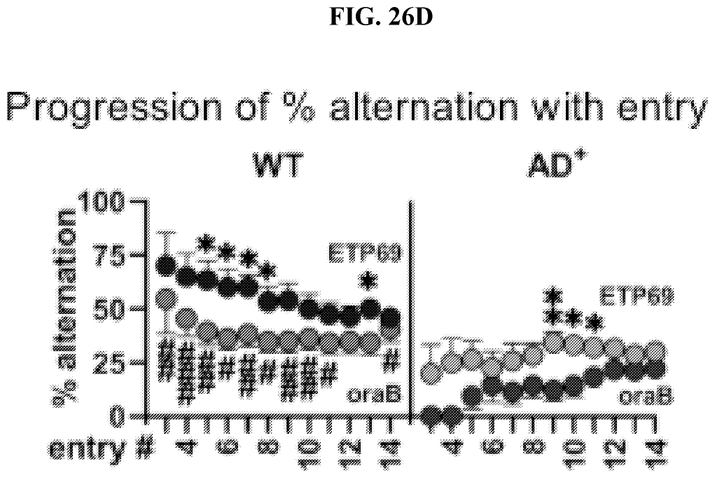

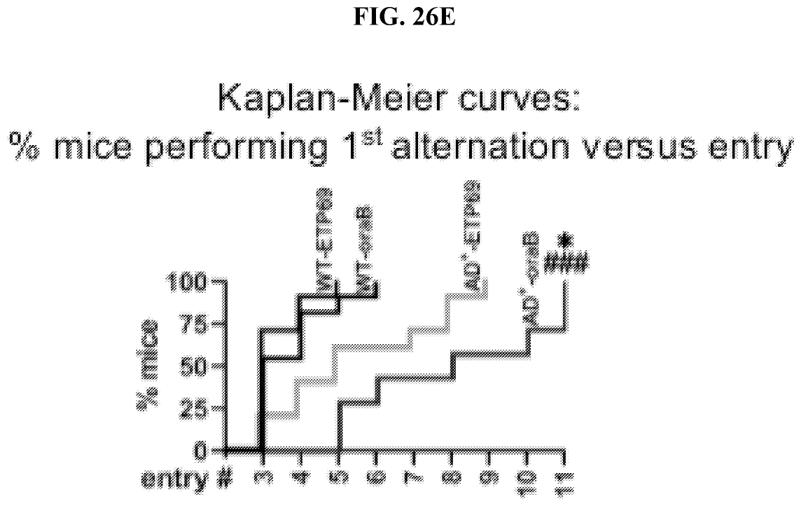

On day 1, the mice were tested in the open field test as depicted in FIG. 24A. Rearing and total locomotor activity were measured in mice administered oraB and ETP69 (FIG. 24B-24G). On day 2, mice were tested in the color visual-stimuli X-maze. The results show the beneficial effects of ETP69-oral formulation in reversing cognitive dysfunction in old mice (FIG. 25A-25E). On day 3, mice were tested in the contrast visual-stimuli X-maze. Results are depicted in FIG. 26A-26E.

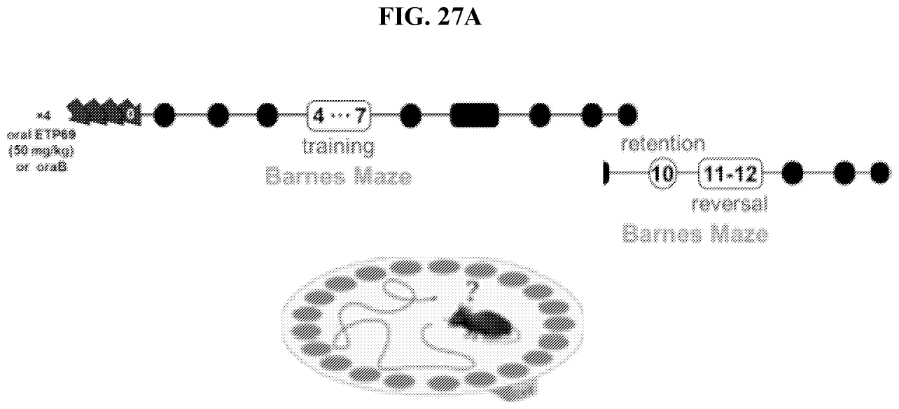

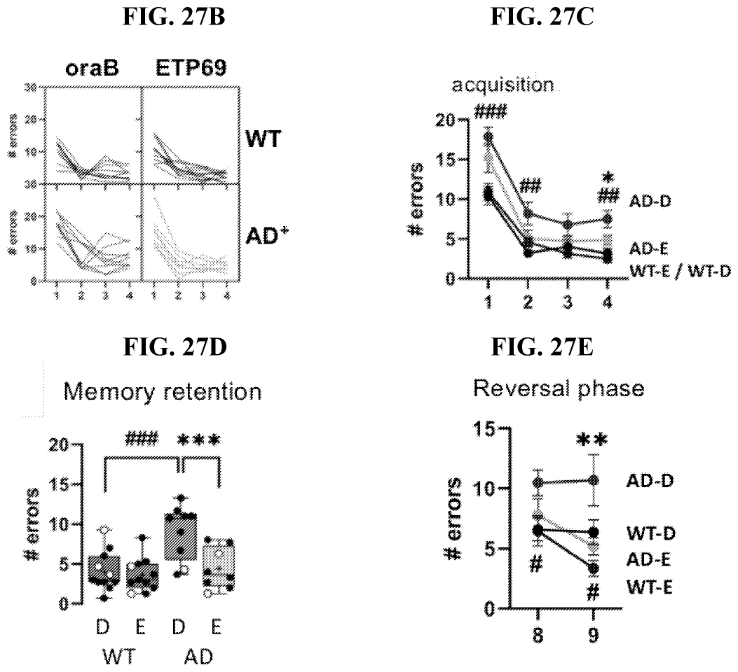

On days 4-7 mice were tested on the training phase of the Barnes Maze (FIG. 27A). On day 10, mice were tested on the retention phase. On days 11-12, mice were tested on the reversal phase. Results are depicted in FIG. 27B-27E. A significant reversal of cognitive deficits following oral ETP69 was seen in old AD-model mice.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of treating, preventing or delaying onset of an Alzheimer's disease in a subject in need thereof, the method comprising administering to the subject a composition comprising ETP69 wherein the subject has (i) at least one mutation associated with familial Alzheimer's disease; or (ii) at least one genetic risk factor associated with sporadic Alzheimer's disease.

2. The method of claim 1, wherein the familial Alzheimer's disease comprises Alzheimer's disease type 1.

3. The method of claim 1, wherein the familial Alzheimer's disease comprises Alzheimer's disease type 3.

4. The method of claim 1, wherein the familial Alzheimer's disease comprises Alzheimer's disease type 4.

5. The method of claim 1, wherein the at least one mutation associated with familial Alzheimer's disease comprises a mutation in an amyloid precursor protein (APP) gene, a presenilin-1 (PSEN1) gene, or a presenilin-2 (PSEN2) gene.

6. The method of claim 5, wherein the mutation in the APP gene codes for a mutation in the amyloid precursor protein amino acid sequence that is expressed from the APP gene.

7. The method of claim 6, wherein the mutation in the amyloid precursor protein amino acid sequence comprises a mutation at valine 717 according to SEQ ID NO: 1.

8. The method of claim 7, wherein the valine 717 of the amyloid precursor protein is mutated to isoleucine (V717I), phenylalanine (V717F), glycine (V717G), or leucine (V717L).

9. The method of claim 6, wherein the mutation in the amyloid precursor protein amino acid sequence comprises mutations at positions lysine 670 and methionine 671 according to SEQ ID NO: 1.

10. The method of claim 9, wherein the lysine 670 and methionine 671 are mutated to asparagine and lysine, respectively (KM670/671NL).

11. The method of claim 6, wherein the mutation in the amyloid precursor protein amino acid sequence comprises a mutation at glutamate 693 according to SEQ ID NO: 1.

12. The method of claim 6, wherein the mutation in the APP gene codes for a deletion at position glutamate 693 of the amyloid precursor protein amino acid.

13. The method of claim 11, wherein the glutamate 693 of the of the amyloid precursor protein is mutated to glutamine (E693Q), glycine (E693G), or lysine (E693K).

14. The method of claim 6, wherein the mutation in the amyloid precursor protein amino acid sequence comprises a mutation at threonine 714 according to SEQ ID NO: 1.

15. The method of claim 14, wherein the threonine 714 of the of the amyloid precursor protein is mutated to isoleucine (T714I) or alanine (T714A).

16. The method of claim 6, wherein the mutation in the amyloid precursor protein amino acid sequence comprises a mutation at valine 715 according to SEQ ID NO: 1.

17. The method of claim 16, wherein the valine 715 of the of the amyloid precursor protein is mutated to methionine (V715M) or alanine (V715A).

18. The method of claim 6, wherein the mutation in the amyloid precursor protein amino acid sequence comprises a mutation at isoleucine 716 according to SEQ ID NO: 1.

19. The method of claim 18, wherein the isoleucine 716 of the of the amyloid precursor protein is mutated to valine (I716V) or phenylalanine (I716F).

20. The method of claim 5, wherein the mutation in the PSEN-1 gene codes for a mutation in the presenilin-1 amino acid sequence that is expressed from the PSEN-1 gene.

21. The method of claim 20, wherein the mutation in the presenilin-1 amino acid sequence comprises a mutation at methionine 146, leucine 166, isoleucine 213, arginine 278, or alanine 246 according to SEQ ID NO: 2.

22. The method of claim 21, wherein the methionine 146 is mutated to leucine (M146L), the leucine 166 is mutated to proline (L166P), the isoleucine 213 is mutated to threonine (I213T), the arginine 278 is mutated to isoleucine (R278I), and the alanine 246 is mutated to glutamate (A246E).

23. The method of claim 2, wherein the mutation in the PSEN-2 gene codes for a mutation in the presenilin-2 amino acid sequence that is expressed from the PSEN-2 gene.

24. The method of claim 23, wherein the mutation in the presenilin-2 amino acid sequence comprises a mutation at asparagine 141 or methionine 239 according to SEQ ID NO: 3.

25. The method of claim 24, wherein the asparagine 141 is mutated to isoleucine and the methionine 239 is mutated to valine.

26. The method of claim 1, wherein the genetic risk factor associated with sporadic Alzheimer's disease comprises the subject being a carrier of an apolipoprotein (APOE) e4 allele.

27. The method of claim 1, wherein the genetic risk factor associated with sporadic Alzheimer's disease comprises a mutation in a gene.

28. The method of claim 27, wherein the gene comprises ABCA7, AKAP9, BIN1, CASS4, CD2AP, CD33, CLU, EPHA1, FERMT2, HLA-DRB5/DRB1, INPP5D, MEF2C, MS4A6A/MS4A4E, PICALM, PLD3, ACE, PTK2B, —SORL1, TREM2, or UNC5C.

29. The method of claim 1, wherein the subject is asymptomatic of Alzheimer's disease.

30. The method of any one of claim 1, wherein the subject is less than about 25, about 30, about 35, about 40, about 45, about 50, about, about 55, about 60, about 65, or about 70 years of age.

31. The method of claim 1, wherein the delaying onset of Alzheimer's disease comprises a delay in onset of at least one symptom of Alzheimer's disease.

32. The method of claim 31, wherein the delay in onset of at least on symptom is at least about 6 months, about 12 months, about 18 months, about 2 years, about 3 years, about 5 years, about 10 years, about 15 years, or about 20 years.

33. The method of claim 31, wherein the symptom comprises memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes.

34. The method of claim 1, wherein the method improves memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes in the subject.

35. The method of claim 34, wherein the treatment results in improvement in a mental status test and/or a neuroimaging test.

36. The method of claim 35, wherein the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test.

37. The method of claim 36, wherein the improvement comprises an improved score relative to a score obtained prior to administration of the composition.

38. The method of claim 35, wherein the neuroimaging test is a magnetic resonance imaging (MRI) or computed tomography (CT).

39. The method of claim 38, wherein the improvement comprises reduced beta-amyloid deposits as compared to an amount of beta-amyloid deposits measured prior to administration of the composition.

40. A method of improving cognition in a subject at risk of developing Alzheimer's disease, the method comprising administering to the subject a composition comprising ETP69 wherein the subject has (i) at least one mutation associated with familial Alzheimer's disease; or (ii) at least one genetic risk factor associated with sporadic Alzheimer's disease.

41. The method of claim 40, wherein the familial Alzheimer's disease comprises Alzheimer's disease type 1.

42. The method of claim 40, wherein the familial Alzheimer's disease comprises Alzheimer's disease type 3.

43. The method of claim 40, wherein the familial Alzheimer's disease comprises Alzheimer's disease type 4.

44. The method of claim 40, wherein the at least one mutation associated with familial Alzheimer's disease comprises a mutation in an amyloid precursor protein (APP) gene, a presenilin-1 (PSEN1) gene, or a presenilin-2 (PSEN2) gene.

45. The method of claim 44, wherein the mutation in the APP gene codes for a mutation in the amyloid precursor protein amino acid sequence that is expressed from the APP gene.

46. The method of claim 45, wherein the mutation in the amyloid precursor protein amino acid sequence comprises a mutation at valine 717 according to SEQ ID NO: 1.

47. The method of claim 46, wherein the valine 717 of the amyloid precursor protein is mutated to isoleucine (V717I), phenylalanine (V717F), glycine (V717G), or leucine (V717L).

48. The method of claim 45, wherein the mutation in the amyloid precursor protein amino acid sequence comprises mutations at positions lysine 670 and methionine 671 according to SEQ ID NO: 1.

49. The method of claim 45, wherein the lysine 670 and methionine 671 are mutated to asparagine and lysine, respectively (KM670/671NL).

50. The method of claim 45, wherein the mutation in the amyloid precursor protein amino acid sequence comprises a mutation at glutamate 693 according to SEQ ID NO: 1.

51. The method of claim 45, wherein the mutation in the APP gene codes for a deletion at position glutamate 693 of the amyloid precursor protein amino acid.

52. The method of claim 51, wherein the glutamate 693 of the of the amyloid precursor protein is mutated to glutamine (E693Q), glycine (E693G), or lysine (E693K).

53. The method of claim 45, wherein the mutation in the amyloid precursor protein amino acid sequence comprises a mutation at threonine 714 according to SEQ ID NO: 1.

54. The method of claim 53, wherein the threonine 714 of the of the amyloid precursor protein is mutated to isoleucine (T714I) or alanine (T714A).

55. The method of claim 45, wherein the mutation in the amyloid precursor protein amino acid sequence comprises a mutation at valine 715 according to SEQ ID NO: 1.

56. The method of claim 55, wherein the valine 715 of the of the amyloid precursor protein is mutated to methionine (V715M) or alanine (V715A).

57. The method of claim 45, wherein the mutation in the amyloid precursor protein amino acid sequence comprises a mutation at isoleucine 716 according to SEQ ID NO: 1.

58. The method of claim 57, wherein the isoleucine 716 of the of the amyloid precursor protein is mutated to valine (I716V) or phenylalanine (I716F).

59. The method of claim 44, wherein the mutation in the PSEN-1 gene codes for a mutation in the presenilin-1 amino acid sequence that is expressed from the PSEN-1 gene.

60. The method of claim 59, wherein the mutation in the presenilin-1 amino acid sequence comprises a mutation at methionine 146, leucine 166, isoleucine 213, arginine 278, or alanine 246 according to SEQ ID NO: 2.

61. The method of claim 60, wherein the methionine 146 is mutated to leucine (M146L), the leucine 166 is mutated to proline (L166P), the isoleucine 213 is mutated to threonine (I213T), the arginine 278 is mutated to isoleucine (R278I), and the alanine 246 is mutated to glutamate (A246E).

62. The method of claim 44, wherein the mutation in the PSEN-2 gene codes for a mutation in the presenilin-2 amino acid sequence that is expressed from the PSEN-2 gene.

63. The method of claim 62, wherein the mutation in the presenilin-2 amino acid sequence comprises a mutation at asparagine 141 or methionine 239 according to SEQ ID NO: 3.

64. The method of claim 63, wherein the asparagine 141 is mutated to isoleucine and the methionine 239 is mutated to valine.

65. The method of claim 40, wherein the genetic risk factor associated with sporadic Alzheimer's disease comprises the subject being a carrier of apolipoprotein (APOE) e4 allele.

66. The method of claim 40, wherein the genetic risk factor associated with sporadic Alzheimer's disease comprises a mutation in a gene.

67. The method of claim 66, wherein the gene comprises ABCA7, AKAP9, BIN1, CASS4, CD2AP, CD33, CLU, EPHA1, FERMT2, HLA-DRB5/DRB1, INPP5D, MEF2C, MS4A6A/MS4A4E, PICALM, PLD3, ACE, PTK2B, —SORL1, TREM2, or UNC5C.

68. The method of claim 40, wherein the subject is asymptomatic of Alzheimer's disease.

69. The method of claim 40, wherein the subject is less than about 25, about 30, about 35, about 40, about 45, about 50, about, about 55, about 60, about 65, or about 70 years of age.

70. The method of claim 40, wherein the method improves memory loss, difficulty concentrating, difficulty completing familiar tasks, confusion with time or place, difficulty understanding visual images and spatial relationships, language difficulties, misplacing items, decreased or poor judgement, social withdrawal, and/or mood or personality changes in the subject.

71. The method of claim 70, wherein the treatment results in improvement in a mental status test and/or a neuroimaging test.

72. The method of claim 71, wherein the mental status test is a Mini-Mental State Exam (MMSE), a Mini-Cog test, a Cantab Mobile test, a Cognigram test, a Cognivue test, or a Cognision and Automated Neuropsychological Assessment Metrics (ANAM) test.

73. The method of claim 72, wherein the improvement comprises an improved score relative to a score obtained prior to administration of the composition.

74. The method of claim 71, wherein the neuroimaging test is a magnetic resonance imaging (MRI) or computed tomography (CT).

75. The method of claim 74, wherein the improvement comprises reduced beta-amyloid deposits as compared to an amount of beta-amyloid deposits measured prior to administration of the composition.

76. The method of claim 1 or 40, wherein the administration results in increased synaptic integrity in the subject as compared to a synaptic integrity measured prior to administration of the composition.

77. The method of claim 1 or 40, wherein the administration results in increased dendritic spine density as compared to a dendritic spine density measured prior to administration of the composition.


SEQUENCE LISTING (3 sequences)

SEQ ID NO: 1 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 467 aa, Homo sapiens
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 3 - protein, 448 aa, Homo sapiens
MLTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYTPFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYI